(19) United States
(12) Patent Application Publication
(10) Pub. No.: US 2022/0154156 A1
(43) Pub. Date: May 19, 2022

(54) PROTEINS AND METHODS FOR DISRUPTING BACTERIAL COMMUNICATION

(71) Applicant: REGENTS OF THE UNIVERSITY OF MINNESOTA, Minneapolis, MN (US)

(72) Inventors: Celine Bergonzi, Le Pecq (FR); Mikael Elias, Centerville, MN (US); Randall Hicks, Duluth, MN (US); Michael Sadowsky, Roseville, MN (US); Michael Schwab, Minneapolis, MN (US)

(21) Appl. No.: 17/437,710

(22) PCT Filed: Mar. 11, 2020
(86) PCT No.: PCT/US20/22031

§ 371 Date: Sep. 9, 2021

Related U.S. Application Data
(60) Provisional application No. 62/816,403, filed on Mar. 11, 2019.
(60) Provisional application No. 62/930,796, filed on Nov. 5, 2019.

(51) Int. Cl. C12N 9/18; A01N 63/50; A01P 1/00

(52) U.S. Cl. CPC C12N 9/18; C12Y 301/01081; A61K 38/00; A01P 1/00; A01N 63/50

(57) ABSTRACT

Provided herein are lactonases. In one embodiment, the lactonases are metallo-β-lactamase-like lactonase (MLL) enzymes having altered characteristics such as altered catalytic activity and/or altered substrate specificity. Also provided are genetically modified microbes able to express a MLL enzyme, compositions that include a MLL enzyme, and methods of using a MLL enzyme.

### CROSS-REFERENCE TO RELATED APPLICATIONS

This application claims the benefit of U.S. Provisional Application Ser. No. 62/816,403, filed Mar. 11, 2019, and U.S. Provisional Application Ser. No. 62/930,796, filed Nov. 5, 2019, each of which are incorporated by reference herein in its entirety.

### GOVERNMENT FUNDING

This invention was made with government support under NA180AR4170101 awarded by the National Oceanic and Atmospheric Administration. The government has certain rights in the invention.

### SEQUENCE LISTING

This application contains a Sequence Listing electronically submitted via EFS-Web to the United States Patent and Trademark Office as an ASCII text file entitled “Seq_Listing_0635WO01_ST25.txt” having a size of 30 kilobytes and created on Mar. 11, 2020. The information contained in the Sequence Listing is incorporated by reference herein.

### BACKGROUND

Bacteria used to be considered solely as individual organisms, whose survival often requires that they outcompete other microorganisms. Bacteria are now known to communicate with each other using a quorum sensing (QS) system. Bacteria use QS to regulate their gene expression, and thereby coordinate actions in a cell density-dependent manner. Bacteria constantly produce small signaling molecules, whose concentration increase proportionally with cell density. When a specific cell density is reached—a “quorum”—a certain concentration of the signaling molecule is reached and will lead to a signal transduction cascade resulting in population-wide changes in gene expression, including the regulation of many traits including virulence and formation of biofilms.

Biofilms are slimy layers of a hydrated matrix of polysaccharides, proteins and nucleic acids produced by bacteria and can attach to surfaces. Biofilms and the result of biofilms, including biofouling and biocorrosion, represent major economic burdens. The use of disinfectants and antibiotics has only had limited success in addressing the problems posed by biofilms and select for resistant strains that represent a threat for human health.

### SUMMARY OF THE APPLICATION

Disruption of bacterial quorum sensing communication has been shown to drastically reduce bacterial biofilms and virulence. Enzymes (proteins), often referred to a lactonases, that degrade the small signaling molecules responsible for bacterial quorum sensing exist but have been difficult to use to address virulence and biofilm formation. The inventors have identified substitutions that can be made to lactonases that result in proteins having properties useful for degrading the small signaling molecules responsible for bacterial quorum sensing.

Provided herein are metallo-β-lactamase-like lactonase (MLL) enzymes. In one embodiment, a MLL protein includes at least one amino acid substitution mutation. The one or more amino acid substitution mutations can be selected from a position functionally equivalent to M21, W25, Q41, F47, S66, S81, T82, M85, F86, T91, R111, L120, F141, A144, C147, E154, A156, G155, V175, H178, 1182, L183, Y222, I237, M244, or N245 in a reference amino acid sequence SEQ ID NO:1. In one embodiment, a MLL protein includes an amino acid sequence that is at least 80% identical to a reference amino acid sequence SEQ ID NO:1, has a lactonase activity, and includes at least one amino acid substitution mutation. The one or more amino acid substitution mutations can be selected from a position functionally equivalent to M21, W25, Q41, F47, S66, S81, T82, M85, F86, T91, R111, L120, F141, A144, C147, E154, A156, G155, V175, H178, 1182, L183, Y222, I237, M244, or N245 in the reference amino acid sequence. The protein can be a fusion protein. This fusion can be between a MLL protein and an affinity purification moiety.

Also provided by the present disclosure are polynucleotides. In one embodiment, a polynucleotide includes (a) a nucleotide sequence encoding a MLL protein described herein, or (b) the full complement of the nucleotide sequence of (a). The polynucleotide can be operably linked to at least one regulatory sequence, and/or can include heterologous nucleotides. The polynucleotide can be present as part of a vector.

The present disclosure provides genetically modified microbes. In one embodiment, a genetically modified microbe includes exogenous polynucleotide where the exogenous polynucleotide encodes a MLL protein described herein, or is the full complement of the polynucleotide encoding a MLL protein.

Further provided by the present disclosure are compositions. In one embodiment, a composition includes a MLL protein described herein. A composition can include a pharmaceutically acceptable carrier, and optionally be formulated for parenteral administration or topical administration to an animal. In one embodiment, a composition is formulated for foliar administration to a plant. In one embodiment, a composition is formulated for use as a coating, a cleaning solution, a feed supplement, or a dietary supplement. In one embodiment, a composition includes a genetically modified microbe described herein. In one embodiment, a composition includes a polynucleotide described herein.

The present disclosure provides articles. In one embodiment, an article includes a composition described herein. The composition can be present on a surface of an article, incorporated into a surface of an article, or a combination thereof.

Also provided by the present disclosure are methods. In one embodiment, a method is for treating an animal infection and includes administering to an animal having or at risk of having an infection an effective amount of a composition described herein. In one embodiment, a method is for treating a sign of a condition and includes administering to an animal having or at risk of having a condition an effective amount of a composition comprising the composition described herein. In one embodiment the animal is a human, and in one embodiment the infection or condition is caused by a gram-negative bacterium or a gram-positive bacterium.

In one embodiment, a method is for treating a plant infection and includes administering to a plant having or at risk of having a bacterial infection an effective amount of a composition described herein. The plant can be a monocot or a dicot, and in one embodiment the infection is caused by a gram-negative bacterium or a gram-positive bacterium. In one embodiment, the administering includes foliar administration.

In one embodiment, a method is for treating a biofilm and includes treating a biofilm present on a surface with an effective amount of one or more MLL proteins described herein. In one embodiment the surface can be one that is at risk of biofilm formation. The surface can include plastic, metal, glass, or a combination thereof. The surface can be impregnated with the protein, coated with the protein, or a combination thereof. In one embodiment, the surface can be part of a medical device such as an endoscope.

In one embodiment, a method is for changing the population of a biofilm and includes treating a biofilm with an effective amount of one or more MLL proteins described herein. In one embodiment, the population is present in a microbiome of, for instance, a human.

In one embodiment, a method is for reducing rot and includes contacting a fruit, fresh produce, fish, meat, or dairy with an effective amount of one or more MLL proteins.

As used herein, the term “protein” refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, tetramers). Thus, the terms peptide, oligopeptide, and protein are all included within the definition of protein and these terms are used interchangeably.

As used herein, a protein may be “structurally similar” to a reference protein if the amino acid sequence of the protein possesses a specified amount of structural similarity and/or structural identity compared to the reference protein. Thus, a protein may have structural similarity to a reference protein if, compared to the reference protein, it possesses a sufficient level of amino acid structural identity, amino acid structural similarity, or a combination thereof.

As used herein, the term “polynucleotide” refers to a polymeric form of nucleotides of any length, either ribonucleotides, deoxynucleotides, or a combination thereof, and includes both single-stranded molecules and double-stranded duplexes. A polynucleotide can be obtained directly from a natural source, or can be prepared with the aid of recombinant, enzymatic, or chemical techniques.

As used herein, the term “exogenous” refers to a polynucleotide or protein that is not normally or naturally found in a specific cell.

An “isolated” polynucleotide or protein is one that has been removed from its natural environment. Polynucleotides and proteins that are produced by recombinant, enzymatic, or chemical techniques are considered to be isolated and purified by definition, since they were never present in a natural environment.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Conditions that are “suitable” for an event to occur are conditions that do not prevent such events from occurring. Thus, these conditions permit, enhance, facilitate, and/or are conducive to the event.

As used herein, “providing” in the context of a composition, an article, a polynucleotide, an article or a protein means making the composition, article, polynucleotide, article or protein, purchasing the composition, article, polynucleotide, article or protein, or otherwise obtaining the composition, article, polynucleotide, article or protein.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1 shows a multiple protein alignment of SEQ ID NOs:1-4. Gdl from Parageobacillus caldoxylosilyticus, SEQ ID NO:1; AaL from Alicyclobacter acidoterrestris, SEQ ID NO:2; AiiA from Bacillus thuringiensis, SEQ ID NO:3; and AiiB from Agrobacterium tumefaciens, SEQ ID NO:4. The five histidines and two aspartic acids present in GcL active site and implicated in metals coordination are underlined in bold. The amino acids of the Metallo-β-lactamases-like-lactonases conserve sequence, HXHXDH, are highlighted with “\hat{ }”. The amino acid of the active site cavity are highlighted with “#”. The amino acids ATIHNPNAQ (residues 33-41 of GcL), ATIANPNAP (residues 33-41 of AaL), and VTPQKPTVS (residues 27-35 of AiiB) represent the insertion allowing the dimerization present in GcL, AaL and AiiB but not in AiiA. An “*” (asterisk) indicates positions which have a single, fully conserved residue. A “:” (colon) indicates conservation between groups of strongly similar properties roughly equivalent to scoring >0.5 in the Gonnet PAM 250 matrix. A “.” (period) indicates conservation between groups of weakly similar properties as below—roughly equivalent to scoring=<0.5 and >0 in the Gonnet PAM 250 matrix.

FIG. 2 shows a multiple protein alignment of SEQ ID NOs:5-7. SsoPox from Sulfolobus solfataricus, SEQ ID NO:5; SisLac from Sulfolobus islandicus, SEQ ID NO:6; and VmoLac from Vulcanisaeta moutnovskia, SEQ ID NO:7. Metal coordinating residues are underlined in bold. An “*” (asterisk) indicates positions which have a single, fully conserved residue. A “:” (colon) indicates conservation between groups of strongly similar properties roughly equivalent to scoring >0.5 in the Gonnet PAM 250 matrix. A “.” (period) indicates conservation between groups of weakly similar properties as below—roughly equivalent to scoring=<0.5 and >0 in the Gonnet PAM 250 matrix.

FIG. 3A-3G show substrates tested in this study. (A.) Acyl-Homoserine Lactones, (B.) 3-oxo-Acyl-Homoserine Lactones, (C.) γ-lactones, (D.) δ-lactones, (E.) ε-caprolactone, (F.) Whiskey-lactone and (G.) Paraoxon-ethyl.

FIG. 4A-4F show structure of GcL bound to HEPES. (A.) GcL homodimer. The monomer A is colored in blue and monomer B in grey. (B.) GcL homodimer (in blue and grey). (C.) GcL structure showing a αβ/βα fold with 6 α-helix in yellow and 10 β-strands in red. The active site is depicted by the presence of the catalytic water (red sphere) and the α-cobalt and the β-iron cations, in pink and orange respectively. (D.) Metals' coordination in GcL. The interactions with the catalytic water molecule the metal cations and active site residues are represented as green sticks. (E.) GcL active site bound to a HEPES molecule (purple sticks) and the electron density is shown as a blue mesh (2Fo-Fc, contoured at 0.85 σ). (F.) Interaction of the HEPES molecule with the bi-metallic center. In these figures, the two metal cations are shown as spheres (α-cobalt in pink; β-iron in orange) and the catalytic water molecule We is shown as a red sphere.

FIG. 5A-5B show active site comparison between GcL, AiiA and AiiB. (A.) The Gdl structure (in grey sticks, pink and orange metal cations, and residues labeled in black) was superposed to AiiA structure (in blue sticks, purple metal cations, and residues labeled in blue). The main differences correspond to a different orientation of Y223, a slight movement of H198 and H265, a different position of the catalytic water and A206 (AiiA)/1237 (GcL). (B.) The Gdl structure (in grey sticks, pink and orange metal cations, and residues labeled in black) was superposed to AiiB structure (in pink sticks, grey metal cations, and residues labeled in pink). The metals coordination is different between the two structures and is visible through different positions of D122 and D220, H120, H198 and H265. The GcL residue I237 is corresponding to a V230 in AiiB.

FIG. 6A-6F show GcL bound to C6 AHL. Metal cations of GcL are shown as pink (α-cobalt) and orange (β-iron) spheres and the catalytic water molecule is shown as a red sphere. Distances are indicated in Ångstroms. (A) Electronic density for C6-AHL complex structure (purple). The 2Fo-2Fc map is contoured at 1.0 σ. The hydrophobic channel residues of the GcL are represented in green stick and GcL hydrophobic patch (W26, F86, I237) interacts with the acyl chain. (B.) Interactions of the C6-AHL (orange sticks) with the active site residue Y223 and with the bimetallic active site. The bridging are represented by black dashes. (C). Superposition of the HEPES-bound structure (grey sticks) and the C6-AHL bound GcL structure (green sticks). C6-AHL molecule is shown as orange sticks. 1237 adopts two different conformations in these structures. (D) Superposition of the C6-AHL bound GcL structure (grey sticks) and the C6-AHL hydrolytic product bound AiiA structure (blue sticks; PDB: 3DHB). C6 AHL and the hydrolytic product are shown as orange and cyan sticks, respectively. Metal cations of GcL (pink) and AaL (grey) and their bridging water molecules (red) are shown as spheres. (E.) Superposition of the C6-AHL bound GcL structure (green sticks) and the C6-AHL bound AaL structure (grey stick). The binding of the C6-AHL substrates (cyan sticks in AaL and orange sticks in GcL) reveals two different orientations of the lactone rings. The carbonyl oxygen of the lactone ring is located at 3.2 Å from Y223 in GcL whereas it is 4.5 Å in AaL. (F.) Binding of C6-AHL in AaL (cyan sticks) and GcL (orange sticks). Distance between metal cations (3.5 Å) in GcL is shorter than in AaL (4.0 Å). This difference in the metal coordination impacts the position of residue D220 (green in GcL and gray in AaL). Distances are indicated for AaL (black) and for GcL (red) as dashes.

FIG. 7A-7C show comparison of the active site access in GcL and AiiA structures. (A). Surface of the GcL monomer (in grey) bound to a C6-AHL (orange stick). The framed image represents a zoom in the active site cavity. (B). Surface of the AiiA monomer (in grey) bound to a C10-AHL product (cyan stick) (PDB: 3DHB). The frames pictures represent a zoom into the different active site access. (C). Superposition of GcL and AiiA bound structures. GcL surface is shown (grey), as well as bound C6-AHL (orange sticks) and the hydrolytic product of C10-AHL (cyan sticks).

FIG. 8A-8B show GcL bound to ε-caprolactone. (A.) Interaction of the lactone ring (purple sticks) with the bi-metallic active site and surrounding residues (green sticks). (B.) Superposition of C6-AHL bound GcL structure (orange sticks) and the ε-caprolactone bound GcL structure (purple sticks). Distances are indicated in Ångstroms. Metal cations are shown as pink (α-cobalt) and orange (β-iron) spheres and the catalytic water molecule is shown as a red sphere.

FIG. 9A-9D Comparison of Gdl structures with other MLL representatives. (A) Superposition of GcL dimer in grey with AiiB dimer (PDB: 2R2D) in pink. (B.) Superposition of GcL (grey) and AaL monomers (green) (C.) Superpositon of GcL monomer (grey) and AiiA monomer in blue (PDB: 2A7M). (D.) Superpositon of GcL monomer in grey with AiiB monomer (PDB: 2R2D) in pink.

FIG. 10 A-10C show anomalous scattering discriminates between metals in GcL active site. (A) Final model and Bijvoet difference fourier map contoured at 5.0 σ of the data collected below the CO—K edge. (B) Final model and Bijvoet difference fourier map contoured at 5.0σ of the collect above the CO—K edge. This clearly demonstrated that the β site (left) is occupied by an iron cation and the β site (right) by a cobalt cation. (C) The scale representing zinc, cobalt and iron levels indicate characteristic absorption Kα and Kβ edges

FIG. 11 shows Hydrophobic pocket of GcL in grey and AaL proteins. We observed than both proteins possess the same residues with form a high hydrophobic pocket in which the acyl chain of the C6 HSL is plated on.

FIG. 12 A-12B show GcL monomers with mutations highlighted in blue (A; GcL 4) and in red (B; GcL 14).

FIG. 13 shows SDS-Page Gel of cultures (whole cells) for Gdl wt, GcL 4 and GcL 14 after overnight induction at 18° C.

FIG. 14 shows melting Temperature of Gdl wt (red), GcL 4 (blue) and GcL 14 (green).

FIG. 15 A-15C Comparison of GcL wt, GcL 4 and GcL 14 structures. (A) Monomers of GcL wt (grey), GcL 4 (blue) and GcL 14 (pink). (B) Active sites of GcL wt (grey), GcL 4 (blue) and GcL 14 (pink). Metal cations and the bridging catalytic water molecule are shown as spheres (C) Representation of the thermal motion B-factor in the structures of GcL wt (left), GcL 4 (middle) and GcL 14 (right). A thick and red ribbon represents structural areas where mobility is high, whereas a thin and blue ribbon highlights areas where mobility is low. Changes in active sites' loop mobility are highlighted by black arrows.

FIG. 16 shows crystal structures of GcL in complex with lactones of different chain length reveal key structural determinants for its activity and substrate specificity. Overlay of unpublished crystal structures of the MBL GcL (green) bound to C4 AHL (grey sticks), C6-AHL (cyan sticks) and 3-oxo C12 AHL (pink sticks) at resolutions between 1.6-1.8 Å. This is the first MBL family structure crystalized with a bound substrate. Highlighted zones indicate the lactone ring binding subsite (yellow area) and the unique hydrophobic patch involved in the AHL acyl chain accommodation. Structure accounts for the lower KM value of GcL than other MBLs such as AiiA (orange area) and the residues interacting with long acyl chains (green area), possibly involved in substrate specificity.

FIG. 17 shows ratios of normalized activity (with wild-type (wt) as reference) of I236X mutants against three different lactones. Green colors indicate activity levels superior to wild-type (wt), whereas red colors indicate activity levels lower than wild-type. Substrate activity ratios (C1/C4; C4/C8; C1/C8) are colored in greed when improved and red when decreased, as compared to wild type (wt).

FIG. 18 shows ratios of normalized activity (with wild-type (wt) as reference) of A156X mutants against three different lactones. Green colors indicate activity levels superior to wild-type (wt), whereas red colors indicate activity levels lower than wild-type. Substrate activity ratios (C1/C4; C4/C8; C1/C8) are colored in greed when improved and red when decreased, as compared to wild type (wt).

FIG. 19 shows residues predicted to change for each reconstructed ancestors (node 55) highlighted as spheres on the Ssopox wt structure.

FIG. 20 shows SDS-Page Gel of E. coli cells lysates (supernatants) for SsoPox wt, SsoPox 6 and SsoPox 19 variants.

FIG. 21 shows quantification of expressed proteins in E. coli cells lysates (supernatants) for SsoPox wt, SsoPox-W263I, SsoPox 6 and SsoPox 19 variants.

FIG. 22 shows paraoxonase activity of SsoPox variants incubated at 80° C. as a function of time.

FIG. 23 shows comparison of the catalytic efficiencies (kcat/KM (s−1M−1)) of SsoPox variants for various substrates.

FIG. 24 shows schematic of the experimental microcosm.

FIG. 25 shows antifouling activity of an acrylic-lactonase coating. Various polycarbonate samples were submerged in Lake Minnetonka (Tonka bay marina) for 1 month. Coatings were prepared with 200 μg/mL of control ingredients (BSA or copper oxide (CuO)) and of lactonases (SsoPox and GcL). Large zebra mussels are circled.

FIG. 26 shows number and percent coverage of corrosion tubercles, and surface roughness measurement on steel coupons with different experimental treatments. Mean values are shown (n=3).

FIG. 27A-27F show photographic and SEM images of steel coupons after exposure. A, D: Silica gel only control. B, E: Surfactin silica gel treatment. C, F: Lactonase silica gel treatment. The red box in SEM image F indicates silica gel coating was peeling off during exposure.

FIG. 28 shows heatmap comparison of the abundance and diversity of the top 50 bacteria order level taxonomy across triplicate samples. All sequence data were used to calculate the relative abundance. Diversity is indicated by OTU Counts in each order.

FIG. 29 shows number and percent coverage of corrosion tubercles, and surface roughness measurement on steel coupons with experimental treatments of different enzyme concentrations after exposure to Duluth-Superior Harbor water for 7 weeks. Mean values are shown (n=3).

FIG. 30A-30F show control of plant infections by a single spraying of 1 mL of lactonase solution (10 mg/mL) on plant leaves and crop. (A) and (B) Soft rot potato assay. Potato slices were infected with 5*10 7cells of Pectobacterium carotovorum CIR354 and treated with an inactive enzyme (control (A)) or with the active enzyme (B). The absence of black areas in (B) indicates less maceration. (C) and (D) Barley leaves were infected with 108 cells of Xanthomonas translucens pv. translucens LMG876 and treated with an inactive enzyme (control (C)) or with the active enzyme (D). Absence of leaf spots in (D) indicates protection. (E) and (F) Wheat leaves were infected with 108 cells of Xanthomonas translucens pv. undulosa LMG892 and treated with an inactive enzyme (control (E)) or with the active enzyme (F). Absence of leaf spots in (F) indicates protection.

FIG. 31 show control of plant infections by a single spraying of 1 mL of lactonase solution (10 mg/mL) on corn leaves. The Viking maize seed (40-30UP) were grown in Euro pot (diameter 8 inch) with sterilized soil mixture (50 standard soil/50 Germinating Mix) in a green house under diurnal conditions with 16 hours lights at 22° C. and 8 hours dark at 18° C. Plant leaves were infected with Clabibacter michiganensis subsp. nebraskensis by dipping the clipped leaf into cell suspension (105cell/mL). Shown are duplicates plants for the control treatment (left) and lactonase treatment (right).

FIG. 32 shows QQ lactonase protects plants from bacterial infection. Plants were inoculated twice (day 1 and day 6) with 5×106 cells P. syringae pv. Phaseolicola. Left, leaves were sprayed with enzyme buffer after the fist inoculation, for right, leaves were sprayed with buffer containing the QQ lactonase (0.5 mg/mL). Pictures were taken at day 14.

FIG. 33 shows QQ lactonase protects plants from bacterial infection. Kidney bean plants were inoculated once with 5×106 cells P. syringae pv. Phaseolicola. Leaves were sprayed with enzyme buffer (control), or sprayed with buffer containing the QQ lactonase at varying concentrations. Leaves were harvested 4 days post inoculation.

FIG. 34A-34C show protection of Caenorhabditis elegans from infection by Pseudomonas aeruginosa by lactonases. Slow-killing, gut infection assay performed with P. aeruginosa. Lactonases SsoPox (A) and GcL (B) are sprayed in the petri dish plate, and final concentrations are given. (C) Paralysis assay by P. aeruginosa, and treatment with SsoPox. Used controls are Bovine Serum Albumine (BSA), quorum sensing P. aeruginosa mutant LasR-, and SsoPox 5A8 mutant, an inactive enzyme mutant. OP50 Escherichia coli strain serves as a non-virulent control.

FIG. 35 shows schematic representation of the experimental system. Bacterial communities were cultured in a tank vessel. A peristaltic pump pumps the culture media through a filtration cartridge made of silica beads. The beads entrap E. coli cells that overproduced an engineered quorum quenching lactonase. As the system operates, the N-acyl homoserine lactone molecules (AHLs) produced by cultured bacteria are enzymatically degraded by the filtration cartridge.

FIG. 36A-36B show functionalized silica gel enzymatic activities and durability. (A) Lactone hydrolysis activity of the engineered silica gels containing the lactonase SsoPox W263I using C8-AHL and γ-undecanoic lactone as substrates. Lactonase activity is expressed in enzymatic units defined as μM of substrate hydrolyzed per min per mg of cells. (B) Activity of the enzymatic silica gels over time, using the chromogenic substrate paraoxon as a proxy for enzyme activity.

FIG. 37A-37B show bioreactors' parameters over the time course of the experiment (21 days). Measurements were performed on the three distinct bioreactors equipped with different filtration cartridges: the 2× lactonase cartridge, containing only lactonase beads (blue line), the control cartridge containing only control beads (dark line) and (c) the 1× lactonase cartridge containing a 1:1 ratio of lactonase beads and control beads (green line). (A) Bacterial growth as measurement by the optical density at 600 nm. (B) Biofilm quantification in submerged wells as quantified by Crystal violet binding measured at 550 nm.

FIG. 38 shows presence of lactonase reduces biofilm formation on sample glass slides in the bioreactors. Glass slips submerged in the bioreactors were stained using Sybr DNA stain and visualized using a 60× magnification.

FIG. 39 shows bacterial community changes as a function of lactonase concentration and time. Analysis were performed using 16S v4 rRNA sequencing data. Relative abundance of bacteria at the genus level in the three different bioreactors (2× lactonase; 1× lactonase and control) over time (from day 4 to day 18).

FIG. 40A-40B show bacterial community changes as a function of lactonase concentration and time. Analysis were performed using 16S v4 rRNA sequencing data. Principal Coordinate analysis of microbial communities over time (from day 4 to day 18). Analysis are performed for (A) the 2× lactonase (blue squares) and control communities (grey circles) and (B) the 1× lactonase (green triangles) and control communities (grey circles).

FIG. 41A-41B show suspension bacterial community changes in presence or absence of active lactonase. Analysis were performed using 16S v4 rRNA sequencing data. (A) Relative abundance of bacteria at the genus level for community treated with the inactive enzyme (control) and the active enzyme (SsoPox-W2361) at two different times (days 3 and 7). (B) Principal Coordinate analysis of microbial communities in presence (red) or absence (grey) of active lactonase. Data for day 3 and 7 are shown as squares and circles, respectively.

FIG. 42 shows bioreactor pH values over the time course of the experiment (21 days). Measurements were performed on the three distinct bioreactors equipped with different filtration cartridges: the 2× lactonase cartridge, containing only lactonase beads (blue line), the control cartridge containing only control beads (dark line) and the 1× lactonase cartridge containing a 1:1 ratio of lactonase beads and control beads (green line). pH monitoring over the time-course of the experiment.

FIG. 43 shows biofilm quantification using Crystal Biolet dye. 96-well plates with detachable wells were submerged in the bioreactors and wells were sampled at various times for biofilm quantification using Crystal Violet dye. Staining is visibly reduced in presence of the highest lactonase concentration (2× lactonase), as compared to the lower lactonase concentration (1×) and control.

FIG. 44 shows biofilm dry weight in tubing at the end of the experiment (21 days). Sections of silicone tubing (15.0 cm) were cut and dried for 24 hours, and weighted on a precision balance. Dry weights are shown after subtracting the weight of a biofilm free section of identical tubing.

FIG. 45 Bacterial Community changes as a function of lactonase concentration and time. Analysis were performed using 16S v4 rRNA sequencing data. Principal Coordinate analysis of microbial communities over time (from day 4 to day 18). Analysis are performed for the 2× (blue squares), 1× lactonase (green triangles) and control communities (grey circles).

FIG. 46A-46B show shannon index and number of observed species in the biofilm communities. Analyses were performed using 16S v4 rRNA sequencing data. Blue squares, green diamonds and black triangles are for 2× lactonase, 1× lactonase and control treated bioreactors, respectively. (A) Shannon indexes were calculated for the different microbial communities over time (from day 4 to day 18). (B) Number of different species observed in the different microbial communities over time (from day 4 to day 18).

FIG. 47 show different lactonases induce differential planktonic microbial community biases. Principal component analysis (PCA) based on deep bacterial 16S rDNA sequences (Illumina V4 region). Experimental bioreactors, developed in our lab, run in triplicates over 7 days, were inoculated with a soil bacterial community treated with active lactonases (red is SsoPox W263I, green is GcL) or with a control protein (inactive lactonase: SsoPox 5A8).

### DETAILED DESCRIPTION OF ILLUSTRATIVE EMBODIMENTS

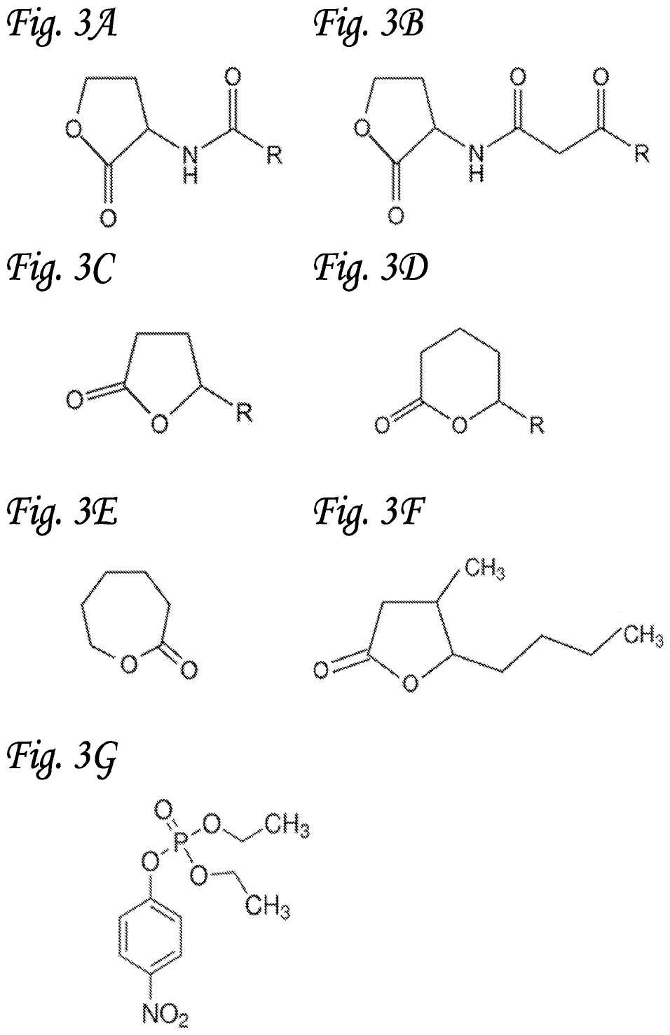

The present disclosure provides isolated proteins having lactonase activity. Two types of proteins having lactonase activity are described herein: MLL lactonases and PLL lactonases. Both MLL lactonases and PLL lactonases catalytically alter the structure of a N-acyl homoserine lactone (AHL). AHL molecules altered by a lactonase described herein include an (S)-α-amino-γ-butyrolactone ring that is linked to an alkyl chain by an amide bond. This and other lactone molecules that can be altered by a lactonase are shown in Table 1 and FIG. 3, where R is an alkyl chain. The alkyl chain can vary in length. In one embodiment, the alkyl chain has at least 4, at least 5, at least 6, at least 7, at least 8, at least 9, at least 10, at least 11, at least 12, at least 13, at least 14, or greater than 14 carbon atoms. In one embodiment, the alkyl chain has no greater than 20, no greater than 19, no greater than 18, no greater than 17, no greater than 16, no greater than 15, no greater than 14, no greater than 13, no greater than 12, no greater than 11, no greater than 10, no greater than 9, no greater than 8, no greater than 7, no greater than 6, or no greater than 5 carbon atoms. The alkyl chain can be linear or branched, and in one embodiment is linear. The alkyl chain can be saturated or unsaturated, and in one embodiment in saturated. Optionally, the alkyl chain can include one or more modifications such as, but not limited to, oxidation at carbon 3 (Lade et al., 2014, Biomed. Res. Int. 2014, 162584) or a p-coumaroyl substituent (Schaefer et al., 2008, Nature, 454, 595-599). In one embodiment, the alteration is the opening of the lactone ring to generate a proton. Specific but non-limiting examples of AHL substrates that can be altered by a lactonase include, but are not limited to, C4-AHL, C6-AHL, C8-AHL, C10-AHL, C12-AHL, 3-oxo-C8-AHL and 3-oxo-C12-AHL. Other AHL compounds that are substrates of a lactonase include, but are not limited to γ-Butyrolactone, γ-Heptalactone, γ-Nonalactone, γ-Decanolactone, δ-Valerolactone, δ-Octanolactone, δ-Nonalactone, δ-Decalactone, ε-Caprolactone, ε-Decalactone, γ-Heptanolide, and Whiskey lactone (see Table 1).

TABLE 1
Example of structures of AHL, γ- and δ- lactones that are substrates of lactonases.

| Compound | Structure |
|---|---|
| C4-AHL | 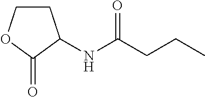 |
| C6-AHL | 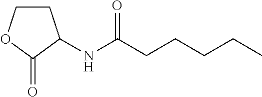 |
| C10-AHL | 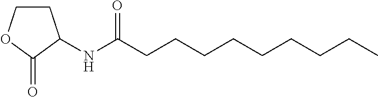 [SMILES: CCCCCCCCCC(=O)NC1CCOC1=O] |
| 3-Oxo-C12-AHL | 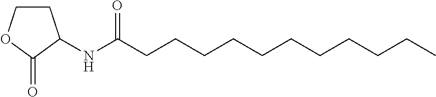 [SMILES: CCCCCCCCCCCC(=O)NC1CCOC1=O] |
| γ-butyrolactane | 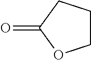 |
| γ-heptanolide | 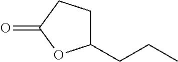 |
| δ-valerolactone | 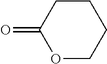 |
| δ-decalactone | 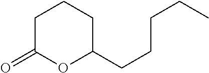 |

### MILL Proteins

In one embodiment, a protein that catalytically alters the structure of an AHL is referred to herein as a MLL lactonase. Whether a protein has MLL lactonase activity can be determined by in vitro assays. In one embodiment, an in vitro assay is carried out by using a pH indicator assay as described in the Examples. Briefly, a lactone hydrolysis assay can be performed in lactonase buffer (Bicine 2.5 mM pH 8.3, NaCl 150 mM, CoCl2 0.2 mM, Cresol purple 0.25 mM and 0.5% DMSO). The cresol purple (pKa 8.3 at 25° C.) is a pH indicator following the lactone ring hydrolysis by media acidification (molar extinction coefficient at F577 nm=2 923 M−1 cm−1). In one embodiment, the substrate is, C6-AHL.

A MLL lactonase described herein is a member of the metallo-β-lactamase-like lactonases (MLL) family (Fetzner, 2015, J. Biotechnol., 201, 2-14). The MLL lactonases exhibit a conserved dinuclear metal binding motif, HXHXDH (SEQ ID NO:15, wherein X is any amino acid), involved in the binding of two metal cations and possess an αβ/βα fold (LaSarre and Federle, 2013, Microbiol. Mol. Biol. Rev. MMBR 77, 73-111). The first discovered member of the MLL family, autoinducer inactivator A (AiiA), was isolated from Bacillus thuringiensis. Its crystal structure has been solved and its catalytic mechanism has been investigated (Liu et al., 2005, Proc. Natl Acad. Sci. USA, 102, 11882-11887). Numerous MLLs have been isolated and characterized, and the structures of AiiA (Liu et al. 2005, Proc. Natl. Acad. Sci. U.S.A 102, 11882-11887; Kim et al. 2005, Proc. Natl. Acad. Sci. U.S.A 102, 17606-17611; Liu et al. 2008, Biochemistry (Mosc.) 47, 7706-7714; and Momb et al. 2008, Biochemistry (Mosc.) 47, 7715-7725), AiiB from Agrobacterium tumefaciens (Liu et al. 2007, Biochemistry (Mosc.) 46, 11789-11799), AidC from Chrysseobacterium sp. Strain StRB126 (Mascarenhas et al. 2015, Biochemistry (Mosc.) 54, 4342-4353) and AaL from Alicyclobacter acidoterrestris (Bergonzi et al., 2017, Acta Crystallogr. Sect. F 73, 476-48; Bergonzi et al., Scientific Reports. 8:11262) have been resolved. The active site of MLLs is composed of a bi-metallic nuclear center bridged by a putative catalytic water molecule that is hypothesized to attack the electrophilic carbon atom of the lactone ring (LaSarre and Federle, 2013, Microbiol. Mol. Biol. Rev. MMBR 77, 73-111). MLLs possess broad substrate preference (Fetzner, 2015, J. Biotechnol. 201:2-14; Bergonzi et al., Scientific Reports. 8:11262).

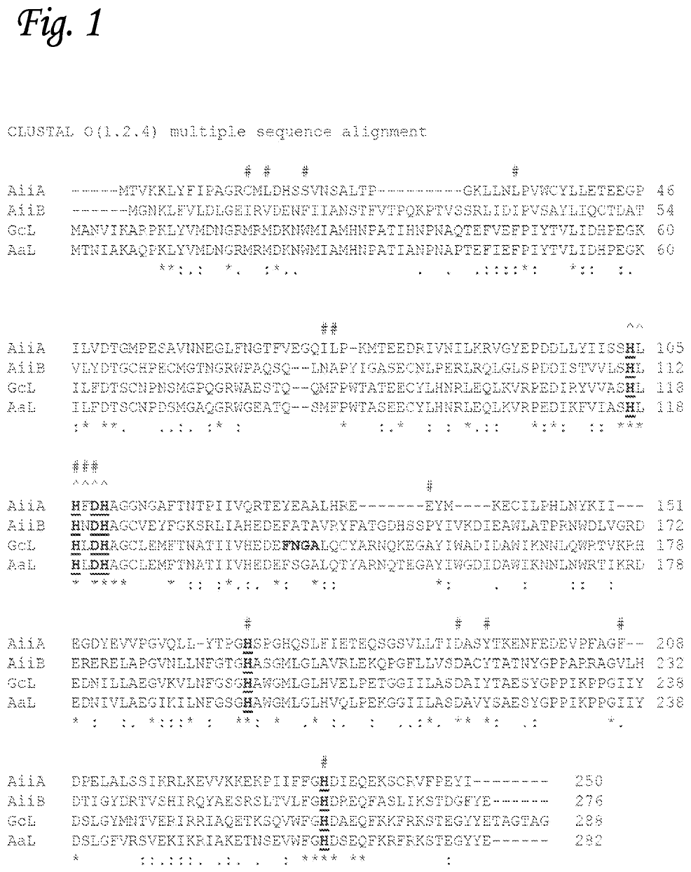

Examples of MLL lactonase proteins are depicted at SEQ ID NOs:1, 2, 3, and 4. FIG. 1 shows an alignment of SEQ ID NOs:1-4 and conserved features. The conserved dinuclear metal binding motif is present at amino acids 117-122 of SEQ ID NO:1. The five histidines and two aspartic acids present in the SEQ ID NO:1 active site and are implicated in metals coordination are shown in bold underline. The amino acids of the active site cavity are present a residues 19, 21, 25, 47, 85, 86, 118, 120, 156, 197, 219, 222, and 236 of SEQ ID NO:1. The main α-helices correspond to residues 80-85, 96-103, 138-150, 159-168, and 240-257 of SEQ ID NO:1) and 0-sheets correspond to 10-18, 47-55, 133-137, 190-194, and 201-207 of SEQ ID NO:1. The amino acids corresponding to residues 33-41 of SEQ ID NO:1 represent the insertion allowing the dimerization present in the GcL, AaL and AiiB but not the AiiA protein.

A MLL lactonase protein described herein includes one or more amino acid substitutions (also referred to as mutations) in comparison to a reference MLL lactonase protein. The amino acid substitutions are described herein. Other examples of MLL lactonase proteins of the present disclosure include those having structural similarity with the amino acid sequence of SEQ ID NO:1, 2, 3, or 4. A lactonase protein having structural similarity with the amino acid sequence of SEQ ID NO: 1, 2, 3, or 4 has MLL lactonase activity. A MLL lactonase protein can be isolated from a microbe or can be produced using recombinant techniques, or chemically or enzymatically synthesized using routine methods. Methods for determining whether a protein has structural similarity with the amino acid sequence of SEQ ID NO:1, 2, 3, or 4 are described herein.

The amino acid sequence of a MLL lactonase protein having structural similarity to SEQ ID NO:1, 2, 3, or 4 can include conservative substitutions of amino acids present in SEQ ID NO:1, 2, 3, or 4. A conservative substitution is typically the substitution of one amino acid for another that is a member of the same class. For example, it is well known in the art of protein biochemistry that an amino acid belonging to a grouping of amino acids having a particular size or characteristic (such as charge, hydrophobicity, and/or hydrophilicity) may generally be substituted for another amino acid without substantially altering the secondary and/or tertiary structure of a polypeptide. For the purposes of this invention, conservative amino acid substitutions are defined to result from exchange of amino acids residues from within one of the following classes of residues: Class I: Gly, Ala, Val, Leu, and Ile (representing aliphatic side chains); Class II: Gly, Ala, Val, Leu, Ile, Ser, and Thr (representing aliphatic and aliphatic hydroxyl side chains); Class III: Tyr, Ser, and Thr (representing hydroxyl side chains); Class IV: Cys and Met (representing sulfur-containing side chains); Class V: Glu, Asp, Asn and Gln (carboxyl or amide group containing side chains); Class VI: His, Arg and Lys (representing basic side chains); Class VII: Gly, Ala, Pro, Trp, Tyr, Ile, Val, Leu, Phe and Met (representing hydrophobic side chains); Class VIII: Phe, Trp, and Tyr (representing aromatic side chains); and Class IX: Asn and Gln (representing amide side chains). The classes are not limited to naturally occurring amino acids, but also include artificial amino acids, such as beta or gamma amino acids and those containing non-natural side chains, and/or other similar monomers such as hydroxyacids.

SEQ ID NO:1 is shown in FIG. 1 in a multiple protein alignment with three other proteins having MLL lactonase activity. Identical amino acids are marked with an asterisk. Conservative amino acids with strongly similar properties and weakly similar properties are marked with a colon and a period, respectively.

Guidance concerning how to make phenotypically silent amino acid substitutions is provided in Bowie et al. (1990, Science, 247:1306-1310), wherein the authors indicate proteins are surprisingly tolerant of amino acid substitutions. For example, Bowie et al. disclose that there are two main approaches for studying the tolerance of a polypeptide sequence to change. The first method relies on the process of evolution, in which mutations are either accepted or rejected by natural selection. The second approach uses genetic engineering to introduce amino acid changes (substitutions) at specific positions of a cloned gene and selects or screens to identify sequences that maintain functionality. As stated by the authors, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al, and the references cited therein.

A MLL lactonase protein described herein includes one or more amino acid substitutions in comparison to a reference MLL lactonase protein. In one embodiment, the reference protein is SEQ ID NO:1, and the substitution is present at one or more of M21, W25, Q41, F47, S66, S81, T82, M85, F86, T91, R111, L120, F141, A144, C147, E154, A156, G155, V175, H178, I182, L183, Y222, I237, M244, or N245. In one embodiment, the substitution is for any other amino acid, e.g., the substitution at position 21 can be to any amino acid other than methionine. In one embodiment, the substitution is for a conservative amino acid, e.g., the substitution at position 21 can be to the Class IV amino acid Cys (a sulfur-containing side chain) or to a Class VII amino acid Gly, Ala, Pro, Trp, Tyr, Ile, Val, Leu, or Phe (a hydrophobic side chain). In one embodiment, Q41 is substituted with a P, S66 is substituted with an A, S81 is substituted with an A, T91 is substituted with a S, R111 is substituted with a K, A144 is substituted with a T, C147 is substituted with a S, V175 is substituted with a I, H178 is substituted with a D, 1182 is substituted with a L, L183 is substituted with a E, M244 is substituted with a A, or N245 is substituted with a K. The MLL lactonase protein can include 1, 2, 3, 4, 5, 6, 7, 8, 9, 10, 11, 12, 13, 14, 15, 16, 17, 18, 19, 20, 21, 22, 23, 24, 25, or all 26 of these mutations, in any combination. In one embodiment, a MLL lactonase protein includes the C147S, H178D, L183E, and M244A mutations. In another embodiment, a MLL lactonase protein includes the A156R or A156Y mutation, and has increased specificity for lactones with shorter alkyl chains. In another embodiment, a MLL lactonase protein includes all 26 of the substitutions.

While the specific residues of a MLL lactonase protein described herein are based on the numbering of the enzyme depicted at SEQ ID NO:1, other MLL lactonase proteins can have the same substitution at a functionally equivalent residue. As used herein, “functionally equivalent” and “functional equivalent” refers to an amino acid position in a lactonase protein that occurs at a position having the same functional role as that amino acid position in a reference protein. A functionally equivalent amino acid position in a MLL lactonase protein occurs at a position having the same functional role as that amino acid position in a reference protein such as the enzyme depicted at SEQ ID NO:1.

Functionally equivalent substitution mutations in different MLL lactonase proteins occur at homologous amino acid positions in the amino acid sequences of the enzymes. Functionally equivalent amino acid residues in the amino acid sequences of two or more different MLL lactonase proteins can be easily identified by the skilled person on the basis of sequence alignment. An example of sequence alignment to identify functionally equivalent residues is set forth in FIG. 1. The corresponding residues in the MLL lactonase proteins from Parageobacillus caldoxylosilyticus (Gcl, SEQ ID NO: 1), Alicyclobacter acidoterrestris, (AaL, SEQ ID NO:2), Bacillus thuringiensis (AiiA, SEQ ID NO:3) and Agrobacterium tumefaciens (AiiB, SEQ ID NO:4) are identified in the Figure as vertically aligned and are considered positionally equivalent as well as functionally equivalent to the corresponding residue in the MLL lactonase protein amino acid sequence of SEQ ID NO:1.

A MLL lactonase protein described herein includes at least one altered characteristic compared to a reference MLL lactonase protein. In one embodiment, MLL lactonase protein described herein can have reduced thermal stability. A MLL lactonase protein described herein can have a melting temperature that is decreased by at least 18° C., at least 19° C., at least 20° C., at least 21° C., or at least 22° C.

In one embodiment, a MLL lactonase protein described herein can have increased catalytic activity compared to a reference MLL lactonase protein. A MLL lactonase protein described herein can have a catalytic efficiency (k_{cat}/K_{M}) that is increased by at least one factor of 10 (one order of magnitude), at least two factors of 10 (two orders of magnitude), or at least three factors of 10 (three orders of magnitude) for substrates C4-AHL, C8-AHL, 3-oxo-C8 AHL, γ-Butyrolactone, γ-Decanolactone, δ-Valerolactone, and ε-Caprolactone compared to the reference MLL lactonase protein Gdl (SEQ ID NO:1).

In one embodiment, a MLL lactonase protein described herein can have increased substrate specificity compared to a reference MLL lactonase protein. A MLL lactonase protein described herein can have a catalytic efficiency (k_{cat}/K_{M}) that is increased or decreased by at least one factor of 10 (one order of magnitude), at least two factors of 10 (two orders of magnitude), or at least three factors of 10 (three orders of magnitude) for at least one of the substrates C4-AHL, C8-AHL, 3-oxo-C8 AHL, γ-Butyrolactone, γ-Decanolactone, δ-Valerolactone, and ε-Caprolactone compared to the reference MLL lactonase protein Gdl (SEQ ID NO:1).

### PLL Proteins

In one embodiment, a protein that catalytically alters the structure of an AHL is referred to herein as a PLL lactonase. Whether a protein has PLL lactonase activity can be determined by in vitro assays. In one embodiment, an in vitro assay is carried out by using a pH indicator assay as described in the Examples. Briefly, a lactone hydrolysis assay can be performed in lactonase buffer (Bicine 2.5 mM pH 8.3, NaCl 150 mM, CoCl2 0.2 mM, Cresol purple 0.25 mM and 0.5% DMSO). The cresol purple (pKₐ 8.3 at 25° C.) is a pH indicator following the lactone ring hydrolysis by media acidification (molar extinction coefficient at ε577 nm=2 923 M⁻¹ cm⁻¹). In one embodiment, the substrate is C12-AHL.

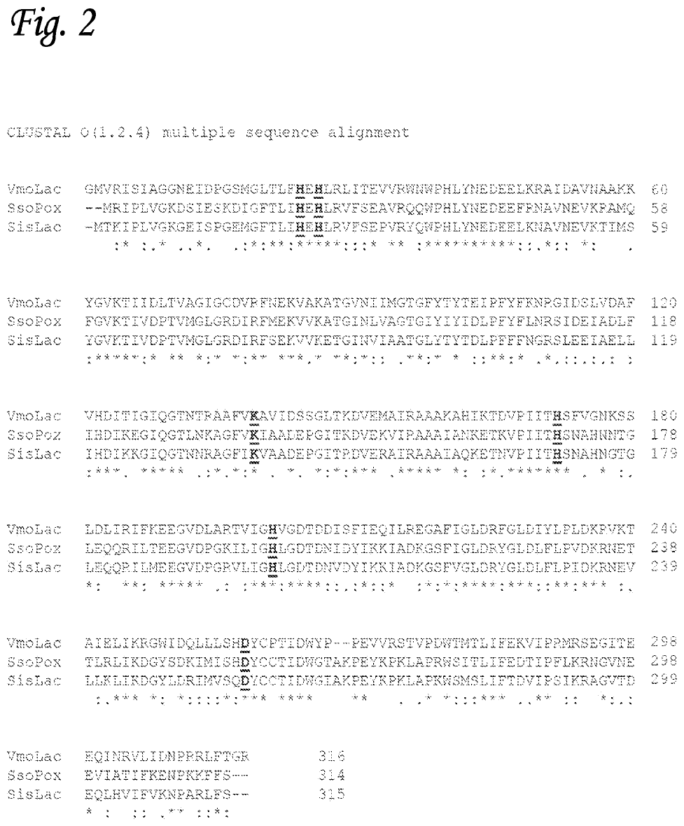

A PLL lactonase described herein is a member of the phosphotriesterase-like lactonases (PLL) family. The PLL family exhibit a conserved set of amino acids involved in the binding of two metal cations used in catalysis (Elias and Tawfik, 2012, J Biol Chem, 287:11-20). Those amino acids are shown in FIG. 2 as bold underlined residues. Examples of PLL lactonase proteins are depicted at SEQ ID NOs:5, 6, and 7.

A PLL lactonase protein described herein includes one or more amino acid substitutions in comparison to a reference PLL lactonase protein. The amino acid mutations are described herein. Other examples of PLL lactonase proteins of the present disclosure include those having structural similarity with the amino acid sequence of SEQ ID NO: 5, 6, or 7. A lactonase protein having structural similarity with the amino acid sequence of SEQ ID NO:5, 6, or 7 has PLL lactonase activity. A PLL lactonase protein can be isolated from a microbe, may be produced using recombinant techniques, or chemically or enzymatically synthesized using routine methods. Methods for determining whether a protein has structural similarity with the amino acid sequence of SEQ ID NO:5, 6, or 7 are described herein.

The amino acid sequence of a PLL lactonase protein having structural similarity to SEQ ID NO:5, 6, or 7 can include conservative substitutions of amino acids present in SEQ ID NO: 5, 6, or 7. Conservative substitutions are described herein.

SEQ ID NO:5 is shown in FIG. 2 in a multiple protein alignment with two other proteins having PLL lactonase activity. Identical amino acids are marked with an asterisk. Conservative amino acids with strongly similar properties and weakly similar properties are marked with a colon and a period, respectively.

A PLL lactonase protein described herein includes one or more amino acid substitutions in comparison to a reference PLL lactonase protein. In one embodiment, the reference protein is SEQ ID NO:5, and the substitution is present at one or more of R2, S10, S13, K14, D15, 116, R55, Q58, F59, L90, V91, G93, I100, L107, L130, I138, N160, T186, or R241. In one embodiment, the substitution is for any other amino acid, e.g., the substitution at position 2 can be to any amino acid other than an arginine. In one embodiment, the substitution is for a conservative amino acid, e.g., the substitution at position 2 can be to a Class VI amino acid His or Lys (a basic side chain). In one embodiment, R2 is substituted with a K, S10 is substituted with a E, S13 is substituted with a P, K14 is substituted with a R, D15 is substituted with a E, 116 is substituted with a M, R55 is substituted with a T, Q58 is substituted with a S, F59 is substituted with a Y, L90 is substituted with a V, V91 is substituted with a I, G93 is substituted with a A, I100 is substituted with a T, L107 is substituted with a N, L130 is substituted with a N, I138 is substituted with a V, N160 is substituted with a H, T186 is substituted with a M, or R241 is substituted with a K. The PLL lactonase protein can include 1, 2, 3, 4, 5, 6, 7, 8, 9, 10, 11, 12, 13, 14, 15, 16, 17, 18, or all 19 of these substitutions, in any combination. In one embodiment, a PLL lactonase protein includes the R2K, S10E, S13P, K14R, V91I, and L107N mutations. In another embodiment, a PLL lactonase protein includes all 19 of the mutations.

While the specific residues of a PLL lactonase protein described herein are based on the numbering of the enzyme depicted at SEQ ID NO:5, other PLL lactonase proteins can have the same substitution at a functionally equivalent residue. A functionally equivalent amino acid position in a PLL lactonase protein occurs at a position having the same functional role as that amino acid position in a reference protein such as the enzyme depicted at SEQ ID NO:5.

Functionally equivalent substitution mutations in different PLL lactonase proteins occur at homologous amino acid positions in the amino acid sequences of the enzymes. Functionally equivalent amino acid residues in the amino acid sequences of two or more different PLL lactonase proteins can be easily identified by the skilled person on the basis of sequence alignment. An example of sequence alignment to identify functionally equivalent residues is set forth in FIG. 2. The corresponding residues in the PLL lactonase proteins from Sulfolobus solfataricus (SsoPox, SEQ ID NO:5), Sulfolobus islandicus (SisLac, SEQ ID NO:6), and Vulcanisaeta moutnovskia (VmoLac, SEQ ID NO:7) are identified in the Figure as vertically aligned and are considered positionally equivalent as well as functionally equivalent to the corresponding residue in the PLL lactonase protein amino acid sequence of SEQ ID NO:5.

A PLL lactonase protein described herein includes at least one altered characteristic compared to a reference MLL lactonase protein. In one embodiment, MLL lactonase protein described herein can have increased thermal stability. A PLL lactonase protein described herein activity that is increased compared to a wild type PLL protein, such as SEQ ID NO:5, after incubation at an increased temperature.

In one embodiment, PLL lactonase protein described herein can have increased yield when expressed in a host cell such as E. coli compared to a wild type PLL protein isolated under the same conditions.

### Structural Similarity

Whether a MLL protein is structurally similar to a protein of SEQ ID NO:1, 2, 3, or 4, or a PLL protein is structurally similar to a protein of SEQ ID NO:5, 6, or 7 can be determined by aligning the residues of the two proteins (for example, a candidate protein and any appropriate reference protein described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference protein may be a protein described herein. In one embodiment, a reference protein is a protein described at SEQ ID NO:1, 2, 3, or 4. In another embodiment, a reference protein is a protein described at SEQ ID NO:5, 6, or 7. A candidate protein is the protein being compared to the reference protein. A candidate protein can be produced using recombinant techniques, or chemically or enzymatically synthesized.

Unless modified as otherwise described herein, a pair-wise comparison analysis of amino acid sequences can be carried out using the Blastp program of the Blastp suite-2sequences search algorithm, as described by Tatusova et al., (FEMS Microbiol Lett, 174, 247-250 (1999)), and available on the National Center for Biotechnology Information (NCBI) website. The default values for all blastp suite-2sequences search parameters may be used, including general paramters: expect threshold=10, word size=3, short queries=on; scoring parameters: matrix=BLOSUM62, gap costs=existence:11 extension:1, compositional adjustments=conditional compositional score matrix adjustment. Alternatively, proteins may be compared using other commercially available algorithms, such as the BESTFIT algorithm in the GCG package (version 10.2, Madison Wis.).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions.

Thus, as used herein, reference to an amino acid sequence disclosed at SEQ ID NO:1, 2, 3, 4, 5, 6, or 7 can include a protein with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference amino acid sequence.

Alternatively, as used herein, reference to an amino acid sequence disclosed at SEQ ID NO:1, 2, 3, 4, 5, 6, or 7 can include a protein with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

### Further Aspects of the Proteins

The present disclosure also includes fragments of the proteins described herein, and the polynucleotides encoding such fragments, such as SEQ ID NOs:1 and 5, respectively, as well as those polypeptides having structural similarity to, for instance, SEQ ID NO: 1 or SEQ ID NO:5. A protein fragment may include a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acid residues.

A protein described herein can be expressed as a fusion protein that includes a MLL lactonase or a PLL lactonase protein and an additional amino acid sequence. For instance, the additional amino acid sequence may be useful for purification of the fusion protein by affinity chromatography. Various methods are available for the addition of such affinity purification moieties to proteins. Representative examples may be found in Hopp et al. (U.S. Pat. No. 4,703,004), Hopp et al. (U.S. Pat. No. 4,782,137), Sgarlato (U.S. Pat. No. 5,935,824), and Sharma Sgarlato (U.S. Pat. No. 5,594,115).

### Polynucleotides

The present disclosure also includes isolated polynucleotides encoding a protein described herein, e.g., a MLL lactonase or a PLL lactonase. A polynucleotide encoding a protein described herein is referred to as a MLL lactonase polynucleotide or a PLL lactonase polynucleotide. A MLL lactonase polynucleotides can have a nucleotide sequence encoding a protein having the amino acid sequence shown in, e.g., SEQ ID NO:1 with one or more of the mutations described herein, and a PLL lactonase polynucleotide can have a nucleotide sequence encoding a protein having the amino acid sequence shown in, e.g., SEQ ID NO:5 with one or more of the substitutions s described herein. A nucleotide sequence of a polynucleotide encoding a protein described herein can be readily determined by one skilled in the art by reference to the standard genetic code, where different nucleotide triplets (codons) are known to encode a specific amino acid. As is readily apparent to a skilled person, the class of nucleotide sequences that encode any protein described herein is large as a result of the degeneracy of the genetic code, but it is also finite.

A polynucleotide encoding a protein described herein can be present in a vector. A vector is a replicating polynucleotide, such as a plasmid, phage, or cosmid, to which another polynucleotide may be attached so as to bring about the replication of the attached polynucleotide. Construction of vectors containing a polynucleotide employs standard ligation techniques known in the art. See, e.g., Sambrook et al, Molecular Cloning: A Laboratory Manual., Cold Spring Harbor Laboratory Press (1989). A vector may provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, and artificial chromosome vectors. Examples of viral vectors include, for instance, adenoviral vectors, adeno-associated viral vectors, lentiviral vectors, retroviral vectors, and herpes virus vectors. Typically, a vector is capable of replication in a microbial host, for instance, a prokaryotic bacterium, such as E. coli. Preferably the vector is a plasmid.

Selection of a vector depends upon a variety of desired characteristics in the resulting construct, such as a selection marker, vector replication rate, and the like. In some aspects, suitable host cells for cloning or expressing the vectors herein include eukaryotic cells. Suitable host cells for cloning or expressing the vectors herein include prokaryotic cells. Suitable prokaryotic cells include eubacteria, such as gram-negative microbes, for example, E. coli. Vectors may be introduced into a host cell using methods that are known and used routinely by the skilled person. For example, calcium phosphate precipitation, electroporation, heat shock, lipofection, microinjection, and viral-mediated nucleic acid transfer are common methods for introducing nucleic acids into host cells.

Polynucleotides can be produced in vitro or in vivo. For instance, methods for in vitro synthesis include, but are not limited to, chemical synthesis with a conventional DNA/RNA synthesizer. Commercial suppliers of synthetic polynucleotides and reagents for such synthesis are well known.

An expression vector optionally includes regulatory sequences operably linked to the coding region. The disclosure is not limited by the use of any particular promoter, and a wide variety of promoters are known. Promoters act as regulatory signals that bind RNA polymerase in a cell to initiate transcription of a downstream (3′ direction) coding region. The promoter used may be a constitutive or an inducible promoter. It may be, but need not be, heterologous with respect to the host cell.

An expression vector may optionally include a ribosome binding site and a start site (e.g., the codon ATG) to initiate translation of the transcribed message to produce the polypeptide. It may also include a termination sequence to end translation. A termination sequence is typically a codon for which there exists no corresponding aminoacetyl-tRNA, thus ending polypeptide synthesis. The polynucleotide used to transform the host cell may optionally further include a transcription termination sequence.

A vector introduced into a host cell optionally includes one or more marker sequences, which typically encode a molecule that inactivates or otherwise detects or is detected by a compound in the growth medium. For example, the inclusion of a marker sequence may render the transformed cell resistant to an antibiotic, or it may confer compound-specific metabolism on the transformed cell. Examples of a marker sequence are sequences that confer resistance to kanamycin, ampicillin, chloramphenicol, tetracycline, and neomycin.

Proteins described herein may be produced using recombinant DNA techniques, such as an expression vector present in a cell. Such methods are routine and known in the art. A protein can also be synthesized in vitro, e.g., by solid phase peptide synthetic methods. The solid phase peptide synthetic methods are routine and known in the art. A protein produced using recombinant techniques or by solid phase peptide synthetic methods may be further purified by routine methods, such as fractionation on immunoaffinity or ion-exchange columns, ethanol precipitation, reverse phase HPLC, chromatography on silica or on an anion-exchange resin such as DEAE, chromatofocusing, SDS-PAGE, ammonium sulfate precipitation, gel filtration using, for example, Sephadex G-75, or ligand affinity.

### Genetically Modified Organisms

The present disclosure also includes genetically modified organisms that have an exogenous polynucleotide encoding a MLL lactonase or a PLL lactonase described herein. As used herein, “genetically modified organism” refers to an organism into which has been introduced an exogenous polynucleotide. Examples of organisms include, for instance, microbes, plants, and animals. For example, a microbe is a genetically modified organism by virtue of introduction into a suitable microbe of an exogenous polynucleotide, and a plant is a genetically modified organism by virtue of introduction into a suitable plant cell of an exogenous polynucleotide and generation of a transgenic plant from the plant cell. Compared to a control organism that is not genetically modified, a genetically modified organism can exhibit production of a MLL lactonase or a PLL lactonase described herein. A polynucleotide encoding a MLL lactonase or a PLL lactonase can be present in the organism as a vector or integrated into a chromosome.

The microbial host can be a member of the domain Bacteria or a member of the domain Archaea. In one embodiment, the bacterial host cell can be an extremophile, including but not limited to, an anaerobe, halophile, thermophile, hyperthermophile, oligotroph, or psychrophile.

Examples of useful microbial host cells include, but are not limited to, Escherichia (such as Escherichia coli), Pichia, or Bacillus.

The plant can be a horticultural and a crop plant. Examples include monocotyledons (such as, but not limited to, corn, wheat, and barley) and dicotyledons (such as, but not limited to, soybean, beans, potato, tomatoes).

### Compositions

Also provided by the disclosure are compositions that include a protein described herein. In one embodiment, a composition can include a solvent. A solvent can be aqueous or organic. Proteins described herein are surprisingly resistant to organic solvents. Examples of organic solvents include, but are not limited to, acetone, acetonitrile, butanone, butyl acetate, chloroform, dichloromethane, diethyl ether, ethanol, ethyl acetate, isopropanol, methanol, methoxypropanol, petroleum ether, toluene, and xylene. An example of an aqueous solvent is a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration.

A composition can include an agent to aid in stability of the protein and/or ability to remain associated with a surface, such as a surface of a plant or an article. For instance, a composition can include other agents to aid in the application of a protein including, but not limited to, a surfactant (for instance, anionic, cationic, amphoteric, nonionic), a biosurfactant, a wetting agent, a penetrant, a thickener, an emulsifier, a spreader, a sticker, an oil, an alkyl polyglucoside, an organosilicate, an inorganic salts, or a combination thereof.

A composition that includes a pharmaceutically acceptable carrier can be prepared by methods well known in the art of pharmaceutics. In general, a composition can be formulated to be compatible with its intended route of administration. Administration may be systemic or local. In some aspects local administration may have advantages for site-specific, targeted disease management. Local therapies may provide high, clinically effective concentrations directly to the treatment site, with less likelihood of causing systemic side effects. Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. Appropriate dosage forms for enteral administration of the protein described herein can include tablets, capsules or liquids. Appropriate dosage forms for parenteral administration may include intravenous administration. Appropriate dosage forms for topical administration may include nasal sprays, metered dose inhalers, dry-powder inhalers or by nebulization. Other dosage forms for topical administration may include cosmetic formulations such as skin treatments (e.g., antimicrobial ointment), acne treatments (e.g., anti-acne ointment), toothpaste, and mouth rinse formulations.

In one embodiment, a composition is formulated for use as a coating. A coating can be used to cover a surface, can be incorporated, e.g., impregnated, into a surface, or a combination thereof. A protein described herein can be combined with agents suitable for use in coating a surface, such as, but not limited to, polymers, plasticizers, pigments, colorants, glidants, stabilization agents, pore formers, and/or surfactants. Examples of polymers useful as coating agents include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, polyurethane, epoxy, acrylic acid polymers and copolymers, and methacrylic resins, zein, shellac, and polysaccharides.

In one embodiment, a composition is formulated for use as a cleaning solution. Such a composition is suitable for application to a surface for cleaning and/or disinfecting the surface. A protein described herein can be formulated into a solution in a suitable solvent for administration in a spray bottle, for use as an aerosol or a foam suitable for applying onto surfaces. In one embodiment, a formulation for use as a cleaning/disinfecting solution includes, in addition to one or more proteins described herein, an acceptable carrier and an antimicrobial agent. In one embodiment, a formulation for use as a cleaning/disinfecting solution includes, in addition to one or more proteins described herein, a cleaning agent, a disinfecting agent, or a combination thereof. An antimicrobial agent can be microbiocidal or microbiostatic. Antimicrobial agents that can be incorporated into cleaning formulations are known in the art. Methods for making formulations for use as a disinfectant are known in the art.

Examples of surfaces that can be coated and/or disinfected with a composition described herein can be biological or non-biotic. Examples of biological surfaces include, but are not limited to, a surface of an animal or a plant. Examples of non-biotic surfaces include any medium, e.g., plastic, glass, or metal, used in an article, for instance, prosthetics, floors, counters, soil, dental instruments, teeth, dentures, dental retainers, dental braces including plastic braces, medical instruments, medical devices (e.g., endoscope), contact lenses and lens cases, catheters, bandages, tissue dressings, surfaces (e.g., tabletop, countertop, bathtub, tile, filters (e.g., water filter), membranes (e.g., reverse osmosis membrane, etc.), fabrics (e.g., anti-odor fabric), tubing, drains, pipes including water pipes, gas pipes, oil pipes, drilling pipes, fracking pipes, sewage pipes, drainage pipes, hoses, fish tanks, showers, children's toys, boat hulls, cooling- and heating-water systems including cooling towers, and surfaces used for testing such as test coupons.

In one embodiment, a composition is formulated for use as a feed supplement or a dietary supplement.

Also provided is a surface, such as an article, that includes a protein described herein. For instance, the surface can include the protein as a coating, impregnated therein, or a combination thereof.

A composition can be used alone or in combination with one or more other agents such as, but not limited to, s anti-microbial, bactericidal, bacteriostatic, anti-viral, or anti-fungal compounds.

### Methods of Use

Lactones are often used by microbes for communication that can result in, for instance, the coordination of actions in a cell density-dependent manner. Such communication causes changes in gene expression and results in, for instance, biofilm formation or increased virulence. In general, the methods described herein include the use of lactonases to enzymatically degrade lactones, disrupting the ability of microbes to communicate, thereby reducing the coordination of actions between microbes.

Biofilms

As used herein, a “biofilm” refers to a community of microbes that stick to each other and to a surface. In one embodiment, a method is for preventing biofilm formation or buildup on a surface. Preventing biofilm formation includes preventing the creation of a biofilm on a surface. Preventing biofilm buildup includes preventing or reducing the expansion or growth or increase in size of a biofilm that is present on a surface before treating with a lactonase. Biofilm formation typically begins with the attachment of a microbe to a surface. The first microbes adhere to the surface initially through weak adhesive forces. The microbes can subsequently anchor themselves more permanently using adhesion structures produced by the cells. Some species are not able to attach to a surface on their own but are sometimes able to anchor themselves to the matrix or directly to microbes that have already colonized a surface. During this colonization the cells communicate via quorum sensing, and, without intending to limiting, it is this quorum sensing that is disrupted using a protein described herein. After colonization begins, the biofilm grows through a combination of cell division and recruitment. Polysaccharide matrices typically enclose bacterial biofilms. In one embodiment, a biofilm can be made up of one species of microbial cell. In other embodiments, a biofilm can include more than one species of microbial cell, for instance at least 2, 3, 4, 5, 6, 7, 8, 9, 10, 15, 20, 30, 40, 50, 60, 70, 80, 90, 100 or more different species of microbial cell. Microbial species in a biofilm can include microbes that are gram-negative, gram-positive, aerobic, anaerobic, or a combination thereof.

In one embodiment, the method includes treating an existing biofilm with a lactonase described herein. In another embodiment, the method includes treating a surface that is at risk for biofilm formation. A surface that is at risk for biofilm formation includes, but is not limited to, a surface that is free of a biofilm but is exposed to, or will be exposed to, conditions suitable for biofilm formation. As used herein, “treating” includes, but is not limited to, touching, impregnating, mixing, integrating, coating, spraying, dipping, flushing, irrigating, and wiping. In one embodiment, the treating can include applying a lactonase on, or in the vicinity of, a biofilm. In certain embodiments, it may be desirable to provide continuous delivery of one or more lactonases to the surface being treated. In this aspect of the disclosure, an “effective amount” is an amount effective in inhibiting biofilm formation or buildup on a surface or reducing or removing biofilm from a surface.

Biofilms that are targeted with this method can be produced by or include microbes that use lactones for communication such as, but not limited to, Acinetobacter baumannii, Escherichia coli, Pseudomonas aeruginosa, Pseudomonas putida, Streptococcus mutans, Salmonella enterica, Pectobacterium carotovorum, Xanthomonas translucens pv. Translucens, Xanthomonas translucens pv. undulosa LMG892, Clavibacter michiganensis subsp. Nebraskensis, Pseudomonas syringae pv. Syringae, Acinetobacter sp., Aeromonas sp., Agrobacterium sp., Burkholderia sp., Dickeya sp., Erwinia sp., Proteus sp., Pectobacterium sp., Xanthomonas sp., Pseudomonas sp., Ralstonia sp., Serratia sp., Vibrio sp., Streptomyces sp., and Rhodococcus sp. Biofilms that are targeted with this method can be produced by or include microbes that may use other molecules that respond directly or indirectly to lactonases. Examples of such microbes include, but are not limited to, Clavibacter sp., Streptococcus sp., Salmonella sp., and E. coli.

In one embodiment, treating a biofilm can result in removal of a biofilm from a surface. In another embodiment treating a biofilm can result in reducing or preventing the effect a biofilm can have, e.g., effects such as bioclogging, biocorrosion, reduction of heat transfer, spread of invasive species, or biofouling. The method can allow inhibition or prevention of biofilm formation on the surfaces being contacted, and optionally reduction of transmission of biofilm forming microorganisms from the surface to another surface. In some embodiments, the number of the bacterial colony forming units on the surface being contacted with a lactonase may be reduced by at least 10%, at least 20%, at least 30%, at least 40%, at least 50%, at least 60%, at least 70%, at least 80%, at least 90%, or 100% compared to the number of colony forming units on the surface immediately before treating with the lactonase.

A surface can be biological or non-biotic. Examples of biological surfaces include, but are not limited to, a surface of an animal or a plant. Examples of non-biotic surfaces include any medium, e.g., plastic, glass, or metal, used in an article, for instance, prosthetics, floors, counters, soil, dental instruments, teeth, dentures, dental retainers, dental braces including plastic braces, medical instruments, medical devices (e.g., endoscope), contact lenses and lens cases, catheters, bandages, tissue dressings, surfaces (e.g., tabletop, countertop, bathtub, tile, filters (e.g., water filter), membranes (e.g., reverse osmosis membrane, etc.), fabrics (e.g., anti-odor fabric), tubing, drains, pipes including water pipes, gas pipes, oil pipes, drilling pipes, fracking pipes, sewage pipes, drainage pipes, hoses, fish tanks, showers, children's toys, boat hulls, cooling- and heating-water systems including cooling towers, and surfaces used for testing such as test coupons.

Disinfectant

In one embodiment, a composition can be used to aid in disinfecting a surface or keeping a surface disinfected. In one embodiment, the method includes treating a surface with a composition that includes one or more proteins described herein and an antimicrobial, antiviral, or antifungal agent. In one embodiment, the surface is one that is at risk for biofilm formation. Without intending to be limited by theory, it is expected that a protein described herein can increase the effectiveness of an antimicrobial, antiviral, and/or antifungal agent.

Infection

In one embodiment, a method is for treating an infection in a subject. As used herein, the term “infection” refers to the presence of and multiplication of a pathogen (e.g., microbe, virus, or fungus) in or on the body of a subject.

Animals

In one embodiment, the method includes administering to an animal an effective amount of a composition that includes a protein described herein. Examples of animals that can be treated include humans, murine (mice and rats), domesticated livestock such as bovine, porcine, equine, and avian species. The treatment of an animal can result in a reduction in the amount of the pathogen (e.g., the number of colony forming units (cfu)) in or on the body of the subject. The reduction can be at least 5%, at least 10%, at least 20%, at least 30%, at least 40%, at least 50%, at least 60%, at least 70%, at least 80%, or at least 90% compared to the subject before the administration. In this aspect, the term “effective amount” refers to an amount that is sufficient to result in the desired effect of reducing the amount of the pathogen.

The bacterium causing the infection can be one that uses a chemical signaling system that includes acyl homoserine lactones (AHLs) to coordinate virulence factor expression. Examples of animal diseases or conditions that can be caused by a pathogen having its pathogenicity controlled by quorum sensing include, but are not limited to, impetigo, boils, abscesses, folliculitis, cellulitis, necrotizing fasciitis, pyomyositis, surgical/traumatic wound infection, and infected ulcers and burns, osteomyelitis, device-related osteoarticular infections, secondarily infected skin lesions, meningitis, brain abscess, subdural empyema, spinal epidural abscess, arterial damage, gastritis, urinary tract infections, biliary tract infections, pyelonephritis, cystitis, sinus infections, ear infections, otitis media, otitis externa, leprosy, tuberculosis, conjunctivitis, bloodstream infections, benign prostatic hyperplasia, chronic prostatitis, lung infections including chronic lung infections of humans with cystic fibrosis, osteomyelitis, catheter infections, bloodstream infections, skin infections, acne, rosacea, dental caries, periodontitis, gingivitis, nosocomial infections, arterial damage, endocarditis, periprosthetic joint infections, open or chronic wound infections, venous stasis ulcers, diabetic ulcers, arterial leg ulcers, pressure ulcers, endocarditis, pneumonia, orthopedic prosthesis and orthopedic implant infections, peritoneal dialysis peritonitis, cirrhosis, and other acute or chronic infections of an animal.

Examples of microbes with pathogenicity controlled by quorum sensing and causing disease in an animal include, but are not limited to, Acinetobacter baumannii, Aeromonas spp., Burkholderia spp., Burkholderia cepacia, Burkholderia cenocepacia, Burkholderia pseudomallei, Escherichia coli (EHEC) O157:H7, Klebsiella spp., Pseudomonas aeruginosa, Salmonella spp., Staphylococcus spp., Staphylococcus sciuri, Streptococcus pyogenes, Vibrio spp., and Yersinia enterocolitica.

The animal can have or be at risk of having an infection or displaying a clinical sign of a condition caused by infection by a pathogen. Treatment of an infection or clinical signs associated with an infection can be prophylactic or, alternatively, can be initiated after the development of an infection or clinical sign described herein. Clinical signs associated with conditions referred to herein and the evaluations of such signs are routine and known in the art. Treatment that is prophylactic, for instance, initiated before a subject manifests signs of an infection or clinical sign caused by a pathogen, is referred to herein as treatment of a subject that is “at risk.” Typically, a subject “at risk” is a subject present in an area where subjects having the condition have been diagnosed and/or are likely to be exposed to a pathogen causing the condition. Accordingly, administration of a composition can be performed before, during, or after the occurrence of the conditions described herein. Treatment initiated after the development of a condition may result in decreasing the severity of the signs of one of the conditions, or completely removing the signs. In this aspect of the disclosure, an “effective amount” is an amount effective to prevent the manifestation of signs of a disease, decrease the severity of the signs of a disease, and/or completely remove the signs. Such a dosage can be easily determined by the skilled person.

The types of infections that can be treated include, but are not limited to, those whose pathologies include colonization of a surface, such as an exterior surface or an interior surface. Examples of an exterior surface include but are not limited to skin or exterior mucus membrane (e.g., eye, ear) of an animal. An interior surface includes mucus membrane surfaces of an animal that are contiguous with the outside environment but are internal, such as an oral cavity, respiratory passages, and gut passages.

The administration can be by any method that results in exposing the pathogen to one or more proteins described herein. For instance, if the infection is topical the skin of the animal can be contacted with a composition that includes the protein, a nebulizer can be used to administer the composition to a mucosal membrane of the respiratory tract, or parenteral administration can be used. The composition administered can include an antimicrobial agent, antiviral, and/or antifungal agent. Optionally, the treatment can include separate administration of an antimicrobial agent, antiviral, and/or antifungal agent.

Also provided by the present disclosure are animals that include a protein described herein. The protein can be present systemically or on a part of the animal. Also provided herein are methods for applying a protein to an animal, or a part of the animal, with a composition that includes a protein described herein.

Plants

In another embodiment, the method includes administering to a plant an effective amount of a composition that includes a protein described herein. Examples of plants that can be treated include monocotyledons (such as, but not limited to, corn, wheat, and barley) and dicotyledons (such as, but not limited to, soybean, beans, potato). The treatment of a plant results in a reduction in the amount (e.g., the number of colony forming units (cfu)) of the microbe, virus, and/or fungus in or on the body of the plant. The reduction can be at least 5%, at least 10%, at least 20%, at least 30%, at least 40%, at least 50%, at least 60%, at least 70%, at least 80%, or at least 90% compared to the subject before the administration. In this aspect, the term “effective amount” refers to an amount that is sufficient to result in the desired effect of reducing the amount of the pathogen.

The bacterium causing the infection can be one that uses a chemical signaling system that includes acyl homoserine lactones (AHLs) to coordinate virulence factor expression. The bacterium also may produce other molecules that respond directly or indirectly with lactonases to limit plant disease. Examples of plant diseases or conditions that can be caused by a pathogen having its pathogenicity controlled by quorum sensing include, but are not limited to, Citrus Canker, Pierce's Disease of grapes, Bacterial Speck, Bacterial Canker, Pith Necrosis, Bacterial Wilt and Bacterial Spot of plants such as peppers, tomatoes, potatoes, wheat, and other horticultural and crop plants, and other acute or chronic infection of a plant.

Examples of microbes with pathogenicity controlled by quorum sensing and causing disease in a plant include, but are not limited to, Agrobacterium spp., Agrobacterium tumefaciens, Agrobacterium rhizogenes, Burkholderia spp., Dickeya spp., Erwinia spp., Erwinia toletana, Clavibacter michiganensis subsp. Nebraskensis, Pantoea ananatis, Pantoea stewartii subsp. stewartii, Pectobacterium spp., Pectobacterium carotovorum ssp. Atrosepticum, Pectobacterium carotovorum ssp. Carotovorum, Pectobacterium chrysanthemi, Pseudomonas syringae pv. Syringae, Pseudomonas savastanoi pv. savastanoi, P. syringae pv. tabaci, Pseudomonas spp., Ralstonia solanacearum, Serratia liquefaciens, Xanthomonas spp., Xanthomonas axonopodis, X campestris pv. campestris, Xanthomonas translucens pv. Translucens, Xanthomonas oryzae, Xanthomonas translucens pv. undulosa LMG892.

Examples of microbes that may use other molecules that respond directly or indirectly to lactonases to limit plant disease include, but are not limited to, Clavibacter sp., Streptococcus sp., Salmonella sp., and E. coli.

Treatment of a plant can be prophylactic or, alternatively, can be initiated after the development of disease caused by a pathogen. Treatment that is prophylactic, for instance, initiated before a plant manifests signs of disease, is referred to herein as treatment of a plant that is “at risk” of having an infection. Treatment can be performed before, during, or after the occurrence of an infection by a pathogen. Treatment initiated before the development of disease may result in decreasing the risk of infection by the pathogen. Treatment initiated before development of disease includes applying a protein described herein to the surface of a plant, such as a leaf or stalk. Treatment initiated after the development of disease may result in decreasing the severity of the signs of the disease, or completely removing the signs. Signs of disease in a plant by a pathogen vary depending upon the pathogen and are known in the art. The dosage administered to a plant is sufficient to result in decreased risk of infection or decreased severity of the signs of the disease. Decreased risk of infection or decreased severity of the signs of the disease can be the result of reduced growth of the pathogen. Such a dosage can be easily determined by the skilled person.

The types of infections that can be treated include, but are not limited to, those whose pathologies include colonization of a surface, such as an exterior surface. Examples of an exterior surface include but are not limited to a leaf or stalk of a plant.

The administration can be by any method that results in exposing the pathogen or plant part to one or more proteins described herein. In one embodiment, the protein is administered to a plant having or at risk of having an infection by a pathogen. Application of a protein to a plant may be by foliar application, such as spraying, brushing, or any other method. The application can be to the entire plant or to a portion thereof, such as a leaf, a flower, a fruit, a seed, or a vegetable. The composition may be aqueous or non-aqueous. In one embodiment, the composition includes agents to aid in the ability of the protein to remain associated with the surface of the plant. The composition may include other agents to aid in the topical application of a protein including, but not limited to, a surfactant (for instance, anionic, cationic, amphoteric, nonionic), a biosurfactant, a wetting agent, a penetrant, a thickener, an emusifier, a spreader, a sticker, an oil, an alkyl polyglucoside, an organosilicate, an inorganic salts, or a combination thereof. The composition administered can include an antimicrobial, antiviral, and/or antifungal agent. Optionally, the treatment can include separate administration of an antimicrobial, antiviral, and/or antifungal agent.

Also provided by the present disclosure are plants that include a protein described herein. The protein can be present on the entire plant or on a part of the plant. Also provided herein are methods for applying a protein to a plant, or a part of the plant, with a composition that includes a protein described herein.

Rot Prevention

In another embodiment, a composition can be used to prevent or reduce rot, e.g., prevent or reduce food spoilage. The method includes contacting a product susceptible to rot, such as fruit, fresh produce, fish, meat, or a dairy product, an effective amount of a composition that includes a protein described herein. Examples of produce that can be treated includes potato, sweet potato, tomato, carrots. etc. Examples of microbes with rot functions controlled by quorum sensing and causing rot in produce include, but are not limited to, Pseudomonas sp., Pectobacterium spp., Pectobacterium carotovorum ssp. Atrosepticum, Pectobacterium carotovorum ssp. Carotovorum, Pectobacterium chrysanthemi.

Changing Populations

In one embodiment, a method is for altering a community of microbes. The inventors have determined that inhibition of quorum sensing can result in a change in the composition of a community of microbes. In one embodiment, the community includes a biofilm. In one embodiment, the community is planktonic. An example of a community is a microbiome, such as a microbiome present in the gastrointestinal tract. Changing the community can include altering the relative abundance of one or more different strains, altering the presence or absence of one or more different strains, or a combination thereof. The use of a lactonase can induce a dramatic composition change in a microbial community. This finding was unexpected because lactonases were previously described to solely affect AHL-utilizing microbes, but not microbial communities that include bacteria not using AHLs.

The population that is changed in a community can be one or more microbes that exhibit quorum sensing, one or more microbes that do not exhibit quorum sensing, or a combination thereof. In one embodiment, the population of at least 1, 2, 3, 4, 5, 6, 7, 8, 9, 10, 15, 20, 30, 40, 50, 60, 70, 80, 90, 100 or more different microbes in a community, e.g., a biofilm, can be changed. The change can be evaluated at the level of genus or species. In one embodiment, the alteration can be an increase or a decrease in the relative abundance of a microbe. As used herein, “relative abundance” refers to the amount of a microbe relative to other microbes in a community. For example, the relative abundance can be determined by generally measuring the presence of a particular microbe compared to the total presence of microbes in a sample. The change in the relative abundance can be measured, for instance, as colony forming units or by evaluating genomic DNA using next-generation sequencing methods. The relative abundance of a microbe can be increased or decreased by at least 10%, at least 20%, at least 30%, at least 40%, at least 50%, at least 60%, at least 70%, at least 80%, at least 90% or 100% compared to the relative abundance before the biofilm is exposed to a protein described herein. In this aspect of the disclosure, an “effective amount” is an amount effective to change a community of microbes, for instance, a biofilm.

The location of the community with the population that is to be changed is not intended to be limiting. For instance, in one embodiment a community can be associated with an infection and can be in any location described herein related to an infection. For instance, in one embodiment a community can be associated with a condition and can be in any location described herein related to a condition. In one embodiment a community, e.g., a biofilm, can be associated with a surface. In one embodiment, the method can be used to alter the population of a microbiota of an individual. Examples of microbiota include, but are not limited to, gut microbiota and skin microbiota.

In one embodiment, a method is for counteracting intestinal microbiota dysbiosis. The method includes administering to an animal an effective amount of composition that includes a protein described herein. Intestinal microbiota dysbiosis is a condition related with the pathogenesis of intestinal illnesses (irritable bowel syndrome, celiac disease, and inflammatory bowel disease) and extra-intestinal illnesses (obesity, metabolic disorder, cardiovascular syndrome, allergy, and asthma) (Gagliardi et al., Int J Environ Res Public Health, 2018, 15(8): 1679). In one embodiment, the administration of a composition described herein results in displacement of potentially pathogenic bacteria and a rebalance of an individual's microbial community to a eubiotic state. In one embodiment, signs related to a condition associated with intestinal microbiota dysbiosis such as, but not limited to, irritable bowel syndrome, celiac disease, inflammatory bowel disease, obesity, metabolic disorder, cardiovascular syndrome, allergy, or asthma, are reduced or completely removed.

Kits

Also provided are kits. A kit can be for any use described herein, including but not limited to treating a biofilm, disinfecting a surface, treating an infection, reducing spoilage, or changing a population.

The kit includes at least one of the proteins described herein (e.g., one, at least two, at least three, etc.), a polynucleotide encoding a protein described herein, or a genetically modified microbe described herein. Optionally, other reagents such as buffers and solutions are also included. Instructions for use of the packaged antibody or protein are also typically included. As used herein, the phrase “packaging material” refers to one or more physical structures used to house the contents of the kit. The packaging material is constructed by routine methods, generally to provide a sterile, contaminant-free environment. The packaging material may have a label which indicates that the proteins can be used for one or more of the uses described herein. In addition, the packaging material contains instructions indicating how the materials within the kit are employed to treat a biofilm, disinfect a surface, treat an infection, reduce spoilage, and/or change a population. As used herein, the term “package” refers to a container such as glass, plastic, paper, foil, and the like, capable of holding within fixed limits the proteins, and other reagents. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

### Creation of Improved Mutants of a Quorum Quenching Lactonase from Geobacillus Caldoxylosilyticus

### Structural Studies of a Novel Quorum Quenching Enzyme Reveals Two Distinct, Productive Binding Modes for Lactones

### Abstract

Quorum quenching lactonases are enzymes capable of hydrolyzing N-acyl homoserine lactones (AHLs), molecules known as signals in bacterial communication. This signal disruption by lactonases was previously reported to inhibit behaviors regulated by quorum sensing such as the expression of virulence factors and the formation of biofilms. Here, we report the enzymatic and structural characterization of a novel lactonase isolated from the thermophilic bacteria Geobacillus caldoxylosilyticus, dubbed GcL. The enzymatic characterization revealed that GcL is both a broad spectrum and a highly proficient lactonases, with k_{cat}/K_{M} values in the range of 10⁴ to 10⁶ M⁻¹·s⁻¹. Additionally, and in contrast to most characterized lactonases, GcL exhibits low K_{M} values (0.5-20 μM). Crystal structures of GcL bound to HEPES and to the substrate C6-AHL suggests that these low K_{M} values are due to the presence of a hydrophobic patch that participates in the accommodation of the aliphatic acyl chains of AHLs. In addition to the structure bound to C6-AHL, we solved a structure bound to ε-caprolactone. Unexpectedly, while both of these substrate molecules are hydrolyzed with high rates by GcL, they bind on the bi-metallic nuclear center with opposite orientations. Interestingly, both binding modes are compatible with a nucleophilic attack of the putatively catalytic metal-bridging water molecule. These structures highlight the high level of plasticity of GcL's active site, possibly accounting for its broad activity spectrum, including its promiscuous phosphotriesterase activity. The high catalytic versatility of GcL makes it an excellent candidate for engineering studies aiming at improving its current lactonase activities or evolving new functions.

### INTRODUCTION

Quorum sensing (QS) is a communication system used by numerous microorganisms to coordinate various behaviors. QS is based on small molecules secreted by microorganisms, such as N-acyl-L-homoserine lactone (AHLs)¹. Once a concentration threshold of signal molecules is reached, a certain concentration of microorganisms is reached, then AHLs bind to a receptor and thereby regulate the expression of gene patterns, including genes involved in virulence, biofilm production and others². Many enzymes, named Quorum Quenching enzymes (QQ), are known to degrade the QS signals³ and represent promising tools in numerous fields, including in therapeutics, in the prevention of marine biofouling and in plant protection⁴.

AHLs-degrading enzymes, such as lactonases, have been isolated from fungi, mammals, archea, plants and bacteria⁵. Lactonase enzymes belongs to three main families: the Phophotriesterases-like lactonases (PLLs)⁶ are characterized by an (α/β)₈ fold and found in archea and bacteria. The second family, the paraoxonases⁷ were isolated from mammals exhibit a six bladed β-propeller fold.

The third lactonase family is the Metallo-β-lactamase-like lactonases (MLLs)⁵, and is exemplified by the first isolated and studied representative, AiiA from Bacillus thuringiensis⁸. The MLLs exhibit a conserved dinuclear metal binding motif, HXHXDH, involved in the binding of two metal cations and possesses an αβ/βα fold⁹. Numerous MLLs were isolated and characterized but only few were studied structurally. In fact, only the structures of AiiA¹⁰⁻¹³, AiiB from Agrobacterium tumefaciens¹⁴, AidC from Chrysseobacterium sp. Strain StRB1261¹⁵ and AaL from Alicyclobacter acidoterrestris¹⁶ were resolved. The active site of MLLs is composed of a bi-metallic nuclear center bridged by a putative catalytic water molecule that is hypothesized to attack the electrophilic carbon atom of the lactone ring⁹. Nevertheless, questions surrounding acid catalysis to help the departure of the leaving alcoholate group, as well as the structural determinants for the substrate specificity of these enzymes are still unclear.

GcL is an enzyme isolated from the thermophilic bacteria Geobacillus caldoxylosilyticus. GcL is one of the rare thermophilic MLLs representative, with a half-life at 75° C. of 152.5±10 min¹⁷. This protein present 28% sequence identity with the most known MLLs enzyme, AiiA, and the closest characterized enzyme is AaL (85% sequence identity) (FIG. 1). GcL possesses a broad activity spectrum against AHLs and high catalytic efficiency (K_{cat}/K_{M}=10⁴-10⁶ s⁻¹ M⁻¹)¹⁷. Here we report a comprehensive enzymological and structural study of GcL. We show that GcL is a proficient lactonase against both AHLs and aliphatic γ- and δ-lactones. The obtained crystal structures of GcL solved with HEPES, C6-AHL as well as ε-caprolactone reveals the presence of the hydrophobic patch to accommodate acyl chains of substrates, as well as differences in the binding of the lactone rings of AHLs and lipophilic lactones. This very first structural data of a lactonase bound to two different type of lactones will serve as foundation to future mechanistic and engineering studies on these enzymes.

### Methods

Sequence blast. The FASTA sequence of the first structurally characterized MLLs enzyme, AiiA from the organism Bacillus thuringiensis¹¹, was blasted against the non-redundant protein sequences database. Due to their higher compatibility with biotechnological applications, we selected enzymes from thermophiles. Thereby, the protein GcL (WP_017434252.1) isolated from the thermophilic organism Geobacillus caldoxylosilyticus was selected.

Protein production and purification. The protein was produced in Escherichia coli strain BL21(DE3)-pGro7/GroEL strain (TaKaRa). A strep tag (WSHPQFEK (SEQ ID NO:8)) has been added to the sequence along with a TEV sequence (ENLYFQS (SEQ ID NO:9)). The protein was produced at 37° C. in 2 liters of the autoinduceur media ZYP (100 mg·ml-1 ampicillin and 34 mg·ml⁻¹ chloramphenicol). When OD_{600 nm} reached the exponential growth phase, the culture was induced with 2 mM CoCl2 and 0.2% of L-arabinose. The induction process temperature was at 18° C. overnight. Cells were harvested by centrifugation and the pelleted cells were resuspended in lysis buffer (150 mM NaCl, 50 mM HEPES pH 8.0, 0.2 mM CoCl2, 0.1 mM PMSF and 25 mg·ml lysozyme) and left in ice during 30 minutes. Then, cells were sonicated in 3 steps during 30 seconds (1 pulse-on; 2 pulse-off) at amplitude 45 (Q700 Sonicator, Qsonica, USA). After sonication, the supernatant lysate were loaded on a Strep Trap HP chromatography column (GE Healthcare) in PTE buffer consisting of 50 mM HEPES pH 8.0, 150 mM NaCl and 0.2 mM CoCl₂ at room temperature. The Strep Tag was cleaved by using the Tobacco Etch Virus protease (TEV, reaction 1/20, w/w) during 20 hours at 4° C. At last, the sample was concentrated been loaded on a size exclusion column (Superdex 75 16/60, GE Healthcare) to obtain a pure protein. The protein identity and purity were controlled by Coomassie-stained SDS-PAGE. The fractions containing the pure protein were blend and concentrated to 11.66 mg·ml⁻¹ using a centrifugation device (Vivaspin 15R, Sartorius, Germany).

Kinetic measurements. The determination of GcL catalytic efficiency was performed by using a microplate reader (Synergy HTX, BioTek, USA) and the software Gen5.1 over a range of substrates (FIG. 2). The reactions were operate in a 96-well plate at a path length of 5.8 mm for a 200 μl reaction volume at room temperature. The catalytic parameters were achieved by fitting the data to Michaelis-Menten equation with the Graph-Pad Prism 5 software. When, Vₘₐₓ was not reached, the catalytic efficiency was determined by fitting the linear part of Michaelis-Menten plot to a linear regression using Graph-Pad Prism 5.

Lactonase assay. The AHL lactonolysis consisting in the opening of the lactone ring generates a proton, and leads to an acidification of the media. This property allowed the characterization of the lactonases by using a pH indicator assay. To perform the experiment 5 μl of enzyme were added to a solution containing 10 μl of substrates at various concentrations and 185 μl of lactonase buffer (2.5 mM Bicine pH 8.3, 150 mM NaCl, 0.2 mM CoCl2, 0.2 mM cresol purple, 0.5% DMSO). This assay was performed at 25° C. and the time course of the lactones hydrolysis was recorded at 577 nm. The characterization of the enzyme was proceeding against a large panel of AHLs: C4-AHL, C6-AHL, C8-AHL, C10-AHL, 3-oxo-C8-AHL and against γ-Butyrolactone, γ-Heptalactone, γ-Nonalactone, γ-Decanolactone, δ-Valerolactone, δ-Octanolactone, δ-Nonalactone, δ-Decalactone, ε-Caprolactone, ε-Decalactone, Whiskey lactone.

Paraoxon assay. The determination of the activity against the organophosphate paraoxon-ethyl was performed through a colorimetric assay. In fact, the paraoxon-ethyl hydrolysis generates p-nitrophenolate anion which is colored yellow. The assay was performed by measuring the time course hydrolysis (ε_{405 nm}=17 000 M⁻¹ cm⁻¹) of paraoxon-ethyl in the activity buffer (50 mM HEPES pH 8.0, 150 mM NaCl, 0.2 mM COCl₂).

Crystallization. Crystallization was performed with protein sample concentrated at 11.5 mg·mL by using the sitting-drop vapor-diffusion method. The initial screening was operate at 292 K in a 96-well plate with the commercial kit JCSG+ at different protein: precipitant ratios (1:1, 1:2 and 1:3). The condition at 1.25 M ammonium sulfate and 0.1 M sodium acetate pH 5.5 produced the best crystals. A refinement of crystallization conditions was accomplished to improve the crystals quality by varying at 1 to 2.25 M of ammonium sulfate concentrations (from 1 to 2.25 M) and pH (pH 4.0 to 5.5; 0.1 M sodium buffer). Good diffraction quality crystals appeared after 1 day at 292 K. Before diffraction, the crystals were cryoprotected in a solution composed of 30% PEG 400 and frozen in liquid nitrogen. Structures in complex were obtained by soaking during 5 minutes the crystals in a solution containing the cryoprotectant and 20 mM of lactone substrates.

Data collection, structure resolution and refinement. X-ray diffraction datasets were collected at 100 K using synchrotron radiation on the 23-ID-B beamline at the Advanced Photon Source (APS, Argonne, Ill., USA) using a MAR CCD detector for the structure bound to an HEPES molecule, an Eiger for the structure complexed with C6-AHL and ε-caprolactone. Diffraction data were collected at a wavelength of 1.033 \acute{Å}, and depending of the data set between 400 and 1100 images were collected, with 0.2° or 0.5 oscillation steps and an exposure time of 0.2 s. The structures were resolved in C2 space group for the HEPES and C6-AHL bound but their unit cells parameters differs probably due to substrate binding. The structure containing the ε-caprolactone was resolved in a R3.

The integration and the scaling of the X-ray diffraction data were performed using the XDS package¹⁸. The molecular replacement was performed using AiiB structure as a model (PDB: 2R2D)¹⁴ (44% sequence identity) and using MOLREP¹⁹. Then, an automated model reconstruction of GcL was done using Buccaneer²⁰ before to be manually improved with Coot program²¹. Cycles of refinement were performed using REFMAC²². Statistics are shown in Table 2.

TABLE 2
Data collection and refinement statistics of GcL structures

| | | |
|---|---|---|
| HEPES | C6-AHL | c-caprolactone |
| bound structure | bound structure | Bound structure |

DATA COLLECTION

| Diffraction source | APS Argonne 231D-B | | APS Argonne 231D-D | | APS Argonne 231D-D | |
|---|---|---|---|---|---|---|
| Wavelength (Å) | 1.03323 | | 1.033199 | | 1.033200 | |
| Detector | MAR CCD | | EIGER | | EIGER | |
| Rotation range per image (°) | 0.5 | | 0.2 | | 0.2 | |
| Total rotation range (°) | 200 | | 220 | | 166 | |
| Space group | C2 | | C2 | | R3 | |
| Unit-cell parameters (Å) | a = 145.42, b = 108.68, | | a = 160.89, b = 108.84, | | a = 108.25 , b = 108.25, | |
| | c = 78.74; α = γ = 90.000, | | c =97.36; α= γ = 90.000, | | c = 223.58; α = β = 116.937, | |
| | β = 115.845 | | β = 116.937 | | γ = 120.000 | |
| Resolution range (Å) | 1.6 | (1.7-1.6) | 2.1 | (2.2-2.1) | 2.15 | (2.25-2.15) |
| N° of reflections (last bin) | 602221 | (99530) | 357178 | (47597) | 254968 | (33441) |
| N° of unique reflections (last bin) | 144425 | (23974) | 86119 | (11216) | 53150 | (6795) |
| Completeness (%) (last bin) | 99.6 | (99.7) | 98.6 | (99.2) | 100.0 | (100.0) |
| Redundancy | 4.17 | (4.15) | 4.15 | (4.23) | 4.79 | (4.92) |
| <I/σ(I)> | 26.50 | (3.1) | 11.07 | (3.09) | 7.15 | (2.18) |
| Rₘₑₐₛ (%) | 3.3 | (64.5) | 8.5 | (57.9) | 13.7 | (72.5) |

REFINEMENT STATISTICS

| | | | |
|---|---|---|---|
| Rfree/Rwork | 15.17/ 19.73 | 19.79 /22.63 | 20.61/ 23.36 |
| N° of total model atoms | 7065 | 6942 | 4514 |
| Ramachandran favored (%) | 96.46 | 95.27 | 96.47 |
| Ramachandran outliers (%) | 0.00 | 0.12 | 0.00 |
| Generously allowed rotamers (%) | 2.14 | 2.24 | 2.23 |
| Rmsd from ideal | | | |
| Bond lenghts (Å) | 0.0258 | 0.019 | 0.016 |
| Bond angles (°) | 2.3575 | 1.973 | 1.772 |

Anomalous X-ray scattering data. The chemical composition of the metals located in GcL active site has been determined through two anomalous X-ray data collections. Because we used CoCl₂ during the induction step, and because lactonases were previously reported to bind cobalt^{6, 23, 24}, we collected two sets of data at higher (7,859 KeV) and lower (7,715 KeV) energy than the Co-K edge. Data collection statistics are shown in Table 3.

TABLE 3
Anomalous data collection statistics at a higher and lower
energy than Co-edge.
DATA COLLECTION

| | Higher energy than Co-Edge | | Lower energy than Co-Edge | |
|---|---|---|---|---|
| Diffraction source | APS Argonne 231D-D | | APS Argonne 231D-D | |
| Wavelength (Å) | 1.607053 | | 1.577603 | |
| Detector | EIGER | | EIGER | |
| Rotation range per image (°) | 0.5 | | 0.5 | |
| Total rotation range (°) | 360 | | 360 | |
| Space group | C2 | | C2 | |
| Unit-cell parameters (Å) | a = 145.95, b = 107.92, | | a = 145.06, b = 108.56, | |
| | c = 78.97; α = γ = 90.000, | | c = 78.87; | |
| | β = 115.906 | | α = γ = 90.000, β = 115.869 | |
| Resolution range (Å) | 2.65 | (2.75-2.65) | 2.6 | (2.7-2.6) |
| N° of reflections (last bin) | 212499 | (22648) | 222419 | (24187) |
| N° of unique reflections (last bin) | 61761 | (6483) | 65488 | (6979) |
| Completeness (%) (last bin) | 97.7 | (97.2) | 97.4 | (97.2) |
| Redundancy | 3.44 | (3.49) | 3.39 | (3.46) |
| <I/σ(I)> | 7.42 | (2.22) | 12.63 | (2.68) |
| Rₘₑₐₛ (%) | 12.2 | (66.9) | 7.2 | (52.8) |

### Results and Discussion

### GcL is a Highly Proficient, Broad Spectrum Lactonase

The catalytic parameters of GcL were evaluated for a broad range of lactones including AHLs, 3-oxo-AHLs, γ-lactones, ε-lactones, δ-lactones and the whiskey lactone (Table 4). We show that GcL is highly active against AHLs with both short and long acyl chains, exhibiting catalytic efficiencies ranging between 10⁴ to 10⁶ M⁻¹·s⁻¹. Moreover, GcL is also highly active against γ-, δ-, ε- and whiskey-lactone with k_{cat}/K_{M} ranging between 10⁵ to 10⁶ M⁻¹·s⁻¹. The slowest tested substrate is C4-AHL, with a catalytic efficiency of 8.3 (2.2)×10⁴ M⁻¹ s⁻¹, while the best substrate is 3-oxo-C8-AHL (k_{cat}/K_{M}=4.3 (±0.7)×10⁶ M⁻¹ s⁻¹). Kinetic data revels that GcL exhibits unusually low K_{M} values for a large majority of the tested substrates (0.67-21.1 μM, with the exception of C4-AHL), as compared to other known lactonases. This contrasts with AiiA, AiiB (K_{M}˜1600-5600 μM^{13,14}) and other classes of lactonases (e. g. PLLs and PONs; K_{M} ˜ 50-500 μM^{11, 23, 25-27}). These high K_{M} values for most known lactonases contrasts with concentration thresholds for quorum sensing activation that are in the range of ˜5 nM^{4, 28, 29}. Therefore, GcL may be a promising enzyme candidate for potent quorum quenching.

TABLE 4
Enzymatic characterization of GcL enzyme

| Substrates | K_{cat} (s⁻¹) | k_{M} (μM) | K_{cat}/k_{M} (s⁻¹ M⁻¹) |
|---|---|---|---|
| C4-AHL (1)* | 19.06 ± 1.51 | 229 ± 57 | (8.3 ± 2.2) × 10⁴ |
| C6-AHL (1)* | 8.95 ± 0.48 | 7.97 ± 1.89 | (1.1 ± 0.3) × 10⁶ |
| C8-AHL (1) | 1.29 ± 0.04 | 3.12 ± 0.75 | (4.1 ± 1.0) × 10⁵ |
| C10-AHL (l)* | 5.48 ± 0.37 | 1.45 ± 0.47 | (3.8 ± 1.3) × 10⁶ |
| 3-oxo-C8-AHL (l)* | 9.48 ± 0.35 | 2.19 ± 0.37 | (4.3 ± 0.8) × 10⁶ |
| γ-Butyrolactone | 2.49 ± 0.15 | 21.1 ± 7.60 | (1.2 ± 0.4) × 10⁵ |
| γ-Heptalactone | 1.77 ± 0.06 | 2.01 ± 0.71 | (8.8 ÷ 3.1) × 10⁵ |
| γ-Nonalactone | 18.14 ± 0.9 | 11.6 ± 3.10 | (1.6 ± 0.4) × 10⁶ |
| γ-Decanolactone | 2.69 ± 0.11 | 7.14 ± 1.03 | (3.8 ± 0.6) × 10⁵ |
| δ-Valerolactone | 1.27 ± 0.03 | 5.67 ± 0.94 | (2.2 ± 0.4) × 10⁵ |
| δ-Octanolactone | 12.58 ± 0.85 | 18.3 ± 7.00 | (6.9 ± 2.6) × 10⁵ |
| δ-Nonalactone | 2.01 ± 0.14 | 8.05 ± 2.5 | (2.5 ± 0.8) × 10⁵ |
| δ-Decalactone | 4.1 ± 0.18 | 3.13 ± 0.9 | (1.3 ± 0.4) × 10⁶ |
| ϵ-Caprolactone | 8.79 ± 0.43 | 10.6 ± 3.20 | (8.3 ± 2.5) × 10⁵ |
| ϵ-Decalactone | 1.01 ± 0.04 | 1.05 ± 0.38 | (9.6 ± 3.5) × 10⁵ |
| Whiskey lactone | 9.34 ± 0.39 | 0.67 ± 0.21 | (1.4 ± 0.4) × 10⁵ |
| Paraoxon-ethyl | ND | ND | (3.1 ± 0.2) × 10¹ |

The standard deviation values for each parameters is given.
ND notification is corresponding to kinetics data not fitting for Michaelis-Menten equation due to a too high or too low catalytic rate. Data from¹⁷

Additionally, we tested the ability of GcL to degrade the insecticide derivative paraoxon, and determined that it is capable of degrading it, albeit with slow rate. This promiscuous activity of GcL is consistent with previous observations in other lactonases, primarily from the PLL family, such as VmoLac²³ and SsoPox³⁰ which exhibit higher phosphotriesterase activities. The promiscuous ability of lactonases to degrade the phosphotriester paraoxon suggest an evolutionary link between lactonases and phosphotriesterase^{31, 32} In fact, lactonases has been proposed as progenitors of the insecticide-degrading enzyme PTE³², which emerged during the last 70 years to degrade synthetic insecticides, the organophosphates. Therefore, the ability of GcL to degrade paraoxon is consistent with previously observed catalytic linkage between lactonases and PTEs³¹⁻³³.

### Structural Analysis

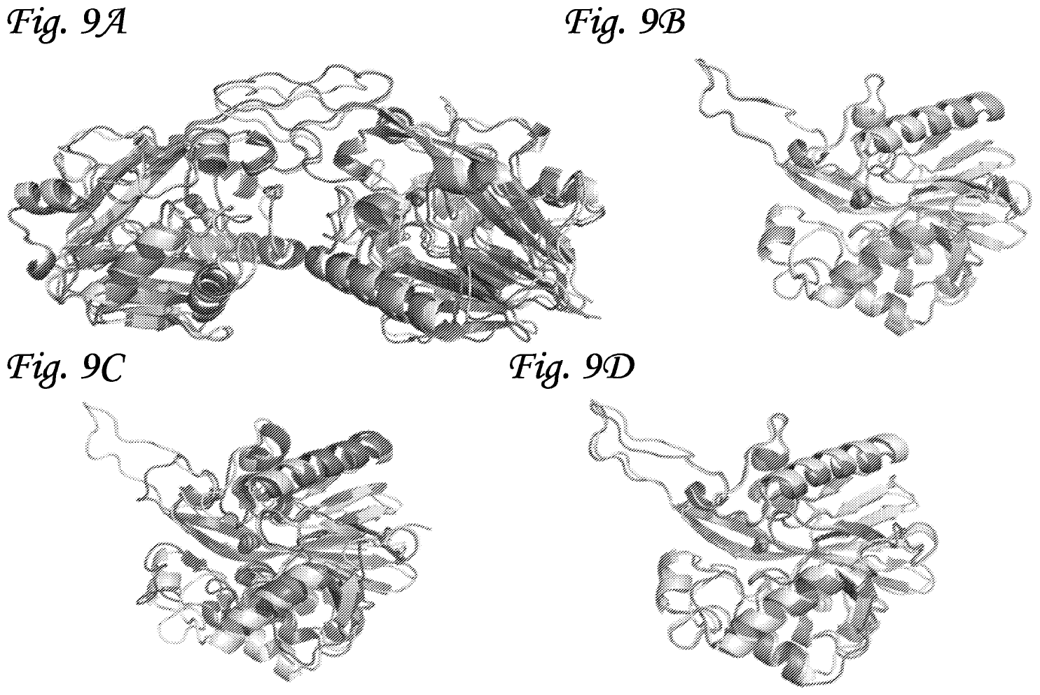

Crystal structure of GcL. The crystal structure of GcL was solved by using structure of AiiB (PDB: 2R2D) from Agrobacterium tumefaciens (44% sequence identity) as a search model in a molecular replacement approach. Since then, we solved the structure of the more closely related AaL (from Alicyclobacillus acidoterrestris; 85% sequence identity (PDB: 6CGY)). GcL structure was solved at 1.6 Å in C2 space group and with unit cell parameters of a=145.42, b=108.68, c=78.74, β=115.845 containing 3 monomers (Table 2). The monomer of GcL is roughly globular with overall dimension of 58×40×44 Å, and shows a long protruding loop. This loop is involved in homodimerization (FIG. 9A). The dimer shows overall dimensions of approximately 85×40×44 Å. As expected, GcL exhibits a αβ/βα sandwich fold, typical to the metallo-β-lactamase superfamily, and is similar to that of others MLLs (AiiA^{10, 11}, AiiB¹⁴, AidC¹⁵ and AaL¹⁶). The active site of GcL is occupied by an HEPES molecule.

The overall structure of GcL is very similar to AaL with a root mean square deviation (r.m.s.d) of 0.42 Å (over 275 α-carbon atoms)(FIG. 9B) and to AiiB (0.89 \acute{Å}over 273 α-carbon atoms) (FIGS. 9A and 9D). However, the structural differences are much more important between GcL and AiiA with an r.m.s.d of 1.22 \acute{Å}(over 180 α-carbon atoms), including the noticeable absence of the external loop 1 in AiiA (FIG. 9C). The finding that GcL, isolated from a thermophilic bacteria, and contrary to AiiA^{10,11}, is organized as a homodimer, is consistent with previous work on thermophilic proteins, highlighting a trend for higher levels of oligomerization in these proteins³⁴. The dimer is characterized by a strong interaction of the protruding loop (A34 to Q42) from both monomers. The dimer interface involves in each monomer 32 residues. The interface is mostly hydrophobic, and engages 12 hydrogen bonds. The interface surface between dimer is 1178.9 Å², a similar value to other dimeric MLL structures such as AiiB and AidC, 1089.1 Å² and 1015.4 Å², respectively.

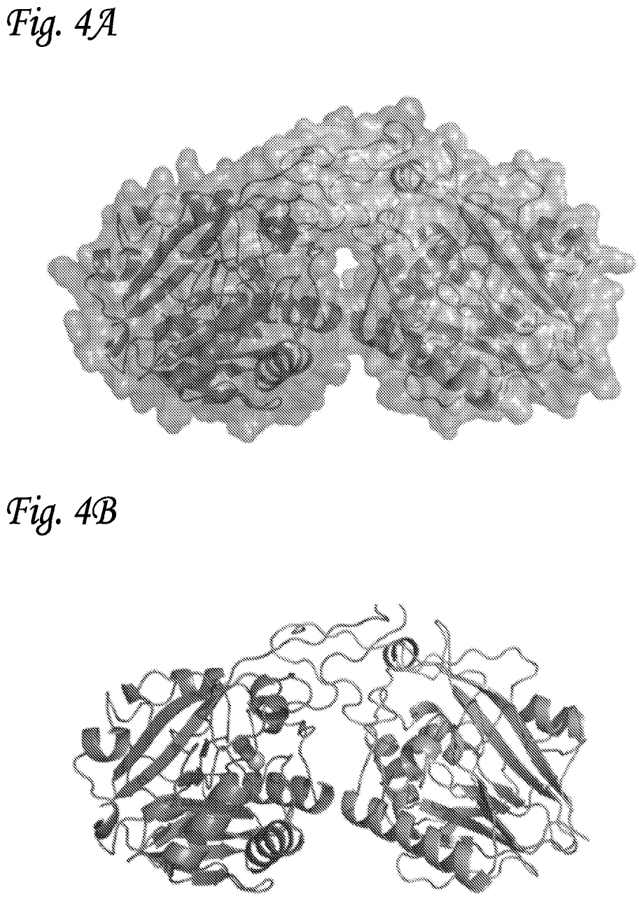

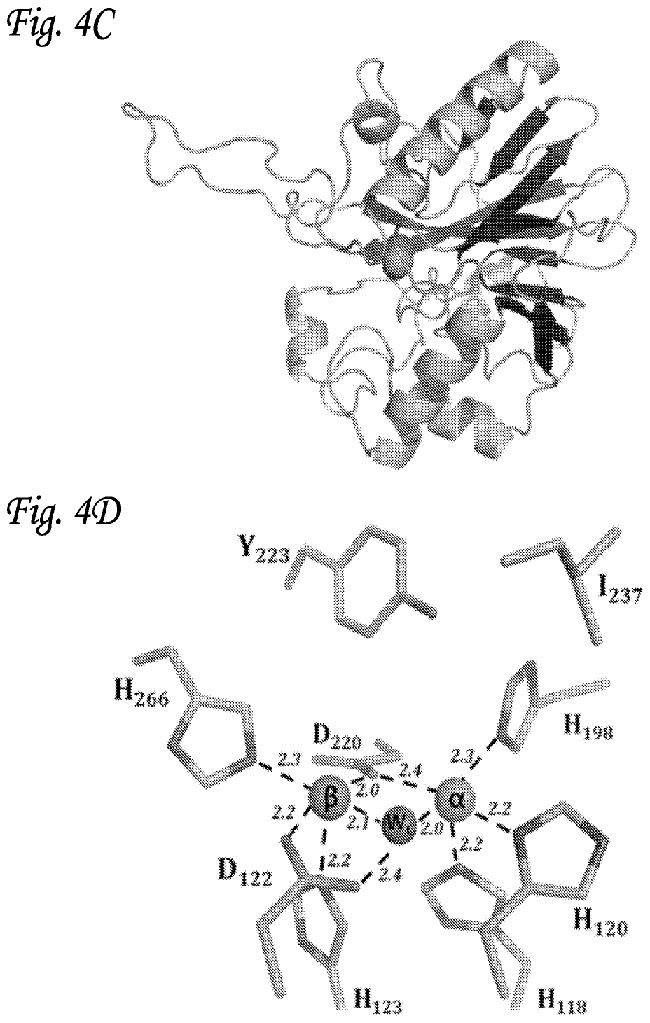

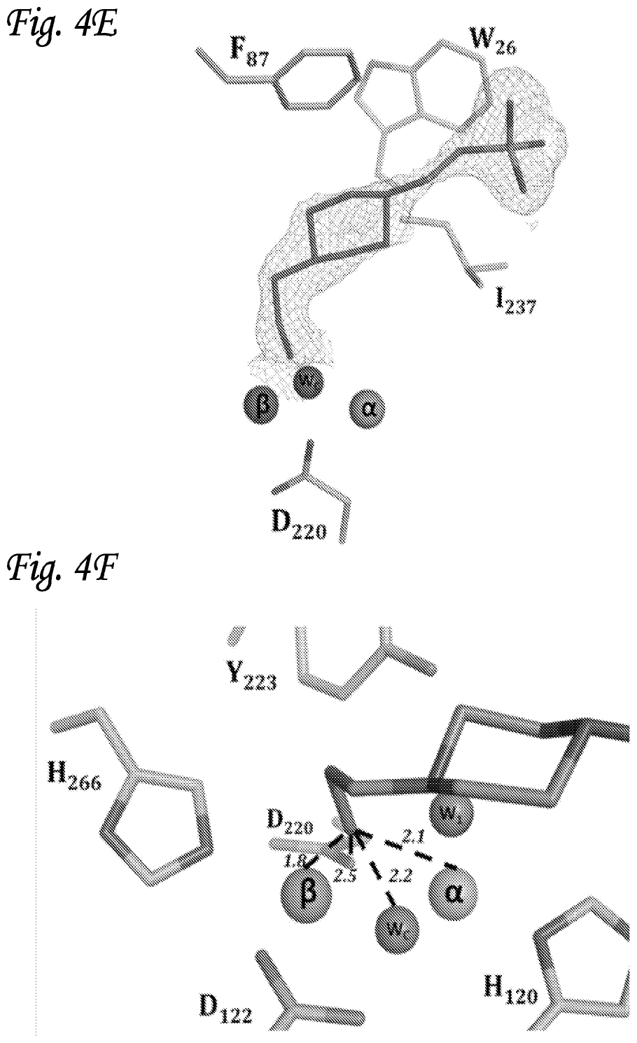

Active site of GcL. GcL active site (FIGS. 4D and 4F) is organized around 2 metals cations coordinated by five histidine residues (118, 120, 123, 198 and 266) and two aspartic acid residues (122 and 220). The α-metal cation is coordinated by H118, H120 and H198 and D220. The β-metal cation is interacting with H123, H266, D122 and D220. The putative catalytic water molecule is bridging the two metals cations. A second water molecule is present in the active site and is bonded to the α-metal cation (FIG. 4F).

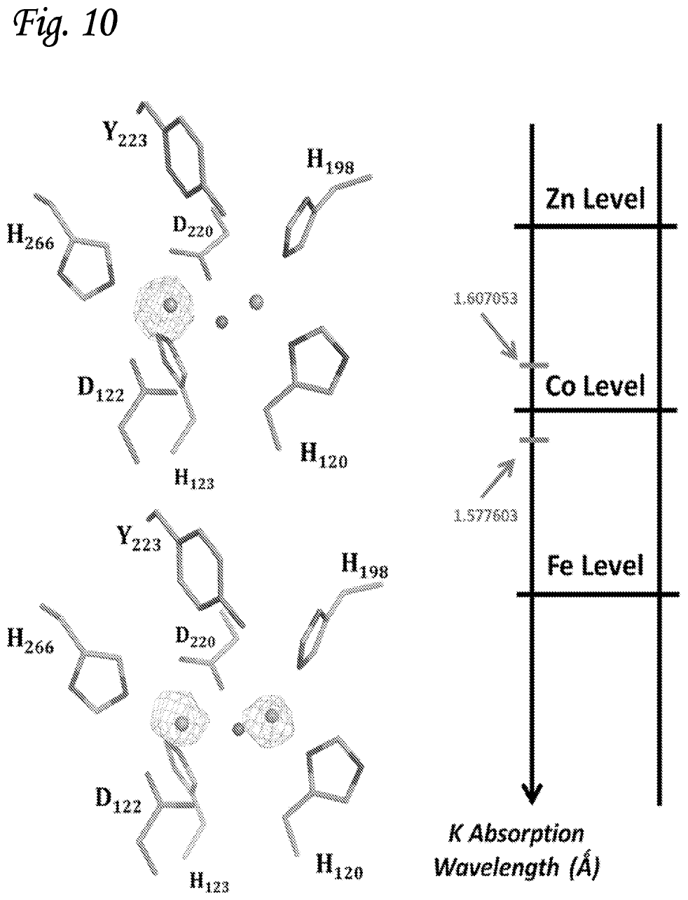

The chemical nature of the metals was investigated using X-ray anomalous data collection around the Co-K edge. Two anomalous data sets at 2.6 and 2.65 \acute{Å} were collected (Table 3). Anomalous X-ray scattering data collected at a higher energy than the Co-Kedge shows two anomalous peaks, revealing that the active site may be occupied by cobalt cations, but not by other common metal cations identified in similar enzymes such as zinc (Zn—K edge is 9.6586 KeV) or nickel (Ni—K edge is 8.3328) (FIG. 10). This result contrasts with some known enzymes from the MLL family that were described to possess two zinc cations in their active site¹²⁻¹⁵ The second dataset at a lower energy than the Co-Kedge reveals only one peak: this unambiguously determines the metal α as a cobalt cation (FIG. 10). The second peak may correspond to other metals, such as iron or manganese. A hetero binuclear iron/cobalt active site was previously unambiguously observed in the lactonase SsoPox, for which the presence of an iron cation was associated to the lower pKₐ of the Fe/H₂O couple as compared to values with other cations²⁴. The presence of a cobalt cation is also compatible with the fact that cobalt cations were added to the protein production steps. Therefore, a heterobinuclear cobalt/iron was modelled in the active site of GcL.

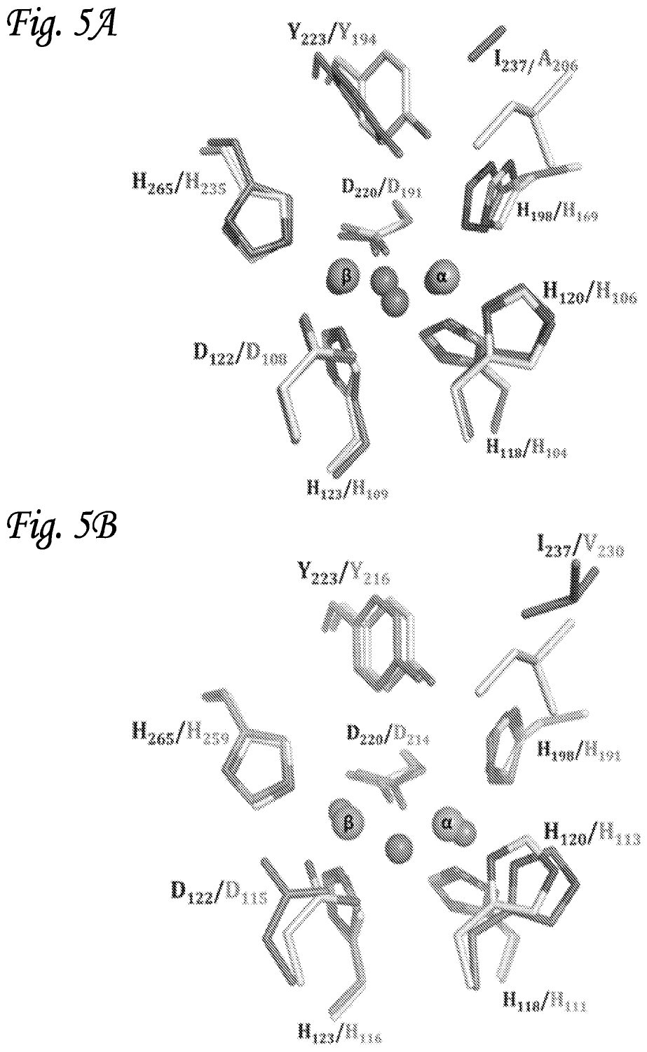

Comparison with other MLLs. GcL active site is overall similar to those of other MLLs (FIGS. 5A and 5B). In fact, GcL presents the same conserve motif HxHxDH than the other MLLs which coordinates the two metals cations of the active site. Nevertheless, several significant differences are visible: indeed, in AiiA structure the tyrosine 223 and the histidine 266 shows different orientations and a shift with the histidine 198 and the aspartic acid 220, as compared to the position of the corresponding residues in GcL (FIG. 5A). Additionally, the distance between the metal cations is larger in AiiB as compared to GcL, yielding to the reorientation of the metal coordinating residues (FIG. 5B). Moreover, the residue 1237, also present in AaL, is replaced by A206 in AiiA and V230 in AiiB (FIGS. 5A and 5B). This amino acid has been proposed to be involved in substrate binding for AaL enzyme.

### Structure of GcL Bound to a HEPES Molecule.

GcL was solved at 1.6 Å bound to a HEPES molecule. HEPES may originate from the buffer used during the protein purification step that contains HEPES. The molecule interact through its alcohol group with the β-metal (1.8 \acute{Å}distance), and not its sulfate moiety (FIG. 5D). In addition to the catalytic, bridging water molecule, a second water molecule is present and liganted to the α-metal cation. The rest of the HEPES molecule fits within the active site hydrophobic crevice.

### Structure of GcL Bound to a C6-AHL Molecule.

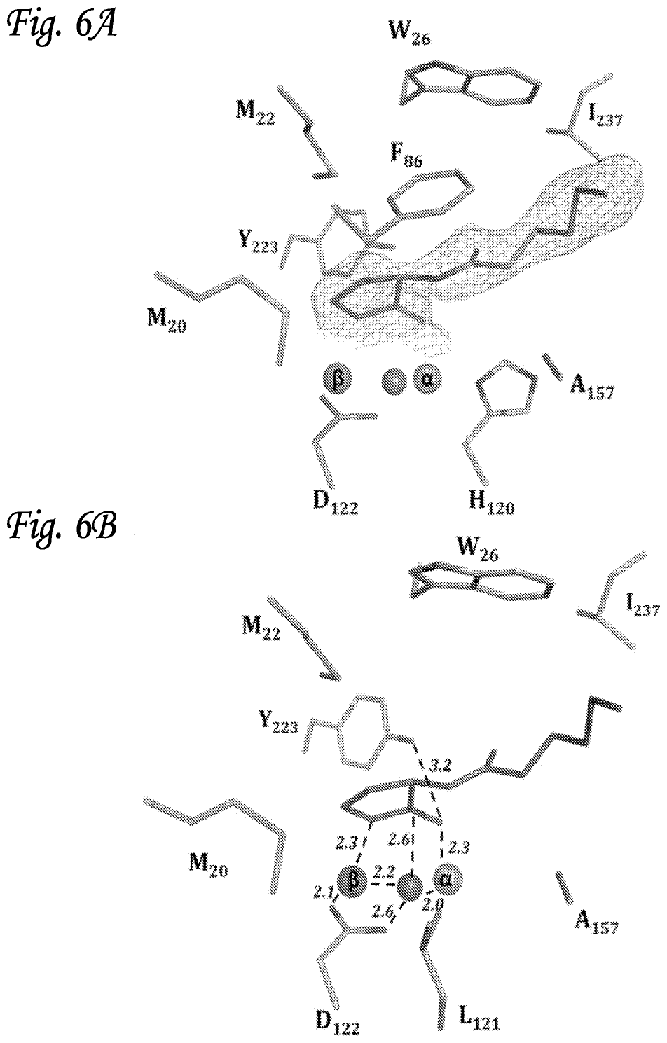

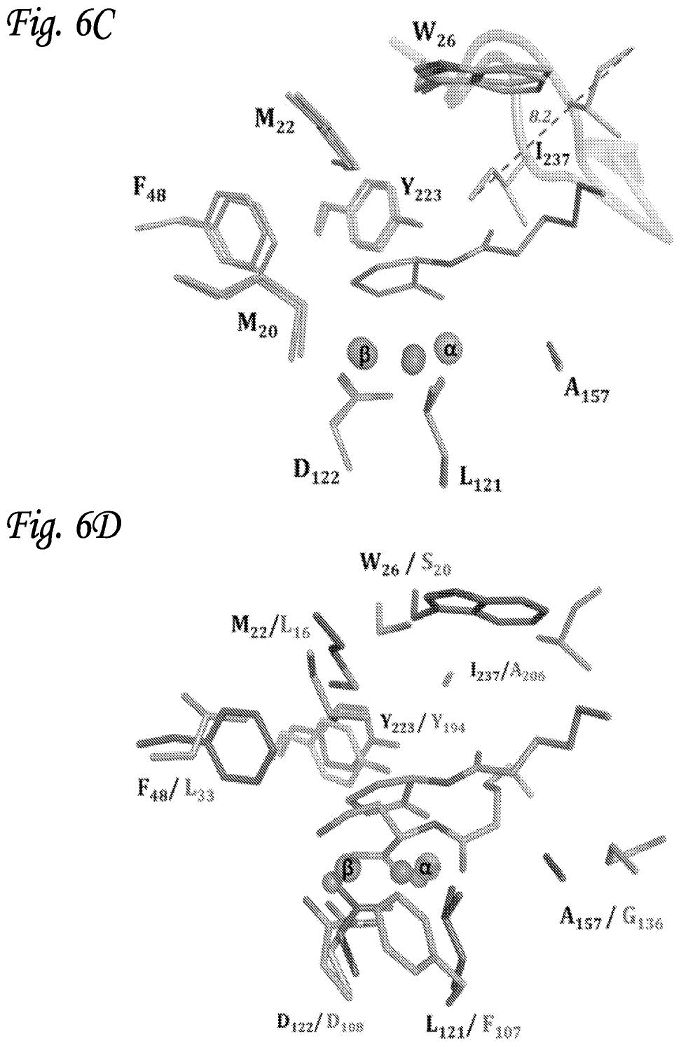

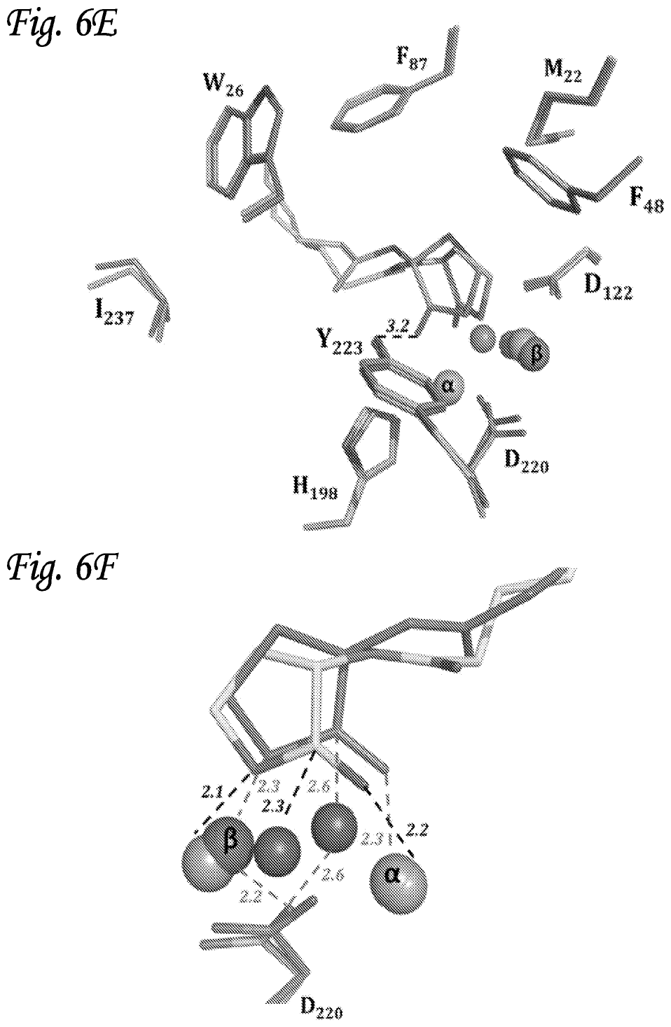

After soaking GcL crystals in the cryoprotectant solution supplemented with 20 mM of C6-AHL for 5 min, the structure of GcL bound to C6-AHL could be solved at 2.1 \acute{Å} (FIG. 6). As a result of soaking, the crystals belong to the same space group than the structure complexed with HEPES (C2), but the unit cell parameters are different (Table 2). The obtained density map unambiguously reveals the presence of the C6 AHL inside the hydrophobic channel of the active site (FIG. 6A). This structure is overall similar to the HEPES bound structure. It is only the second structure of a MLL lactonase bound to a non-hydrolyzed substrate, with the recent work on AaL from our group. Other structures with hydrolytic product were solved for AiiA (PDB: 3DHA, 3DHB, 3DHC, 4J5H^{12,35}.

The lactone ring of the C6 AHL sits on the bi-metallic active site (FIGS. 6A and 6B). The carbonyl oxygen is interacting with the α-metal (2.3 Å) and the hydroxyl group of the tyrosine 223 (3.2 Å). The esteric oxygen of the lactone ring interacts with the β-cobalt (2.3 Å). The two metals cations are bridged by the putative catalytic water (α-metal: 2.0 Å; β-metal: 2.2 Å). The catalytic water is located 2.6 Å away from the electrophilic carbon of the lactone ring, and this binding configuration is compatible with a nucleophilic attack of the bridging water molecule, as previously proposed^{13,24}.

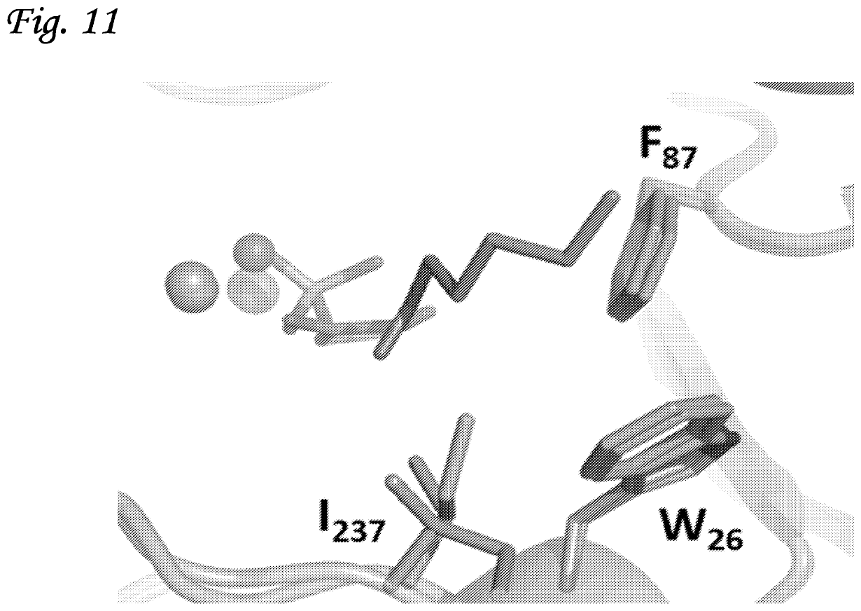

The accommodation of the N-alkyl chain of the AHL is unique: whereas AiiA utilizes a shallow crevice, where longer AHL residues can be stabilized by a phenylalanine clamp³⁵, the binding cleft in GcL is different. Similarly to AaL, the structure shows that the acyl chain interacts with the hydrophobic patch formed by W26, F87 and I237 (FIG. 11) that has no equivalent in other MLLs but in AaL. The presence of a unique hydrophobic patch may contribute to the observed lower K_{M} values of GcL. Additionally, the bound C6-AHL also interacts with two methionine (20, 22), two phenylalanine (48 and 87) a tyrosine (223), a leucine (121), an alanine (157), an tryptophan (26) and an isoleucine (237) (FIGS. 6A-C). Remarkably, as compared to the HEPES-bound structure, the 237-loop adopts a large reorientation upon C6-AHL binding, including a very large reorientation of 1237 side chain (up to 8.2 Å) (FIG. 6C). In presence of HEPES, 1237 points outside of the binding cleft, leading to a larger binding cavity, while it points towards the inside with the bound C6 AHL and interacts with the acyl chain of the substrate. This significant conformational change evidences the putative importance of I237 in the binding of AHLs (FIG. 6D).

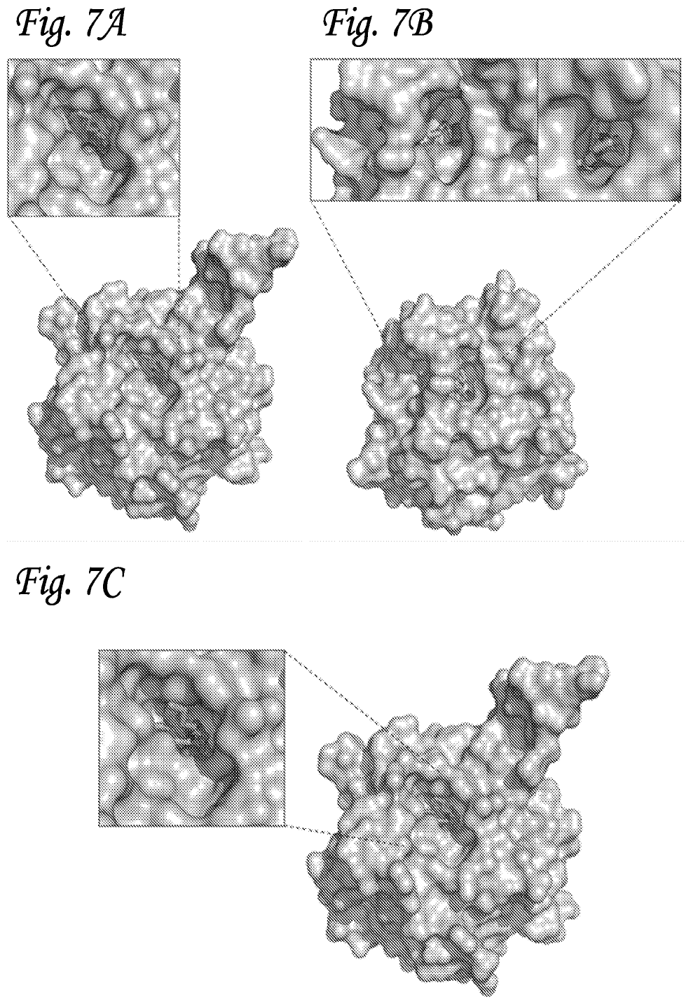

This binding mode is different from the one observed in AiiA with a bound AHL hydrolytic product. Indeed, due to a longer helix in GcL (D140 to R151) as compared to AiiA, GcL possesses only one binding cavity, as opposed to two in AiiA. Interestingly, GcL's active site cavity has an equivalent in AiiA, but is not utilized by the acyl chain of a bound AHL hydrolytic product (FIG. 7C). Therefore, binding of AHLs is likely to be different in both enzymes.

A comparison between the C6-AHL bound structures of GcL and AaL reveals that the binding mode are similar in regards to the acyl chain, with the exception of the positioning of the amide group (FIGS. 6E and 6F). However, significant differences are noticeable in the lactone ring binding mode. First, the metal coordination and the position of the catalytic water molecules are different (FIG. 6F). Distance between metal cations is larger in AaL (4.0 Å) than in GcL (3.5 Å). Secondly, the position of the catalytic water molecule of GcL is nearly equidistant of each metal cations in GcL (2.0 and 2.2 Å), whereas it is much closer of the β-metal cation in AaL (1.6 Å; 2.9 Afrom α-metal). Thirdly, this difference in metal coordination results in residue D220 adopting two distinct conformation in both structure (distant by 0.6 Å). Reasons for these observed changes in the metal cations coordination are not obvious from the structures. It might reside in the different nature of the bound metal cations: AaL binds two cobal cations, while GcL binds one cobalt and possibly an iron cation.

According to the changes in the cations coordination, the binding of the lactone rings to the metal cations is different in both enzymes. Consequently, the distance between the bridging water molecule and the electrophilic carbon atom of the lactone ring is greater in GcL structure (2.6 Å) than in AaL structure (2.3 Å) (FIG. 6F). Even more significant, the hydroxyl group of Y223 interacts with the carbonyl oxygen atom of the lactone ring in the GcL structure (3.2 Å), but not in AaL structure (4.5 Å) (FIG. 6E).

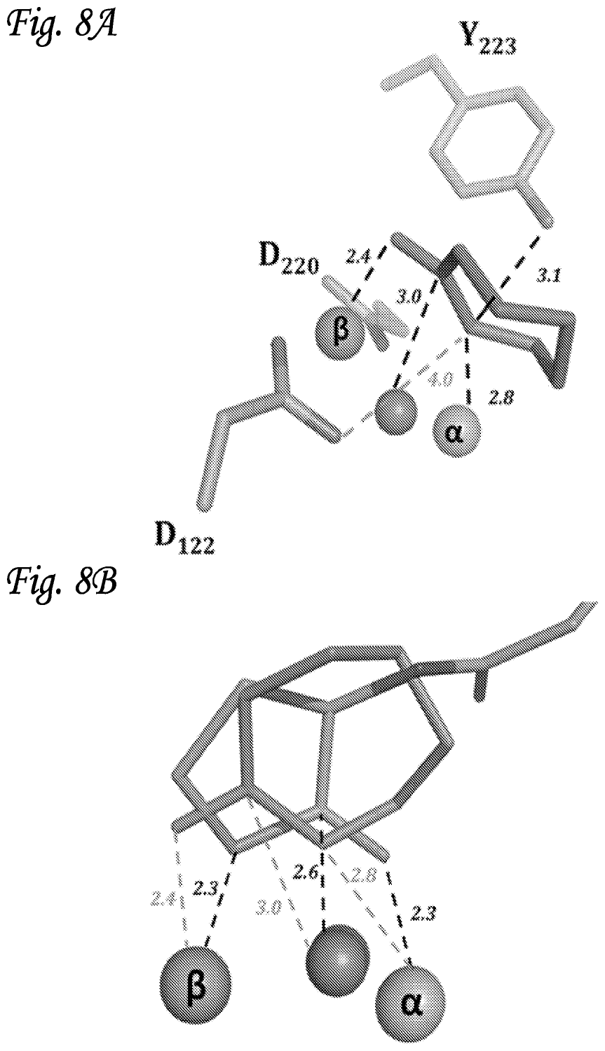

Structure of GcL bound to a β-caprolactone molecule. The structure of GcL bound to ε-caprolactone was solved at 2.15 Å. The 7-atoms lactone ring of the ε-caprolactone sits on the bimetallic active site (FIGS. 8A and 8B). The esteric oxygen atom interacts with the α-cobalt cation (2.8 Å) and Y223 (3.1 Å). The distance of the lactone ring to the α-cobalt cation is significantly longer than for the bound C6-AHL (2.8 Å and 2.3 Å, for C6-AHL and ε-caprolactone bound structures, respectively). The carbonyl oxygen atom of the substrate bind is interacting with the β-iron cation (2.4 Å) and Y223 (3.7 Å). The electrophilic carbon of the lactone ring is sits 3.0 Å away from the catalytic water molecule, and this configuration is compatible with a nucleophilic attack of the bridging water molecule onto the lactone ring.

The comparison of the two GcL structures bound to C6-AHL and to ε-caprolactone shows major differences in their respective substrate binding modes (FIG. 8B). Indeed, the binding orientation of the two lactones ring is inversed. Whereas the β-metal cation interacts with the carbonyl oxygen atom in the C6-AHL bound structure, it interacts with the esteric oxygen atom in the ε-caprolactone-bound structure. Similarly, the α-metal cation interacts with the esteric oxygen atom in the C6-AHL bound structure and with the carbonyl oxygen in the ε-caprolactone-bound structure. Accordingly, the distances between the lactone ring electrophilic carbon atoms and the bridging water molecules are different: it is larger in the ε-caprolactone-bound structure (3.0 Å) than in the C6 AHL-bound structure (2.6 Å). This unexpected change in binding mode may be due the structural differences between these two substrates: the C6-AHL comprise a 5-atom ring, while ε-caprolactone is a 7-atom ring. Additionally, whereas C6-AHL possesses an N-acyl chain that is accommodated by the hydrophobic crevice of the active site, ε-caprolactone has no acyl chain. It is remarkable that both of these molecules are excellent substrates for GcL (k_{cat}/K_{M} is 8.3×10⁵ and 1.1×10⁶ M⁻¹·s⁻¹ for ε-caprolactone and C6-AHL, respectively).

Both of these lactone ring binding modes are compatible with previously proposed catalytic mechanisms, where the metal cation bridging water molecule performs the nucleophilic attack, and the overall distance to the metals and to the water molecule are similar. This remarkable feature evidences the extreme versatility of GcL's active site. This prowess might be the result of selection, as opposed to chance: indeed, some lactonases are unable to degrade both AHLs and oxo-lactones. For example, PLL-B can only degrade oxo-lactones, while PLL-A can degrade both²³.

Regarding the putative acid catalysis, that may be required to protonated the leaving alcoholate, it is interesting to note that the equivalent residue to the previously proposed acid catalyst in AiiA, D122, is distant from the oxygen atoms, including the esteric oxygen atom, of both bound lactones. Instead, Y223 is closer and may play a role in catalysis in GcL. Indeed, Y223 is conserved in all the known MLLs, with the exception of AidC¹⁵ where it is substituted by a His. Interestingly, this residue is also conserved in PLLs^{24,31}, and has been proposed to be implicated in the catalytic mechanism^{10,36}

### Conclusions

The lactonase GcL from the thermophilic bacterium Geobacillus caldoxylosilyticus exhibits a very broad substrate range, being capable of hydrolyzing short and long chain AHLs with high proficiencies. This broad substrate specificity seems common to most of the MLL lactonases identified thus far, including GcL¹⁷, MomL³⁷, AidC³⁸, AaL¹⁶ or AiiA³⁹. Additionally, similarly to AaL, GcL exhibits high catalytic proficiency against δ-lactones and γ-lactones. This is noteworthy, because some γ-lactones are used as QS molecules in Streptomyces and Rhodococcus^{40,41}.

The unusually low K_{M} values of GcL correlates with the presence of a hydrophobic patch in the vicinity of the active site that is unique to GcL structure. Structural analysis of the structure bound to a C6-AHL molecule allows for the identification of the residues interacting with the acyl chain. In particular, a residue within this hydrophobic patch, 1237, adopts largely different conformations (with reorientation of up to 8.2 Å) upon the binding of the C6-AHL molecule, suggesting a potential role in the AHL accommodation. The use of lactonase with low K_{M} values may be of particular interest to increase their quorum quenching abilities. Indeed, the majorities of quorum quenching enzymes identified so far have high apparent dissociation constant values (100-1000 μM). These values contrast with the reported activation threshold of QS for numerous bacteria, in the range of ˜5 nM⁴²⁻⁴⁴. Future investigations will reveal if the use of lactonases with lower K_{M} values result in stronger quenching.

A comparison of the C6-AHL-bound structures of GcL and AaL highlights major difference in the metal cations coordination, as well as in the binding mode of the C6-AHL molecules. In particular, changes in the positioning of the lactone ring on the bi-metallic active sites results in the hydroxyl group of Y223 interacting with the carbonyl oxygen atom of the lactone ring in the GcL structure (3.2 Å), but not in AaL structure (4.5 Å). This feature may account partly for the observed difference in catalytic efficiency of both enzymes for C6-AHL (k_{cat}/K_{M} is 1.7×10^{5 16} and 1.1×10⁶ M⁻¹·s⁻¹ for AaL and GcL, respectively) and might suggest a different role for Y223 in both enzymes.

Additionally, we obtained for the first time structural data for the same lactonase bound to different lactone molecules, namely C6-AHL and ε-caprolactone. Unexpectedly, these two very good substrates of GcL bind onto the bi-metallic active site in opposite orientations. Interestingly, both of these conformations are compatible with a nucleophilic attack by the bridging, putatively catalytic water molecules. This unique finding reveals the extent of the plasticity and the versatility of the active site of GcL, and possibly of other metalloenzymes, as it was observed for the lactonase PONI³³. Such a high catalytic plasticity suggests that lactonases like GcL might exhibit unknown promiscuous catalytic activities (in addition to their phosphotriesterase activity) and constitute prime candidates to evolve new functions.

### CITATIONS FOR EXAMPLE 1

1. Rutherford, S. T. & Bassler, B. L. Bacterial Quorum Sensing: Its Role in Virulence and Possibilities for Its Control. Cold Spring Harb. Perspect. Med. 2, (2012). 2. Dickschat, J. S. Quorum sensing and bacterial biofilms. Nat. Prod. Rep. 27, 343-369 (2010).
3. Amara, N., Krom, B. P., Kaufmann, G. F. & Meijler, M. M. Macromolecular inhibition of quorum sensing: enzymes, antibodies, and beyond. Chem. Rev. 111, 195-208 (2011).
4. Bzdrenga, J. et al. Biotechnological applications of quorum quenching enzymes. Chem. Biol. Interact. 267, 104-115 (2017).
5. Fetzner, S. Quorum quenching enzymes. J. Biotechnol. 201, 2-14 (2015).
6. Hiblot, J., Gotthard, G., Chabriere, E. & Elias, M. Structural and Enzymatic characterization of the lactonase SisLac from Sulfolobus islandicus. PLoS ONE 7, (2012).
7. Draganov, D. I. et al. Human paraoxonases (PON1, PON2, and PON3) are lactonases with overlapping and distinct substrate specificities. J. Lipid Res. 46, 1239-1247 (2005).
8. Dong, Y.-H. et al. Quenching quorum-sensing-dependent bacterial infection by an N-acyl homoserine lactonase. Nature 411, 813-817 (2001).
9. LaSarre, B. & Federle, M. J. Exploiting Quorum Sensing To Confuse Bacterial Pathogens. Microbiol. Mol. Biol. Rev. MMBR 77, 73-111 (2013).
10. Liu, D. et al. Three-dimensional structure of the quorum-quenching N-acyl homoserine lactone hydrolase from Bacillus thuringiensis. Proc. Natl. Acad. Sci. U.S.A 102, 11882-11887 (2005).
11. Kim, M. H. et al. The molecular structure and catalytic mechanism of a quorum-quenching N-acyl-L-homoserine lactone hydrolase. Proc. Natl. Acad. Sci. U.S.A 102, 17606-17611 (2005).
12. Liu, D. et al. Mechanism of the Quorum-Quenching Lactonase (AiiA) from Bacillus thuringiensis. 1. Product-Bound Structures. Biochemistry (Mosc.) 47, 7706-7714 (2008).
13. Momb, J. et al. Mechanism of the quorum-quenching lactonase (AiiA) from Bacillus thuringiensis. 2. Substrate modeling and active site mutations. Biochemistry (Mosc.) 47, 7715-7725 (2008).
14. Liu, D. et al. Structure and specificity of a quorum-quenching lactonase (AiiB) from Agrobacterium tumefaciens. Biochemistry (Mosc.) 46, 11789-11799 (2007).
15. Mascarenhas, R. et al. Structural and Biochemical Characterization of AidC, a Quorum-Quenching Lactonase with Atypical Selectivity. Biochemistry (Mosc.) 54, 4342-4353 (2015).
16. Bergonzi, C., Schwab, M., Chabriere, E. & Elias, M. The quorum-quenching lactonase from Alicyclobacter acidoterrestris: purification, kinetic characterization, crystallization and crystallographic analysis. Acta Crystallogr. Sect. F 73, 476-480 (2017).
17. Bergonzi, C., Schwab, M. & Elias, M. The quorum-quenching lactonase from Geobacillus caldoxylosilyticus: purification, characterization, crystallization and crystallographic analysis. Acta Crystallogr. Sect. F Struct. Biol. Commun. 72, 681-686 (2016).
18. Kabsch, W. XDS. Acta Crystallogr. D Biol. Crystallogr. 66, 125-132 (2010).
19. Vagin, A. & Teplyakov, A. Molecular replacement with MOLREP. Acta Crystallogr. D Biol. Crystallogr. 66, 22-25 (2010).
20. Cowtan, K. The Buccaneer software for automated model building. 1. Tracing protein chains. Acta Crystallogr. D Biol. Crystallogr. 62, 1002-1011 (2006).
21. Emsley, P., Lohkamp, B., Scott, W. G. & Cowtan, K. Features and development of Coot. Acta Crystallogr. D Biol. Crystallogr. 66, 486-501 (2010).
22. Murshudov, G. N. et al. REFMAC5 for the refinement of macromolecular crystal structures. Acta Crystallogr. D Biol. Crystallogr. 67, 355-367 (2011).
23. Hiblot, J., Bzdrenga, J., Champion, C., Chabriere, E. & Elias, M. Crystal structure of VmoLac, a tentative quorum quenching lactonase from the extremophilic crenarchaeon Vulcanisaeta moutnovskia. Sci. Rep. 5, 8372 (2015).
24. Elias, M. et al. Structural Basis for Natural Lactonase and Promiscuous Phosphotriesterase Activities. J. Mol. Biol. 379, 1017-1028 (2008).
25. Bar-Rogovsky, H., Hugenmatter, A. & Tawfik, D. S. The evolutionary origins of detoxifying enzymes: the mammalian serum paraoxonases (PONs) relate to bacterial homoserine lactonases. J. Biol. Chem. 288, 23914-23927 (2013).
26. Bzdrenga, J. et al. SacPox from the thermoacidophilic crenarchaeon Sulfolobus acidocaldarius is a proficient lactonase. BMC Res. Notes 7, 333 (2014).
27. Clevenger, K. D., Wu, R., Er, J. A. V., Liu, D. & Fast, W. Rational Design of a Transition State Analogue with Picomolar Affinity for Pseudomonas aeruginosa PvdQ, a Siderophore Biosynthetic Enzyme. ACS Chem. Biol. 8, 2192-2200 (2013).
28. López, M. et al. Quorum sensing network in clinical strains of A. baumannii: AidA is a new quorum quenching enzyme. PloS One 12, e0174454 (2017).
29. Chow, J. Y., Yang, Y., Tay, S. B., Chua, K. L. & Yew, W. S. Disruption of Biofilm Formation by the Human Pathogen Acinetobacter baumannii Using Engineered Quorum-Quenching Lactonases. Antimicrob. Agents Chemother. 58, 1802-1805 (2014).
30. Hiblot, J., Gotthard, G., Chabriere, E. & Elias, M. Characterisation of the organophosphate hydrolase catalytic activity of SsoPox. Sci. Rep. 2, (2012).
31. Elias, M. & Tawfik, D. S. Divergence and convergence in enzyme evolution: parallel evolution of paraoxonases from quorum-quenching lactonases. J. Biol. Chem. 287, 11-20 (2012).
32. Afriat-Jurnou, L., Jackson, C. J. & Tawfik, D. S. Reconstructing a missing link in the evolution of a recently diverged phosphotriesterase by active-site loop remodeling. Biochemistry (Mosc.) 51, 6047-6055 (2012).
33. Ben-David, M. et al. Catalytic versatility and backups in enzyme active sites: the case of serum paraoxonase 1. J. Mol. Biol. 418, 181-196 (2012).
34. Vieille, C. & Zeikus, G. J. Hyperthermophilic enzymes: sources, uses, and molecular mechanisms for thermostability. Microbiol. Mol. Biol. Rev. MMBR 65, 1-43 (2001).
35. Liu, C. F. et al. A phenylalanine clamp controls substrate specificity in the quorum-quenching metallo-γ-lactonase from Bacillus thuringiensis. Biochemistry (Mosc.) 52, 1603-1610 (2013).
36. Chow, J. Y. et al. Directed evolution of a thermostable quorum-quenching lactonase from the amidohydrolase superfamily. J. Biol. Chem. 285, 40911-40920 (2010).
37. Tang, K. et al. MomL, a novel marine-derived N-acyl homoserine lactonase from Muricauda olearia. Appl. Environ. Microbiol. 81, 774-782 (2015).
38. Mascarenhas, R. et al. Structural and biochemical characterization of AidC, a quorum-quenching lactonase with atypical selectivity. Biochemistry (Mosc.) 54, 4342-4353 (2015).
39. Wang, L.-H., Weng, L.-X., Dong, Y.-H. & Zhang, L.-H. Specificity and enzyme kinetics of the quorum-quenching N-Acyl homoserine lactone lactonase (AHL-lactonase). J. Biol. Chem. 279, 13645-13651 (2004).
40. Safari, M., Amache, R., Esmaeilishirazifard, E. & Keshavarz, T. Microbial metabolism of quorum-sensing molecules acyl-homoserine lactones, γ-heptalactone and other lactones. Appl. Microbiol. Biotechnol. 98, 3401-3412 (2014).
41. Ceniceros, A., Dijkhuizen, L. & Petrusma, M. Molecular characterization of a Rhodococcus jostii RHA1 γ-butyrolactone (-like) signalling molecule and its main biosynthesis gene gblA. Sci. Rep. 7, 17743 (2017).
42. Holden, M. T. et al. Quorum-sensing cross talk: isolation and chemical characterization of cyclic dipeptides from Pseudomonas aeruginosa and other gram-negative bacteria. Mol. Microbiol. 33, 1254-1266 (1999).
43. Trovato, A. et al. Quorum vs. diffusion sensing: a quantitative analysis of the relevance of absorbing or reflecting boundaries. FEMS Microbiol. Lett. 352, 198-203 (2014).
44. Michael, B., Smith, J. N., Swift, S., Heffron, F. & Ahmer, B. M. SdiA of Salmonella enterica is a LuxR homolog that detects mixed microbial communities. J. Bacteriol. 183, 5733-5742 (2001).

### Example 2

### Creation of Improved Variants of Gel

In order to improve the properties of GcL, we used “ancestral mutations”. The use of ancestral mutations was previously reported to be useful in improving the solubility¹, the stability² or the activity of proteins³. The main advantage in the use of these mutations resides in the need for screening a low number of variants.

We collected 250 sequences homologous to GcL and aligned these sequences using MEGA5⁴, and subsequently manually improved. A phylogenetic tree was built from the obtained alignment using MEGA⁴. Based on this tree, one node comprising GcL sequence, as well as other homologous sequences sharing 70-75% sequence identity was selected. The most likely sequence at this node was reconstructed using MEGA5 and default parameters. The sequence is below:

>Ancestor1- node1
(SEQ ID NO: 10)
MTNIVKARPKLYVMDNGRMRMDKNWMIAMHNPATIHNPNAPTEFVEFPIYT
VLIDHPEGKILFDTACNPNSMGPQGRWAEATQQMFPWTASEECYLHNRLEQ
LKVRPEDIKFVVASHLHLDHAGCLEMFTNATIIVHEDELNGTLQCYARNQK
EGAYIWADIDAWIKNNLQVRTIKRHEDNLLLAEGIRVLNFGSGHAWGMLGL
HVELPETGGIILASDAIYTAESYGPPIKPPGIIYDSLGYVNTVEKIRRIAK
ETNSQVWFGHDAEQFKQFRKSTEGYYE

Reconstructed ancestral sequences are enriched in conserved residues. Indeed, one fundamental phylogeny principle considers that if a position is conserved, it is likely to be ancestral. We aligned the sequence of GcL “wild-type” with the reconstructed ancestral sequences for the selected node. Discrepancies between sequences suggests mutations to introduce into the wild type GcL sequences. Here, a total of 20 discrepancies were observed, and constitute our pool of ancestral mutations.

We decided to use our structural data and structural interpretation to decrease the number of mutations and identify the proper combination of mutations to improve GcL's properties, particularly its solubility and activity levels. Therefore, we used four distinct criteria to identify key combinations of mutations:
Retain substitutions of surface apolar to polar residues
Retain substitutions of Gly to X, except when located at the start or end of secondary structures
Include core mutations
Include changes of surface hydrophobic to less hydrophobic residues

Only ancestral mutations corresponding to one or more of these criteria were retained (Table 5). Therefore, we obtained a combination of 14 mutations. A careful structural examination and analysis allowed to reduce this list to 4 mutations that appeared to make key interactions within the protein structure.

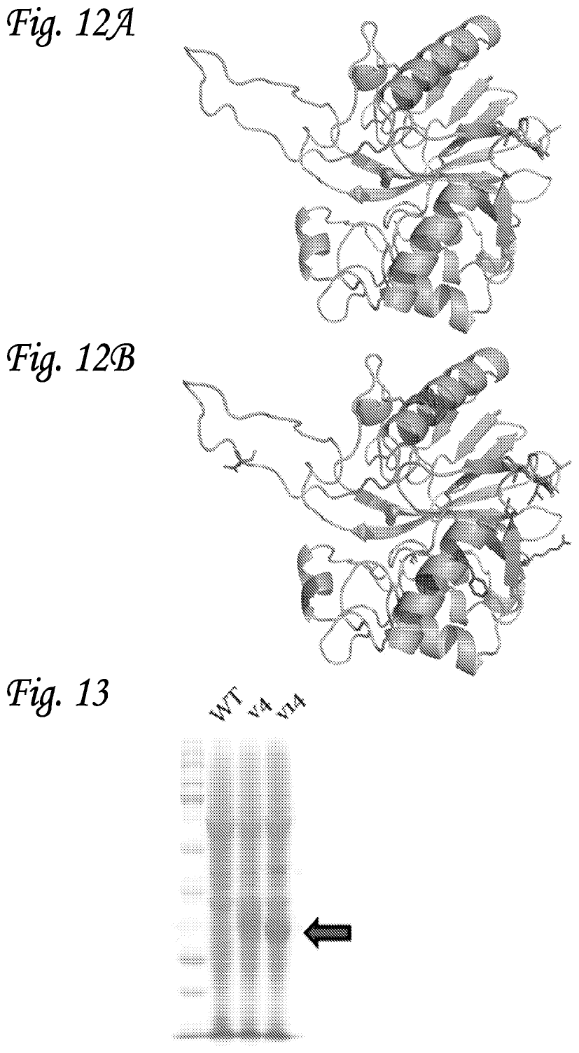

TABLE 5
List of mutations retained for the GcL 4 and GcL 14 variants.
Common mutations to both constructs are bolded. Mutations
are shown in FIG. 12.

| Mutations | Location | GcL 4 | GcL 14 |
|---|---|---|---|
| Q41P | Dimerization loop | | X |
| S66A | buried | | X |
| S81A | Surface | | X |
| T91S | Surface | | X |
| R111K | Surface | | X |
| F141 | Surface | | X |
| A144T | slightly buried | | X |
| C147S | buried | X | X |
| V1751 | slightly buried | | X |
| H178D | buried | X | X |
| I182L | Surface | | X |
| L183E | partly buried | X | X |
| M244A | Surface | X | X |
| N245K | Surface | | X |

Both genes were synthesized by GenScript (Piscataway, N.J., USA). Synthetic genes were fused to a N-ter STREP-tag (WSTIPQFEK (SEQ ID NO: 8)) for affinity purification, followed by a TEV cleavage site (ENLYFQS (SEQ ID NO:9)) allowing for the removal of this tag, leaving only a N-ter Ser residue after cleavage.

### Heterologous Expression Levels

The expression of GcL wt is much less important than for GcL 4 and GcL 14. In fact, the engineered enzyme shows after induction a higher expression level (FIG. 13). Moreover after purification the quantities of enzyme produced differs. The quantity of enzyme produce after purification is 100 mg/3 L whereas it produces 1 g/3 L for GcL 4 and 14.

Thermal stability of GcL and the mutants.

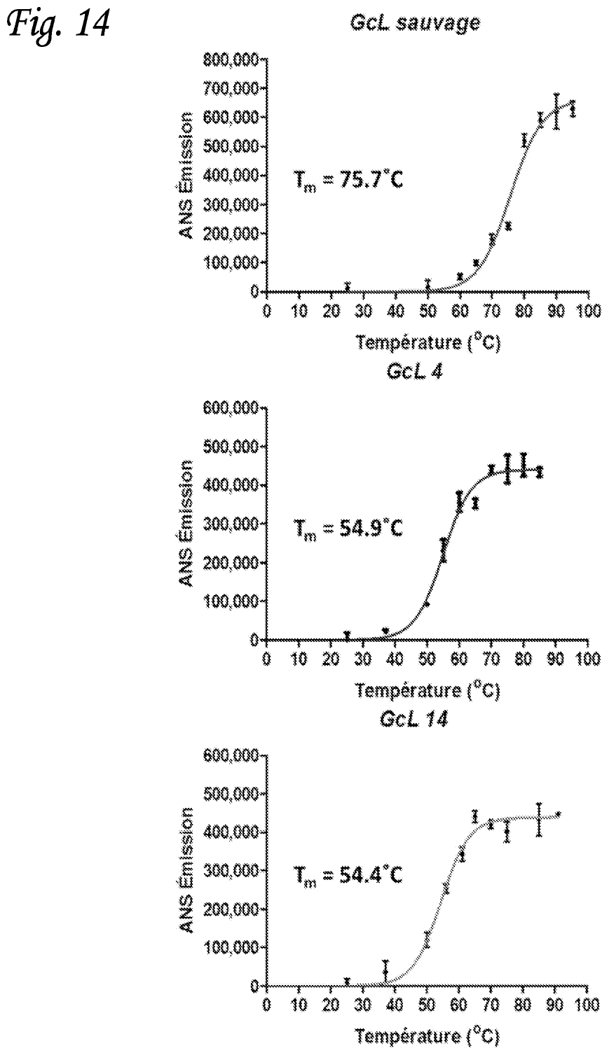

The thermal stability of the enzymes against heat was determined using the ANS (8-Anilinonaphthalene-1-sulfonic acid) fluorescence thermal shift assay (FIG. 14) (Hawe et al., 2008). Triplicate samples (250 μl) containing 2.5 μM of pure enzyme and 10 μM of ANS were prepared. The samples were vortexed and incubated for 30 minutes at 25, 37, 50, 60, 65, 70, 75, 80, 85, 90 and 95° C. in different heating blocks. Then, samples were assayed in a black, 96-well flat bottom plate (Flat bottom 96 well, Fisherbrand) and measured using a fluorescence microplate reader (Synergy HTX, BioTek, USA) with GEN5.1 software, using an excitation wavelength at 360 nm and an emission wavelength at 508 nm. The melting temperature of the enzyme (Tₘ), defined here as the temperature at which 50% of the maximal ANS fluorescence is reached, was determined by fitting the ANS fluorescence signal to the following equation at different tested temperatures using the GraphPad Prism software.

$$Y = Bottom + \frac{( Top - Bottom )}{1 + exp ( \frac{Tm - X}{h} )} \qquad ( 1 )$$

Where X, Y, and h represent the incubation temperature, the ANS fluorescence, and the slope coefficient, respectively.
We note here that both variants GcL 4 and GcL 14 exhibit a lower melting temperature than GcL wild-type. Therefore, selected mutations might have a destabilizing effect on the enzyme. Remarkably, both variants GcL 4 and GcL 14 behave nearly identically, and mutations responsible for this loss in thermal stability might therefore be common.

### Enzymatic Characterization of GcL 4 and GcL 14.

We determined the ability of these two variants to hydrolyze a wide range of lactones as substrates, using the assay described in the previous chapter. GcL 4 was assayed against substrates C4 AHIL, C8 AHIL, 3-oxo-C8 AHL, 3-oxo-C12 AHL, γ-Butyrolactone, γ-Nonalactone, γ-Decanolactone, δ-Valerolactone, δ-Octanolactone, δ-Decalactone, ε-Caprolactone, ε-Decalactone and Whiskey Lactone, while GcL 144 was assayed against C4 AHL, C8 AHL, 3-oxo-C8 AHL, 3-oxo-C12 AHIL, γ-Butyrolactone, γ-Heptalactone, γ-Nonalactone, γ-Decanolactone, δ-Valerolactone, δ-Nonalactone, δ-Decalactone, ε-Caprolactone, ε-Decalactone and Whiskey Lactone (Table 6).

TABLE 6.
Kinetic studies of variants GcL 4 and GcL 14.

| | GcL 4 | | | GcL 14 | | |
|---|---|---|---|---|---|---|
| | k_{cat} (s⁻¹) | k_{M} (μM) | k_{cat}/k_{M} (s⁻¹M⁻¹) | k_{cat} (s⁻¹) | k_{M} (μM) | k_{cat}/k_{M} (s⁻¹M⁻¹) |
| C4 AHL | 18.25 ± 0.85 | 0.75 ± 0.24 | (2.44 ± 0.82) × 10⁷ | 15.01 ± 0.61 | 0.92 ± 0.27 | (1.63 ± 0.48) × 10⁷ |
| C8 AHL | 18.31 ± 0,74 | 0.81 ± 0.20 | (2.26 ± 0.57) × 10⁷ | 18.38 ± 0.86 | 1.32 ± 0.34 | (1.40 ± 0.37) × 10⁷ |
| 3-oxo-C8 AHL | 76.4 ± 3.87 | 1.09 ± 0.28 | (7.01 ± 1.86) × 10⁷ | 76.00 ± 4.04 | 0.29 ± 0.08 | (2.65 ± 0.79) × 10⁸ |
| 3-oxo-C12 AHL | nm | nm | nm | 15.88 ± 0.45 | 0.50 ± 0.12 | (3.17 ± 0.77) × 10⁷ |
| γ-Butyrolactone | 8.42 ± 0.42 | 1.99 ± 0.45 | (4.23 ± 0.98) × 10⁶ | 9.89 ± 0.75 | 5.90 ± 1.67 | (1.68 ± 0.49) × 10⁶ |
| γ-Heptalactone | nm | nm | nm | 20.95 ± 0.93 | 2.77 ± 0.53 | (7.57 ± 1.49) × 10⁶ |
| γ-Nonalactone | 18.3 ± 0.90 | 2.54 ± 0.97 | (6.94 × 2.50) × 10⁶ | 23.61 ± 1.40 | 9.09 ± 2.20 | (2.60 ± 0.64) × 10⁶ |
| γ-Decanolactone | 22.24 ± 1.10 | 1.31 ± 0.28 | (1.69 ± 0.38) × 10⁷ | 13.7 ± 0.73 | 0.32 ± 0.14 | (4.23 ± 1.90) × 10⁷ |
| δ-Valerolactone | 11.94 ± 1.03 | 3.13 ± 0.10 | (3.8 2 ± 1.20) × 10⁶ | 22.13 ± 0.41 | 1.12 ± 0.12 | (1.97 ± 0.22) × 10⁷ |
| δ-Octanolactone | 22.01 ± 1.17 | 1.92 ± 0.54 | (1.15 ± 3.29) × 10⁷ | nm | nm | nm |
| δ-Nona1actone | nm | nm | nm | 23.61 ± 1.40 | 9.09 ± 2.20 | (2.60 ± 0.64) × 10⁶ |
| δ-Decalactone | 10.28 ± 0.76 | 5.54 ± 0.13 | (1.86 ± 4.59) × 10⁶ | 13.7 ± 0.73 | 0.32 ± 0.14 | (4.23 ± 1.90) × 10⁷ |
| ε-Caprolactone | 12.63 ± 0.95 | 1.30 ± 0.49 | (9.71 ± 3.76) × 10⁶ | 23.74 ± 1.26 | 2.49 ± 0.67 | (9.55 ± 2.62) × 10⁶ |
| ε-Decalactone | 6.3 ± 0.29 | 1.41 ± 0.30 | (4.46 ± 0.98) × 10⁶ | 10.41 ± 0.85 | 7.98 ± 2.47 | (1.30 ± 0.41) × 10⁶ |
| Whiskey Lactone | 8.4 ± 0.39 | 1,41 ± 0.31 | (5.94 ± 1.32) × 10⁶ | 7.98 ± 0.63 | 14.00 ± 3.74 | (5.71 ± 1.59) × 10⁵ |

nm, not measured.

Kinetic data reveals that GcL 4 and GcL 14 are extremely proficient lactonases. Remarkably, their kinetic parameters are very similar, consistent with their similar biochemical and expression properties. Kinetic data reveal that these enzymes exhibit K_{M} values lower than GcL wt. This is particularly evidenced by the K_{M} values for C4 AHL (˜1 μM) while the wt enzyme has a K_{M} value of ˜230 μM. Catalytic proficiencies of the two variants and the wt enzyme are similar for γ-Nonalactone, δ-Decalactone, whiskey lactone and ε-Decalactone. However, catalytic proficiencies of variants GcL 4 and GcL 14 are increased by 1 to 3 orders of magnitude for substrates C4 AHL, C8 AHL, 3-oxo-C8 AHL, γ-Butyrolactone, γ-Décanolactone, δ-Valérolactone and ε-Caprolactone, as compared to the wt enzyme. It is of note that the catalytic efficiency of GcL 14 against 3-oxo-C8 AHL (k_{cat}/kM=2.65×108 s⁻¹M⁻¹) makes this enzyme the most active lactonase ever characterized.

### Preliminary Structural Characterization of the Variants GcL 4 and GcL 14.

Variants were crystallized in similar conditions than the wt enzyme. Data were collected at the synchrotron APS Argonne (23IDD, Lemont, Ill., USA). Diffraction data were processed as described for the wt enzyme (Table 7).

TABLE 7
Data Collection parameters for GcL 4 and GcL 14 mutants.

| | | soaking/ | | Space | |
|---|---|---|---|---|---|
| Variant | Resolution | co-crystallization | Substrate | group | Unit cell parameters |
| GcL 4 | 1.9 Å | none | none | C2 | a = 145.92, b = 110.91, c = 79.34 |
| | | | | | α = γ = 90.000, β = 116.535 |
| GcL 14 | 2.2 Å | none | none | C2 | a = 162.06, b = 109.73, c = 97.81 |
| | | | | | α = γ = 90.000, 116.612 |
| GcL 14 | 1.7 Å | Co-crystallization | C4 AHL | C2 | a = 161.35, b = 109.61, c = 97.55 |
| | | | | | α = γ = 90.000, β = 116.157 |
| Gc L14 | 2.25 Å | soaking | C4 AHL | C2 | a = 161.13, b = 109.33, c = 97.45 |
| | | | | | α = γ = 90.000, β = 116.166 |
| GcL 14 | 2.45 Å | soaking | γ-dodecalactone | C2 | a = 160.30, b = 108.78, c = 97.08 |
| | | | | | α = γ = 90.000, β = 116.771 |
| GcL 14 | 2.35 A | soaking | γ-nonalactone | C2 | a = 159.84, b = 107.93, c = 96.42 |
| | | | | | α = γ = 90.000, β = 116.691 |

Using these data, we could solve the structures of GcL 4 and GcL 14 in complex with various lactone substrates, by using soaking and co-crystallization strategies. Structures are currently being refined and analyzed.

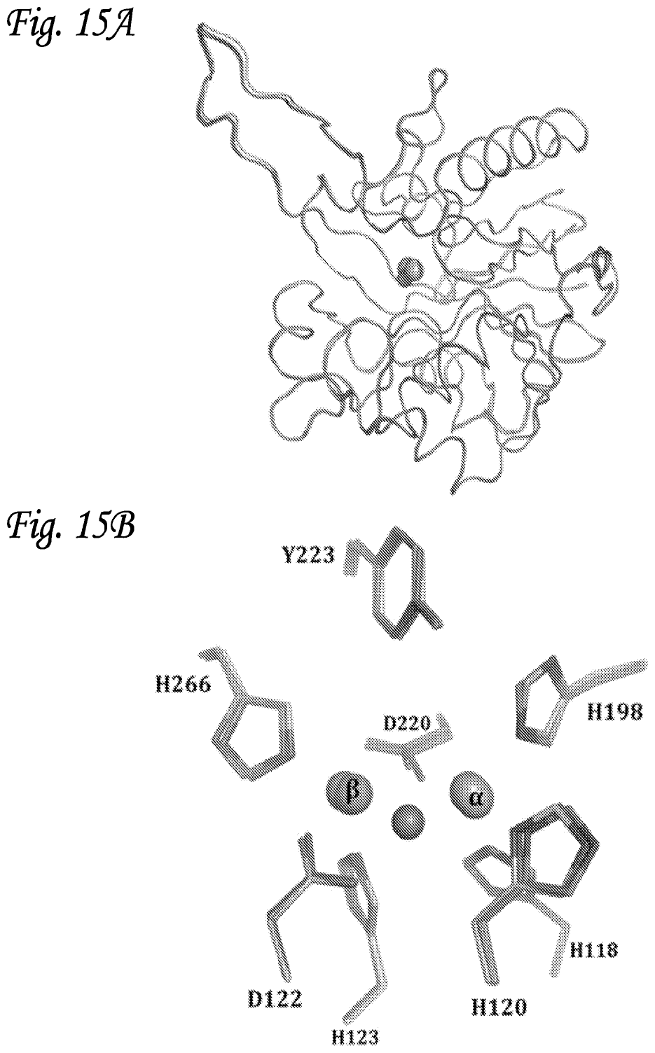

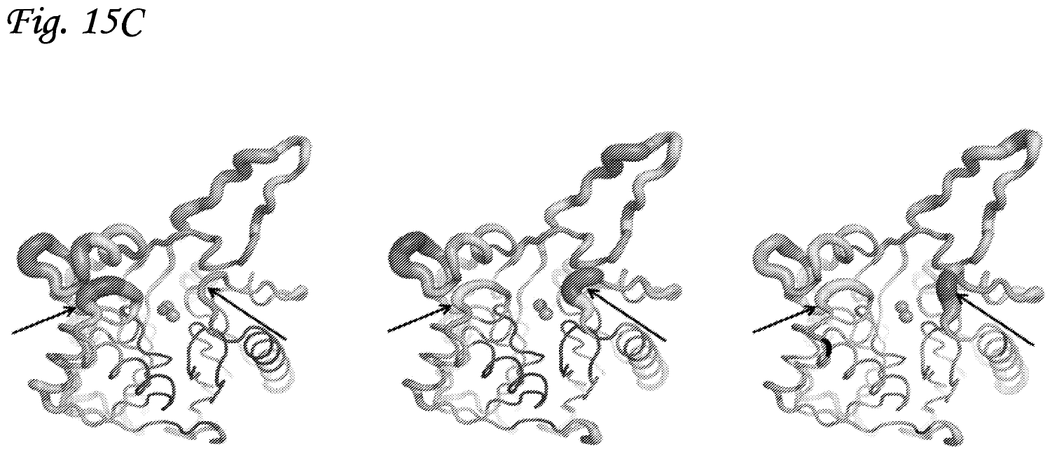

Preliminary analysis of these structures reveals that the structures of both variants are extremely similar to the structure of the wt enzyme (FIGS. 15A and 15B). This is intriguing in the light of the large difference in catalytic efficiency of these different enzymes.

A preliminary analysis of the active sites' loops mobility, using the thermal motion B-factor, highlights differences. Indeed, active sites' loop that are rigid in the wt enzyme are disordered in the two variants structure, and another active site loop undergoes the opposite change: mobile in the wt enzyme, rigid in the mutants' structures (FIG. 15C). These changes in the mobility of active sites' loop was previously related to changes in catalytic efficiency of enzymes⁵, and particularly of lactones⁶. We propose to further examine our structural data, including structures in complex with lactone substrates, and to elucidate the structural determinants accounting for the increase in catalytic efficiency for both GcL 4 and GcL 14.

Structural Data in Complex with Other Lactone Substrates

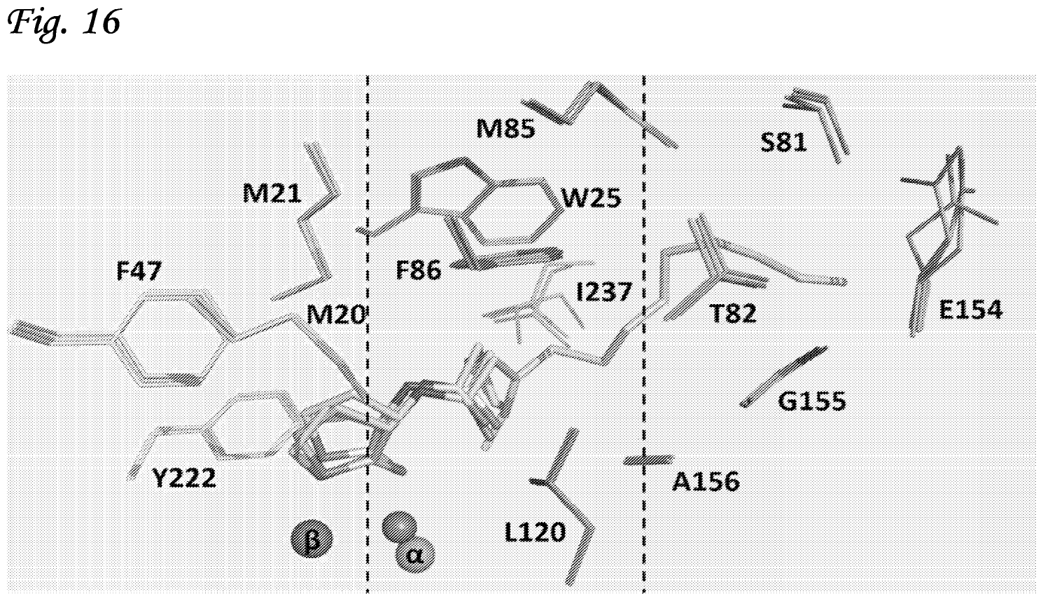

From our analysis of the structures of GcL obtained with different lactones (C4-HSL, C6-HSL and 3-oxo C12 HSL), we identified M21, Y222, F47, W25, F86, A156, L120, M85, G155, T82, 581, E154 and 1236 as key residues interacting with the lactone substrates. Structural data show that these positions are relevant to the enzyme activity and its substrate specificity (FIG. 16).

Library construction and screening: Site saturation mutagenesis libraries for positions identified by structural analysis were ordered from Genscript (Piscataway, N.J.), and cloned in pET22b vectors. The library glycerol stocks were used to inoculate a starter of E. coli BL21 culture cells overnight at 37° C. The culture was used to inoculate ZYP media in a 96-well plate format, and the plate was incubated at 450 rpm, 37° C. for 4 hours. Temperature transition to 18° C. and addition of 1 mM of cobalt chloride (final) was performed and culture allowed to grow for 16 hours. Plate was centrifuged for 10 min at 4,000 rpm. Cell lysis was performed on ice for 45 min using 295 μL of Bug Buster, 1.5 μL of lysozyme (200 mg/mL stock), 0.50 μL of DNAse and 3 μL of PMSF in each well. Cell lysate were transferred to microfuge tube and centrifuged at 14,000 g at 4° C.

Supernatants were assayed for acylhomocysteine activity in PTE Buffer pH 8.0 containing 50 mM HEPES, 150 mM NaCl, 0.2 mM CoCl2, 2 mM Ellman reagent and 3 mM (or 1 mM) of substrate. Three different substrates were used for the screening, C1-, C4- and C8 homocysteine thiolactones. Reaction was monitored at 412 nm over a 30 min timespan. Reading were performed in triplicates

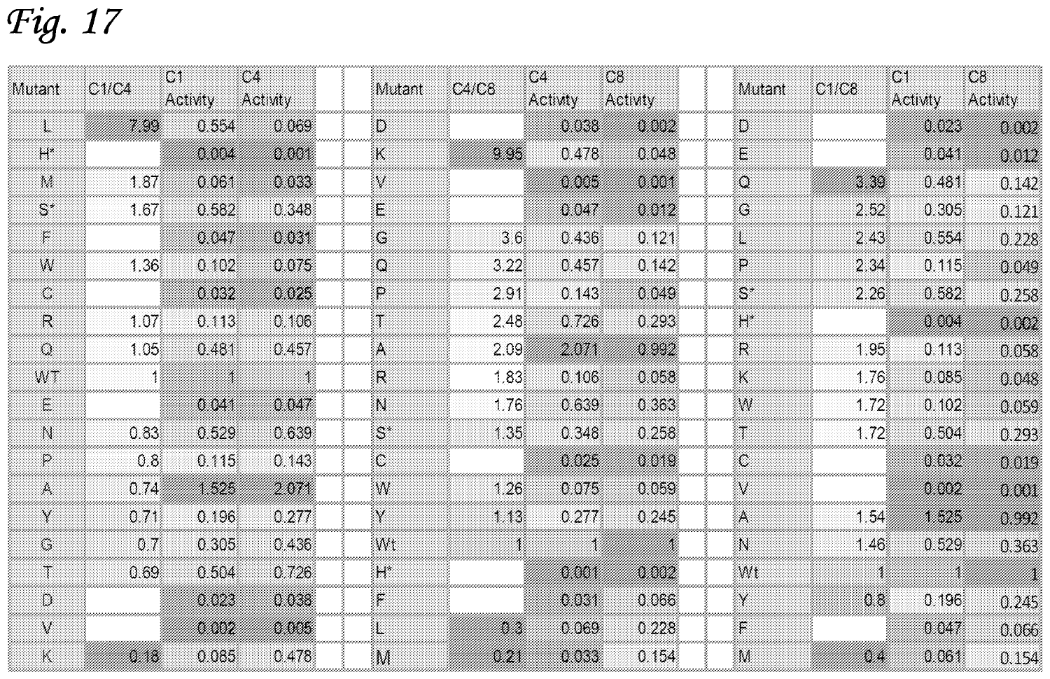

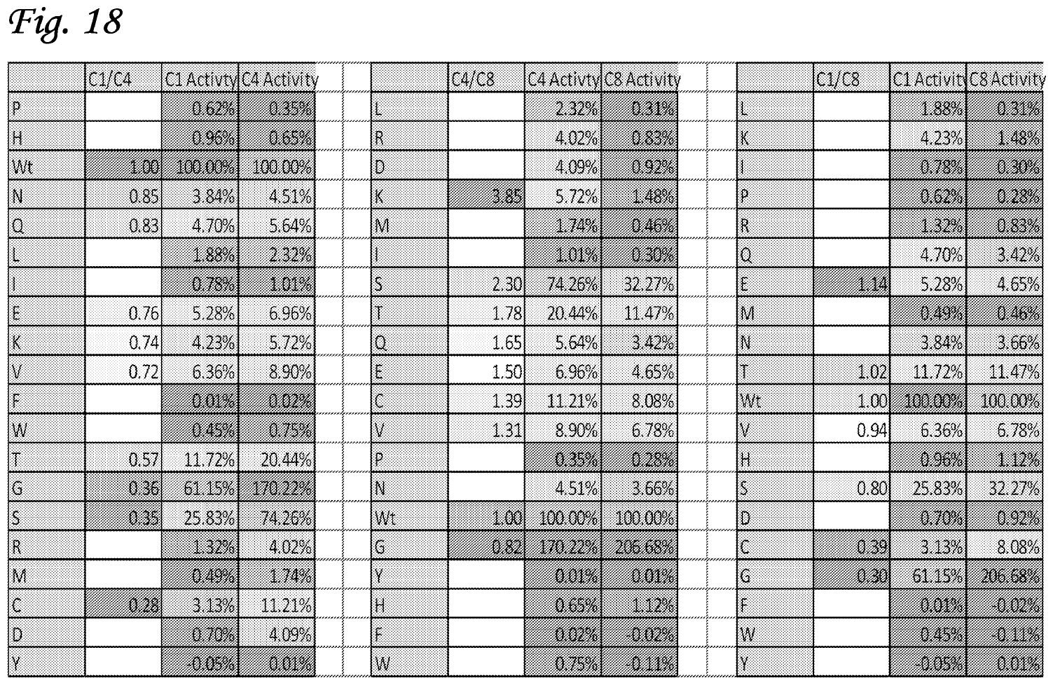

These screenings (FIGS. 17 and 18) reveal that some mutants exhibit increased activity levels, i.e. I236A and A156G. Other mutants have altered substrate preferences, such as I236 L, M, K, S, T, G, Y and A156T, G, S, Q, E, K.

### Conclusions

We have created, combining phylogeny derived mutations and our structural analysis, variants of GcL that exhibit lower thermal stability, but higher solubility and expression levels in E. coli. Additionally, these variants, GcL 4 and GcL 14, possesses largely increased catalytic efficiencies in the hydrolysis of numerous lactones as substrates. GcL 14 is, by far, the most active lactonase characterized to date. Citations for Example 2
1. Gonzalez, D. et al. Ancestral mutations as a tool for solubilizing proteins: The case of a hydrophobic phosphate-binding protein. FEBS Open Bio 4, 121-7 (2014).
2. Dellus-Gur, E., Toth-Petroczy, A., Elias, M. & Tawfik, D. S. What makes a protein fold amenable to functional innovation? Fold polarity and stability trade-offs. J Mol Biol 425, 2609-21 (2013).
3. Alcolombri, U., Elias, M. & Tawfik, D. S. Directed evolution of sulfotransferases and paraoxonases by ancestral libraries. J Mol Biol 411, 837-53 (2011).
4. Tamura, K. et al. MEGA5: molecular evolutionary genetics analysis using maximum likelihood, evolutionary distance, and maximum parsimony methods. Mol. Biol. Evol. 28, 2731-2739 (2011).
5. Dellus-Gur, E. et al. Negative Epistasis and Evolvability in TEM-1 beta-Lactamase—The Thin Line between an Enzyme's Conformational Freedom and Disorder. J Mol Biol 427, 2396-409 (2015).
6. Hiblot, J., Gotthard, G., Elias, M. & Chabriere, E. Differential active site loop conformations mediate promiscuous activities in the lactonase SsoPox. PLoS One 8, e75272 (2013).

### Example 3

### Creation of Improved Mutants of a Quorum Quenching Lactonase SsoPox

SsoPox is a well-characterized lactonase that that was been engineered. It presents the advantages to be more stable than GcL-like lactonases but is much more stable towards heat, chemicals, and other stress factors.

In order to improve the properties of Ssopox, we used “ancestral mutations”. The use of ancestral mutations was previously reported to be useful in improving the solubility¹, the stability² or the activity of proteins³. The main advantage in the use of these mutations resides in the need for screening a low number of variants.

We collected a total of 150 sequences homologous to Ssopox (Q97VT7), DrOPH (Q9RVU2) and the PTE from B. diminuta (P0A433) using BLAST. Redundant sequences were removed using CD-HIT and a 0.9 cutoff. Multiple sequence alignment was performed using MUSCLE and aligned these sequences using MEGA5⁴, and subsequently manually improved. A phylogenetic tree was built from the obtained alignment using MEGA⁴. Based on this tree, one node comprising Ssopox sequence, as well as other homologous sequences sharing 70-75% sequence identity was selected. Reconstructed ancestral sequences are enriched in conserved residues. Indeed, one fundamental phylogeny principle considers that if a position is conserved, it is likely to be ancestral. We aligned the sequence of Ssopox “wild-type” with the reconstructed ancestral sequences for the selected node. Discrepancies between sequences suggests mutations to introduce into the wild type Ssopox sequence.

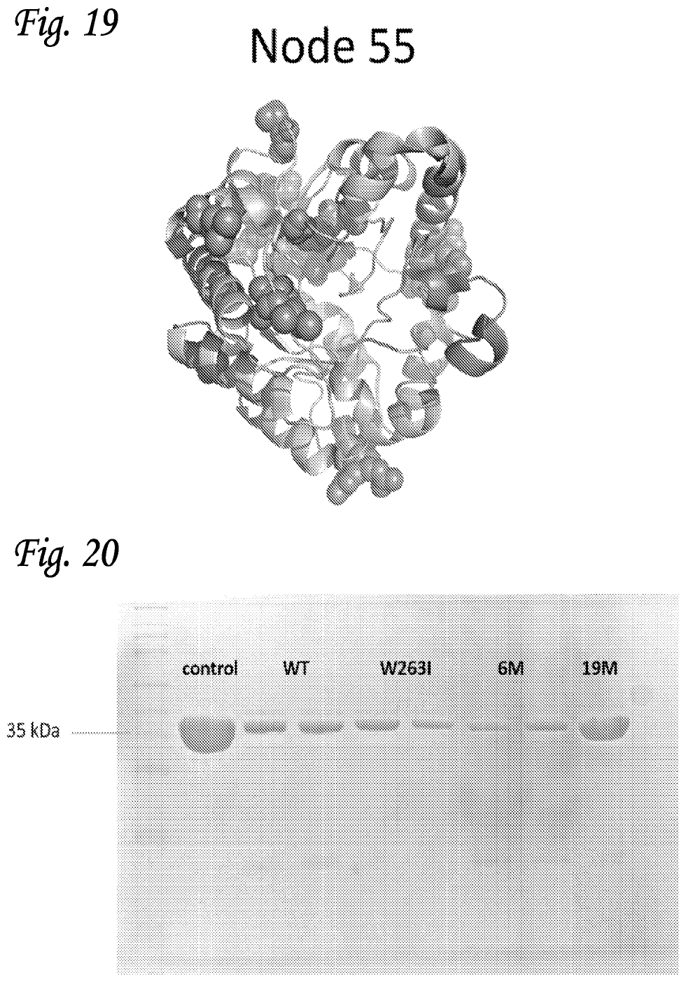

The predicted ancestral sequence at node 55 exhibited 20 substitutions as compared to the Ssopox wt sequence (FIG. 19): M1T/R2K/S10E/S13P/K14R/D15E/I16M/R55T/Q58S/F59Y/L90V/V91I/93A/I100T/L107N/L1 30N/I138V/N160H/T186M/R241K. The most likely sequence at this node was reconstructed using MEGA5 and default parameters.

>sp|Q97VT7|PHP₋SULSO Aryldialkylphosphatase OS =
Sulfolobus solfataricus (strain ATCC 35092 / DSM
1617 / JCM 11322 / P2) GN = php PE = 1 SV = 1
(SEQ ID NO: 11)
MRIPLVGKDSIESKDIGFTLIHEHLRVFSEAVRQQWPHLYNEDEEFRNAVN
EVKRAMQFGVKTIVDPTVMGLGRDIRFMEKVVKATGINLVAGTGIYIYIDL
PFYFLNRSIDEIADLFIHDIKEGIQGTLNKAGFVKIAADEPGITKDVEKVI
RAAAIANKETKVPIITHSNAHNNTGLEQQRILTEEGVDPGKILIGHLGDTD
NIDYIKKIADKGSFIGLDRYGLDLFLPVDKRNETTLRLIKDGYSDKIMISH
DYCCTIDWGTAKPEYKPKLAPRWSITLIFEDTIPFLKRNGVNEEVIATIFK
ENPKKFFS

We decided to use our structural data on SsoPox and structural interpretation to decrease the number of mutations and identify the proper combination of mutations to improve Ssopox's properties, particularly its solubility and activity levels. Therefore, we used four distinct criteria to identify key combinations of mutations:
Retain substitutions of surface apolar to polar residues
Retain substitutions of Gly to X, except when located at the start or end of secondary structures
Include core mutations
Include changes of surface hydrophobic to less hydrophobic residues

TABLE 8
Analysis of the predicted ancestral mutations for the two considered
nodes. A areful structural examination and analysis allowed to
reduce this list to 19 and 6 mutation that appeared to make
key interactions within the protein structure.

| Mutation | Location | Initial residue | Substitution |
|---|---|---|---|
| M1T | surface | hydrophobic | polar |
| R2K | surface | charged+ | charged+ |
| S10E | surface | polar | charged− |
| S13P | surface | polar | |
| K14R | surface | charged+ | charged+ |
| D15E | surface | charged− | charged− |
| I16M | close to | hydrophobic | hydrophobic |
| | surface | | |
| R55T | surface | charged+ | polar |
| Q58S | surface | polar | polar |
| F59Y | surface | hydrophobic/aromatic | hydrophobic/aromatic |
| L90V | buried | hydrophobic | hydrophobic/small |
| V91I | buried | hydrophobic/small | hydrophobic |
| G93A | buried | small | hydrophobic/small |
| I100T | surface | hydrophobic | polar |
| L107N | surface | hydrophobic | polar |
| L130N | surface | hydrophobic | polar |
| I138V | buried | hydrophobic | hydrophobic/small |
| | (next to | | |
| | KCX) | | |
| N160H | surface | polar | charged+ |
| T186M | surface | polar | hydrophobic |
| R241K | surface | charged+ | charged+ |

List of mutations retained for the SsoPox 6 and SsoPox 19 variants.
Node 55 selected mutations: 6 mutations
R2K/S10E/S13P/K14R/V91I/L107N
>Node55 selected mutations, shown as underlined residues:

(SEQ ID NO: 12)
MKIPLVGKDEIEPRDIGFTLIHEHLRVFSEAVRQQWPHLYNEDEEFRNAVN
EVKRAMQFGVKTIVDPTVMGLGRDIREMEKVVKATGINLIAGTGIYIYIDL
PFYFNNRSIDEIADLFIHDIKEGIQGTLNKAGFVKIAADEPGITKDVEKVI
RAAAIANKETKVPIITHSNAHNNTGLEQQRILTEEGVDPGKILIGHLGDTD
NIDYIKKIADKGSFIGLDRYGLDLFLPVDKRNETTLRLIKDGYSDKIMISH
DYCCTIDWGTAKPEYKPKLAPRWSITLIFEDTIPFLKRNGVNEEVIATIFK
ENPKKFFS

Node 55 full mutations (except M1): 19 mutations
R2K/S10E/S13P/K14R/D15E/I16M/R55T/Q58S/F59Y/L90V/V91I/G93A/I100T/L107N/L130N/I138V/N160H/T186M/R241K)
>Node55 mutations, shown as underlined residues:

(SEQ ID NO: 13)
MKIPLVGKDEIEPREMGFTLIHEHLRVFSEAVRQQWPHLYNEDEEFRNAVN
EVKTAMSYGVKTIVDPTVMGLGRDIRFMEKVVKATGINVIAATGIYIYTDL
PFYFNNRSIDEIADLFIHDIKEGIQGTNNKAGFVKVAADEPGITKDVEKVI
RAAAIAHKETKVPIITHSNAHNNTGLEQQRILMEEGVDPGKILIGHLGDTD
NIDYIKKIADKGSFIGLDRYGLDLFLPVDKRNETTLKLIKDGYSDKIMISH
DYCCTIDWGTAKPEYKPKLAPRWSITLIFEDTIPFLKRNGVNEEVIATIFK
ENPKKFFS

Both genes were synthesized by GenScript (Piscataway, N.J., USA). Production and purification of the enzyme was performed as previously described by our team

### Heterologous Expression Levels.

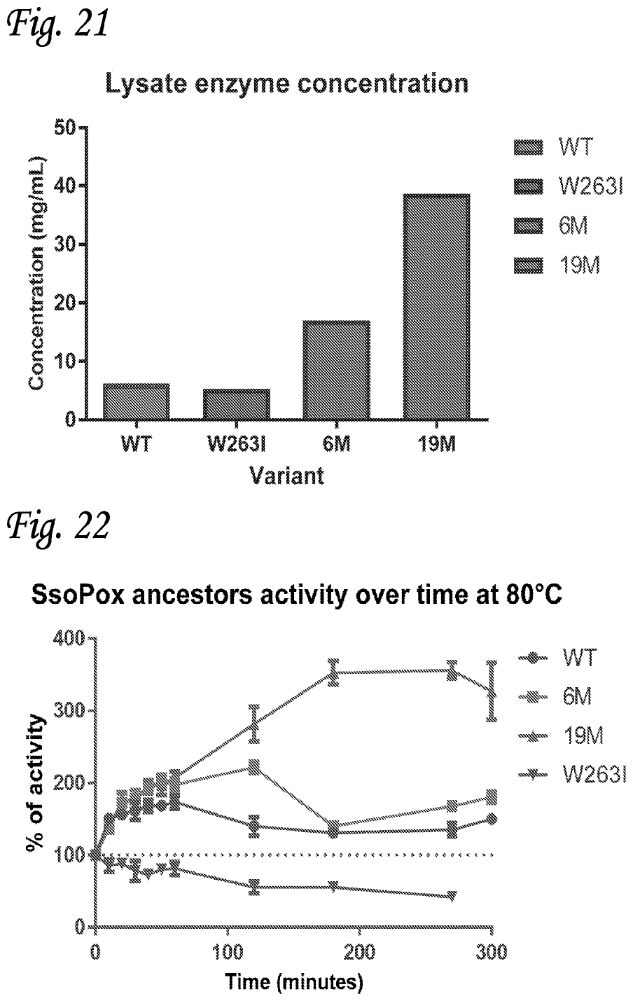

The expression of SsoPox wt is much less important than for SsoPox 6 and SsoPox 19 variants. In fact, the engineered enzyme shows after induction a higher expression level (see FIGS. 20 and 21). Moreover, after purification the quantities of enzyme produced differs. The quantity of enzyme produced is 2-fold and 4-fold higher for SsoPox 6 and SsoPox 19 variants as compared to SsoPox wt, respectively.

### Activity of SsoPox Variants at High Temperature Over Time.

We measured the ability of the variants to hydrolyze the phosphotriester paraoxon after incubation of different times at 80° C., as previously described⁶. We note here that variants SsoPox 6 and SsoPox 19 are withstanding better the incubation at 80° C. than the wt enzyme and SsoPox W263I used for references (FIG. 22).

### Enzymatic Characterization of SsoPox 6 and SsoPox 19.

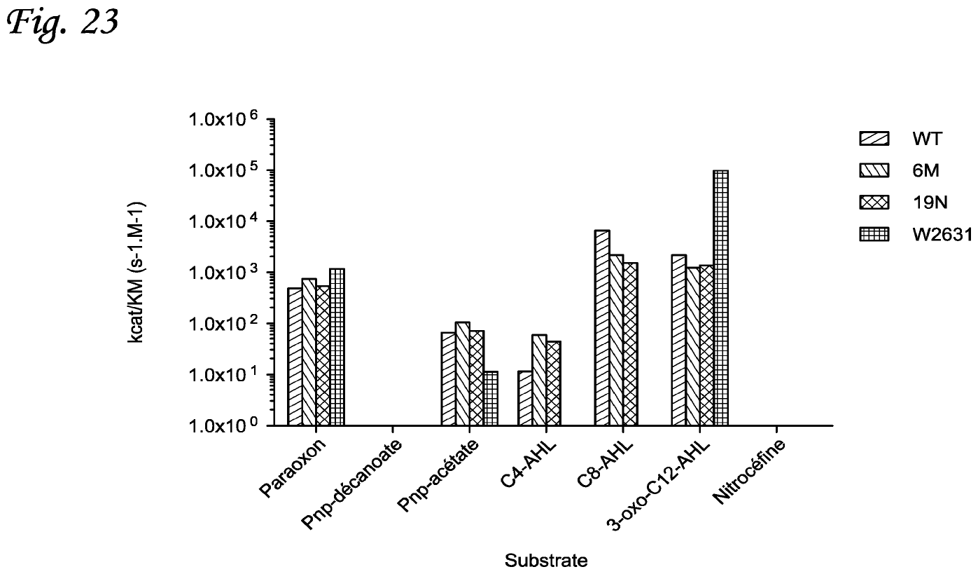

We determined the ability of these two variants to hydrolyze a wide range of substrates, including esters, lactones and phosphotriesters (FIG. 23). Overall, we note that both variants are catalytically active, and their activity levels are extremely similar to those of the wt enzyme.

### Conclusions

The variants SsoPox 6 and SsoPox 19 have higher expression and purification yields than SsoPox wt and other reference mutants (W263I). Moreover, they are also more active at high temperature, and withstand better temperature than SsoPox wt and W263I. They represent a major advance in the obtaining of robust lactonase for scale-up production while minimizing costs, as well as industrial process and functionalization to materials (heat treatment).

### Citations for Example 3

1. Gonzalez, D. et al. Ancestral mutations as a tool for solubilizing proteins: The case of a hydrophobic phosphate-binding protein. FEBS Open Bio 4, 121-7 (2014).
2. Dellus-Gur, E., Toth-Petroczy, A., Elias, M. & Tawfik, D. S. What makes a protein fold amenable to functional innovation? Fold polarity and stability trade-offs. J Mol Biol 425, 2609-21 (2013).
3. Alcolombri, U., Elias, M. & Tawfik, D. S. Directed evolution of sulfotransferases and paraoxonases by ancestral libraries. J Mol Biol 411, 837-53 (2011).
4. Tamura, K. et al. MEGA5: molecular evolutionary genetics analysis using maximum likelihood, evolutionary distance, and maximum parsimony methods. Mol. Biol. Evol. 28, 2731-2739 (2011).
5. Hiblot, J., Gotthard, G., Elias, M. & Chabriere, E. Differential active site loop conformations mediate promiscuous activities in the lactonase SsoPox. PLoS One 8, e75272 (2013).
6. Hiblot, J., Gotthard, G., Chabriere, E. & Elias, M. Characterisation of the organophosphate hydrolase catalytic activity of SsoPox. Sci Rep 2, (2012).
7. Jacquet, P. et al. Rational engineering of a native hyperthermostable lactonase into a broad spectrum phosphotriesterase. Sci. Rep. 7, 16745 (2017).

### Example 4

### Inhibition of Biocorrosion

### Abstract

Microbial colonization of steel surfaces can be detrimental to the integrity of metal surfaces and can lead to biocorrosion. Biocorrosion is a serious problem for aquatic and marine industries. In Minnesota (USA), where this study was conducted, biocorrosion severely affects the maritime transportation industry. Here, we investigated the anticorrosion activity of a variety of chemical (magnesium peroxide), biological (surfactin, capsaicin, and gramicidin), and enzymatic (a quorum quenching lactonase) bioactive coating additives. Experimentally coated steel coupons were submerged in Lake Superior water for two months. Biocorrosion was evaluated by counting the number and the coverage of corrosion tubercles on coupons, and also by performing SEM imaging of the coupon surface. Results show that three experimental coating additives significantly reduced the formation of corrosion tubercles: surfactin, magnesium peroxide and lactonase by 31%, 36% and 50%, respectively. Additionally, 16s rDNA sequencing analysis reveal that the decrease in corrosion is associated with a change in the composition of the microbial community at the surface of the steel. The remarkable performance of the coating containing the highly stable, quorum quenching enzyme will be further evaluated and may provide a biological, reliable, and cost-effective method to treat steel structures.

### INTRODUCTION

Microorganisms are highly capable of colonizing surfaces of numerous and diverse materials. This colonization process yields to a firmly adhering and complex microbial community termed biofilm [1]. Biofilm, which can lead to biofouling, are detrimental to their substrates [2, 3], and cause biodeterioration of metal surfaces, known as biocorrosion [4, 5]. Biocorrosion is a severe problem to world maritime industries. Over 20% of all corrosions are associated biocorrosion, causing an estimated direct cost of 30 to 50 billion dollars annually [6, 7, 8]. The Duluth-Superior Harbor (DSH; Minnesota, USA), where we conducted this study, is severely affected by the problem of biocorrosion. The DSH is located on Lake Superior, the largest reservoir of freshwater in the world. In the DSH, about 20 kilometers of steel sheet piling appear to be affected, which may cost more than $200 million to replace [9]. In recent decades, the rate of corrosion in the Duluth-Superior Harbor appears to be more aggressive than previously observed [10]; the loss of steel in this harbor may be 2 to 12 times greater than in other similar freshwater environments [9, 11]. The aggressive rates of corrosion suggest there is some accelerating process acting on the steel, such as microbiologically influenced corrosion (MIC) [9, 11, 12]. Among the numerous organisms that colonize the surfaces of metals, sulfate-reducing bacteria (SRB) were previously associated to accelerated biocorrosion rates [12]. Corroding steel pilings in the Duluth-Superior harbor (DSH) have a rusty appearance characterized by orange, blister-like, raised tubercles on the surface [13]. These tubercles vary in diameter from a few millimeters to several centimeters and when removed, large and often deep pits (6 to 10 mm) are revealed in the steel, which is sometimes perforated. This pattern of corrosion is consistent with the appearance of corrosion caused by iron-oxidizing bacteria [14] and sulfate reducing bacteria [15] and similar to corrosion of steel structures recently observed at other harbors in Lake Superior.

Corrosion rates in the DSH vary with seasonal temperature changes, which is consistent with biological and chemical processes. Also, previous studies found that corroded steel surfaces and tubercles in the DSH, as well as in many other fresh water and sea water environment around the world, are covered by complex microbial biofilms that contain bacteria of the types responsible for corrosion of steel in other environments [3, 5, 16, 17, 18, 19]. Anoxic conditions created by microbial metabolism within these biofilms and corrosion tubercles are believed to be responsible for setting up electrical currents and copper precipitation in corrosion pits under the tubercles, both of which could accelerate the corrosion process [20].

Numerous strategies were previously used and developed to combat biocorrosion [21, 22, 23, 24]. In particular, biocidal compounds were widely used [21, 22]. However, their relatively low efficacy against biofilm, but most importantly their environmental hazard potential make these compounds unsatisfactory. To illustrate, tributyltin (TBT) was phased out 2008 due to their detrimental environmental effects, and despite its antifouling effectiveness [25]. Therefore, in order to address the combined ecological and economical requirements, efforts have focused on biological or benign molecules [23, 24]. Because bacterial biofilm formation has been associated to biocorrosion [3], molecules preventing the adhesion of bacteria or the formation of biofilms were tested in various coatings, and various substrates, including papers, polymers, glass and metals [21, 24, 26*]. Some compounds of biological origin, including antibiotics and/or bacteria producing antibiotics can impede the attachment of freshwater bacteria and prevent biofouling [27-33]. Additional studies have shown that when mild steel is protected by coatings of biofilm microbes that produce gramicidins, the steel corrosion rate is reduced 20 times compare to unprotected surfaces [31].

Another approach has recently emerged from the discovery and the understanding of bacterial communication. Indeed, biofilm production in bacteria, a key step in the biofouling process, can be regulated by Quorum Sensing (QS), a mechanism of chemical signaling used by numerous bacteria [34]. QS is the regulation of gene expression in response to fluctuations in cell density. QS bacteria produce and release into their environment chemical signal molecules, called autoinducers; a common class are acyl homoserine lactones (AHLs). Disruption of this bacterial communication has been shown to drastically reduce bacterial biofilms and virulence for numerous pathogens. A typical approach for disrupting QS consist of using AHL-degrading enzymes, dubbed lactonases. Through QS disruption, lactonases are capable of inhibiting bacterial virulence and bacterial biofilm formation, including in the context of biofouling [35, 36]. However, use of such enzymes to inhibit corrosion was not a possibility due to the inherent lack of environmental stability of proteins. It became possible with the recent identification and engineering of extremely stable lactonase variants that resist heat, denaturing agents, and organic solvents.

In this project, we took advantage of the existence of these enzymes, and have evaluated the anticorrosion activity of a variety of chemical (magnesium peroxide), biological (surfactin, capsaicin, and gramicidin), and enzymatic (a quorum quenching lactonase) bioactive coating additives in the context of the DSH water for a period of two months. We quantified corrosion by counting tubercules and examining SEM images of samples, and we determined the microbial community composition on the steel coated cross-linked silica gel containing the different additives (Table 9).

TABLE 9
List of antifouling biochemical compounds, enzyme and
bacteria tested in the experiment.

| Antifouling Treatments | Description |
|---|---|
| Surfactin | A very powerful surfactant commonly used as an antibiotic |
| | produced by Bacillus subtilis. It prevents biofilm attachment |
| | and formation by changing the hydrophilicity of the growth |
| | surface [27, 28]. |
| Magnesium peroxide | Reduces SRB abundance and sulfate reduction rate under |
| (MgO₂) | anoxic conditions by decreasing the sulfide concentration in the |
| | environment [29]. |
| Capsaicin | An active component of chili peppers. It is cytotoxic to biofilm- |
| | forming bacteria [30]. |
| Gramicidin (A, B, and C) | Antibiotic compounds obtained from the soil bacterial species |
| | Bacillus brevis. They are active against Gram-positive bacteria |
| | and select Gram-negative organisms |
| Bacillus brevis (ATCC | A Gram-positive, aerobic, spore-forming bacterium that |
| 9999) | produces gramicidins. It is commonly found in soil, air, and |
| | water [31]. |
| SsoPox W263I Lactonase | An improved variant of the hyperthermostable lactonase |
| | SsoPox from Sulfolobus solfataricus, exhibiting higher catalytic |
| | rates. This enzyme degrades acyl-homo N-acylhomoserine |
| | lactones (AHLs), the signaling molecules involved in bacterial |
| | quorum sensing [32, 33]. |

### Materials and Methods

### Origin and Production of the Bioactive Compounds

All biochemical (surfactin, MgO₂, capsaicin, Gramicidins) were purchased from Sigma Aldrich.

The production of Bacillus brevis (ATCC 9999) was performed by inoculating 10 ml of bacterial suspension (>1¹⁰/ml) into 500 ml of autoclaved medium containing 1.5 g beef extract and 2.5 g peptone (BD Difco, New Jersey, USA) for 24 hours at 37° C. with agitation at 100 rpm.

The SsoPox W263I production was performed as previously described [33]. Briefly, the production was carried out using the E. coli strain BL21(DE3)-pGro7/GroEL (Takara Bio). Cultures were performed in 500 mL of ZYP medium [37] (100 μg/ml ampicillin, 34 μg/ml chloramphenicol) as previously described [33], and 0.2% (w/v) arabinose (Sigma-Aldrich) was added to induce the expression of the chaperones GroEL/ES. Purification was performed as previously explained [33]. Briefly, a single heating step of 30 minutes incubation at 70° C. was performed, followed by differential ammonium sulfate precipitation, dialysis and exclusion size chromatography. Pure SsoPox W263I enzyme samples were quantified using a spectrophotometer (Synergy HTX, BioTek, USA) and a protein molar extinction coefficient as calculated by PROT-PARAM (Expasy Tool software) [38]. This purification protocol yields high purification grade enzyme (>95% purity) that can be used for crystallographic studies [39].

### Steel Coupons and Silica Gel Coatings

Steel coupons (5×2×0.95 cm) were cut from hot rolled ASTM-A328 steel, the same material used to construct steel sheet pilings used for most docks and bulkheads in most of the Duluth-Superior Harbor (DSH). The steel coupons were washed with soap water, lightly brushed for a few seconds with a test tube brush, and then rinsed with Milli-Q water to remove any loose material. Each coupon was designated with a unique number and weighed before being randomly assigned to a specific experimental treatment.

Currently, there are several bio-encapsulation and coating methods for applying antifouling bacteria or anti-corrosion biochemicals onto submerged steel surfaces (e.g. water tanks and ship hulls). Natural polymers are bio-compatible but lack mechanical strength and stability, while synthetic polymers are strong and stable but bio-compatibility is a problem [40]. Here, we used a silica gel coating matrix for the short-term testing of the antifouling biochemicals because it has great bio-compatibility, which is essential for antifouling agents to survive and perform. Prior research has demonstrated that synthetic silica coatings are effective for encapsulating biologically active materials. The bioactivity of biochemicals and enzymes can last for as long as several months, even after all cells are dead [40]. Silica gel coating also has the property of not being very durable, which allowed corrosion to occur in the time scale of this study.

The silica gel matrix (silicon alkoxide cross-linked silica nanoparticle gels), was made by a condensation process (polymerization) of TM40 silica nanoparticles and tetraethoxysilane (Sigma Aldrich Corp. St. Louis, Mo., USA).

Each compound or enzyme was added to 5 ml of the silica gel matrix to develop different coating treatments. The final concentrations are listed in Table 10. These coatings were applied by dipping coupons into the appropriate gel mixture for 1 minute, and then the coating on the coupon surface was air-dried at room temperature for 2 hours.

TABLE 10
Control and experimental treatments

| Coupon Set | Treatments |
|---|---|
| 1 | Bare Steel Control without coating |
| 2 | Control with agar coating |
| 3 | Control with crosslinked silica gel coating |
| 4 | Surfactin (50 μg/ml) in silica gel coating |
| 5 | MgO₂ (50 μg/ml) in silica gel coating |
| 6 | Capsaicin (20 μg/ml) in silica gel coating |
| 7 | Gramicidin (50 μg/ml) in silica gel coating |
| 8 | Bacillus brevis Migula (ATCC 9999) with agar coating |
| 9 | SsoPox lactonase enzyme (100 μg/ml) in silica gel |
| | coating |

Agar Coating was Used for Live Bacteria Bacillus brevis Encapsulation.

The Agar coating matrix was made by autoclave melting 4% agar into DI water and set in 50° C. water bath. 100 ml of Pre-inoculated bacteria culture of ATCC 9999 was added to the 100 ml of the agar matrix to develop the bacteria coating treatment. And 100 ml of the autoclaved medium was added to 100 ml of the agar matrix to develop the agar coating control. Coupons was dipped into the agar matrix then pulled out and cooled down to room temperature. Each coupon was covered in uniformed agar layer on all surfaces.

### Experimental Design and Sampling

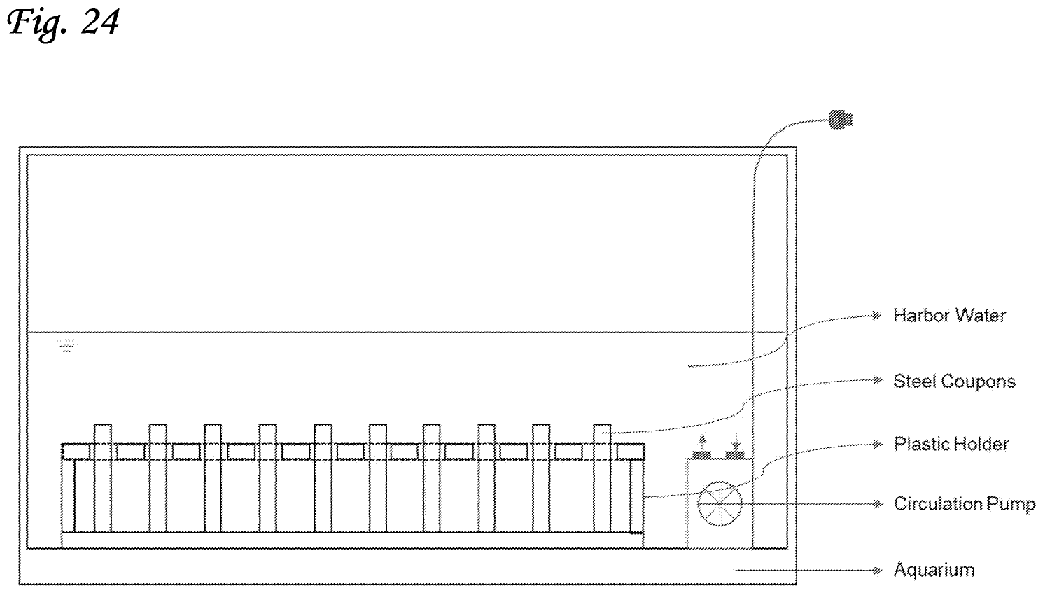

Six treatments and three controls were investigated for corrosion rate and changes in bacterial communities (Table 10). Each treatment or control contained three replicate steel coupons. After the triplicate steel coupons were coated with each biochemical, enzyme or bacterial treatment, they were incubated in experimental microcosms constructed from 10-gallon glass aquaria (Aqueon Glass, 50.8 cm×25.4 cm×30.5 cm) (FIG. 24). Each microcosm was equipped with an aquarium pump (Aquarium Systems Mini-Jet 404) to constantly circulate the water (˜2 L hr−1), and covered with a piece of acrylic with one corner cut out to allow gas exchange. The coupons were hold with plastic holders and immersed in water taken from the DSH for 8 weeks and then recovered for analyses.

### Corrosion and Microbial Analyses

The coupons in each treatment was photographed by the end of the experiment. Biocorrosion was evaluated by the number and coverage of corrosion tubercles, and also by imaging of coupon surfaces with an environmental scanning electron microscope (ESEM). The coupon images were captured with DSLR camera immediately after being removed from the harbor water, and then tubercle numbers and total area in digital images were measured using the analyze menu within ImageJ software (NIH, Bethesda, Md., USA). Surface roughness measurements were also made after the exposed coupons were cleaned with ASTM G1-90 iron and steel chemical cleaning procedure (2 min in 37% HCl, 50 g/L SnCl₂). A Hitachi TM-3030 ESEM was used to view details of the cleaned coupon surface and the 3D-View software was used to generate surface roughness measurements (SRa). Statistics analysis (T-tests) of the tubercle numbers, coverage and surface roughness were performed using Microsoft Excel software.

After photo imaging, on each of the coupon surface, all material including biofilm and tubercles were scraped into a sterile 50 ml polypropylene centrifuge tube (Corning, N.Y., USA) using a steel scraper. A 0.5 g subsample of the surface material from each coupon sample was used for DNA extraction using PowerSoil DNA kit (MoBio Laboratories). The extracted DNA was used to sequence the V4 region of 16S rDNA gene and describe changes in the composition of bacterial communities. DNA samples were quantified with a NanoDrop 2000 Spectrophotometer (Thermo Scientific, Waltham, Mass. USA) and then sent overnight to the University of Minnesota Genomics Center for 16S rDNA sequencing. 30 samples were multiplexed into a single run of Illumina MiSeq paired-end 300 cycles, which was expected to generate a total of 15 million sequences of the 254 bp portions of the 16S rDNA V4 region.

### Sequence Processing and Analysis

Sequence data were processed and analyzed using the MOTHUR program [41]. To ensure high quality data for analysis, sequence reads containing ambiguous bases, homopolymers >7 bp, more than one mismatch in the primer sequence, or an average per base quality score below 25 were removed. Sequences that only appear once in the total set were assumed to be a result of sequencing error and removed from the analysis. Chimeric sequences were also removed using the UCHIME algorithm within the MOTHUR program [42]. These sequences were clustered into operational taxonomic units (OTUs) at a cutoff value of ≥97%. Taxonomy was assigned to OTU consensus sequences by using the Ribosomal Database Project (RDP) taxonomic database. MOTHUR was also used to generate a Bray-Curtis dissimilarity matrix and calculate coverage. Bacterial and communities from different samples were compared using ANOSIM, a nonparametric procedure that tests for significant differences between groups, using Bray-Curtis distance matrices in mothur. Bacterial communities on tubercles of different treatments were compared using nonmetric multi-dimensional scaling ordinations in the program PC-ORD (MJM Software Designs, Gleneden Beach, Oreg.).

### Determination of the Dose-Response of Lactonase for Inhibiting Biocorrosion

Five treatments with lactonase enzyme and two controls of the steel coupons were investigated for enzyme activity and corrosion rate. Each treatment or control contained three replicate steel coupons. They were exposed in lake water in lab microcosm for a period of 7 weeks. Lactonase enzyme silica gel coated testing coupon are prepared as described before, except the coupons were dipped and dried twice and covered with 2 layers of the same coating to ensure the intactness of the coating. The enzyme concentrations used in this experiment were 100, 200, 500, 1000 μg/ml. Also 100 μg/ml equivalent activity lactonase raw extract was tested to compare the effectiveness of raw enzyme extract to the purified lactonase enzyme.

After 7 weeks of exposure, coupons were retrieved and analyzed. The coupons in each treatment was photographed by the end of the experiment. Biocorrosion was evaluated by quantities and coverage of corrosion tubercle. Surface roughness measurement were also made with procedure described above.

### Lactonase Enzyme Activity Test in Silica Gel Coating

In this experiment, we have tested the loss of enzyme activity in silica gel coating exposed in harbor water environment. Plastic applicators were coated with the lactonase treated silica gel coating for each treatment set. All coatings were made using the same method in the previous experiments. The enzyme concentrations used in this experiment were 100, 200, 500, 1000 μg/ml. The applicators were dipped into the fresh prepared coating with enzymes and then air dried in room temperature overnight before they were exposed in the same lake water microcosm with the testing steel coupons.

The enzyme activity was measured using Paraoxon enzyme activity assay using a previously described protocol [33]. Paraoxon enzyme activity assay were performed weekly with 3 of the enzyme coated applicators for each treatment. Applicators exposed in lake water were retrieved and briefly dried. Then each of the applicator was put into 5 mL 20 mM Paraoxon in PTE Buffer (50 mM Tris, 150 mM NaCl, 0.2 mM CoCl2) for 1 hr. Paraoxon hydrolysis was monitored by measuring absorbance at 412 nm with a spectrophotometer at the end of 1 hr reaction time. A standard of 8 ug/ml of lactonase enzyme was used in each test period.

### Lactonase Enzyme Activity Test in Acrylic Coating

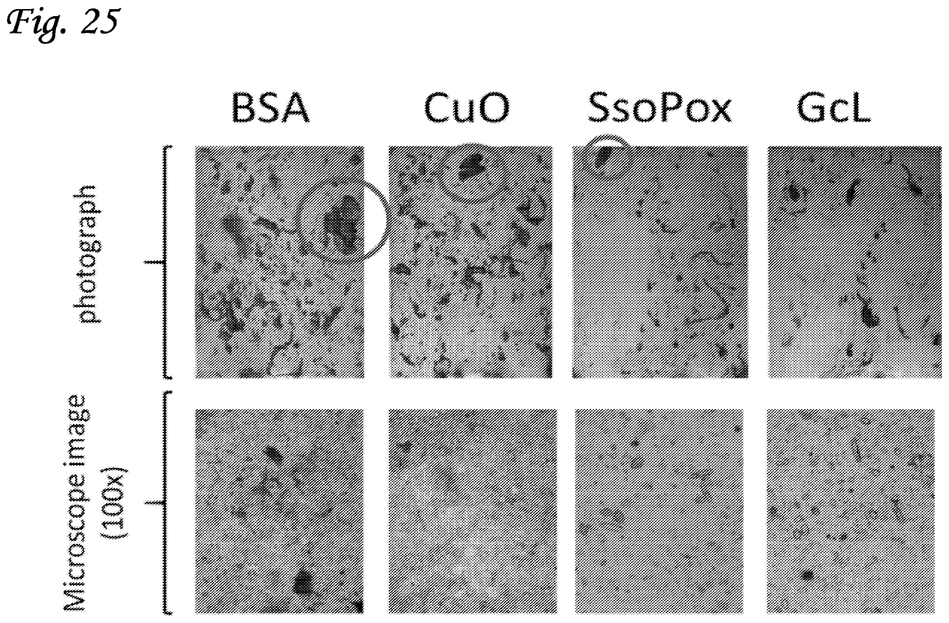

We showed during an experiment at lake Minnetonka (MN; Tonka Bay Marina) where coated polycarbonate sample coupons were submerged for 1 month that lactonase-containing acrylic base coating inhibits biofouling from algae, and from larger macroorganisms such as mussels (here Zebra mussels.). Comparison with controls (BSA, an inactive protein), copper oxide (a biocide, a widely active ingredient of antifouling coatings) show that at equal concentration (200 μg/mL), the lactonases SsoPox and GcL are more efficient at inhibiting biofouling than controls (FIG. 25). We note that microscopy imaging highlights large differences in the surface colonization between the different treatments.

### Results and Discussion

### Lactonase-Containing Coating Significantly Reduced Corrosion

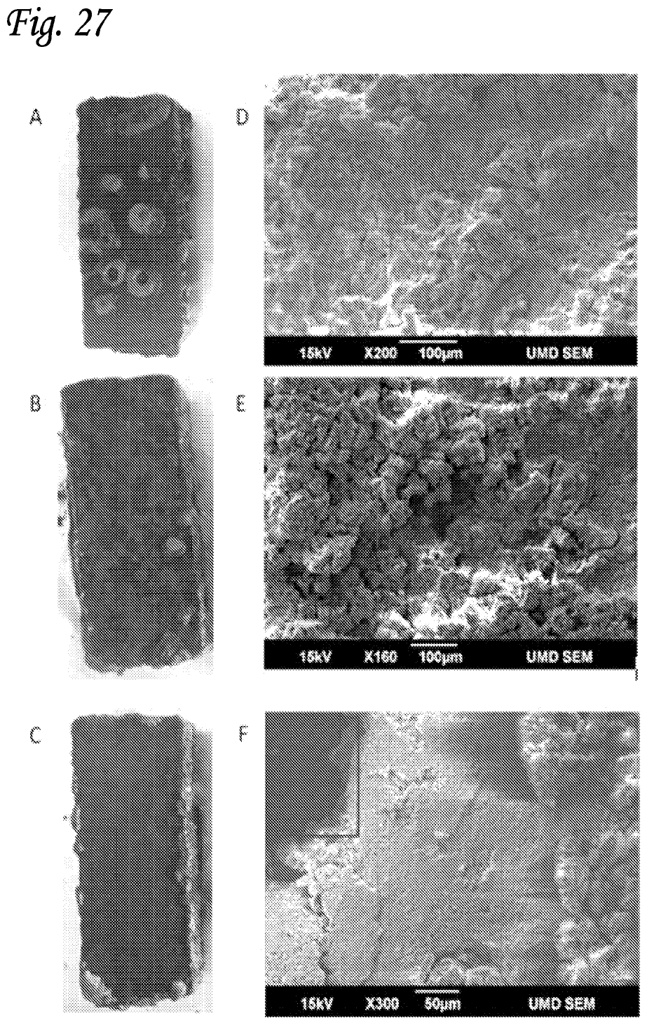

After 2 months of submersion of the DSH water, corrosion occurred on steel coupons: corrosion tubercles formed and grew on the steel surface, and the silica gel coating alone did not prevent the growth of the tubercle. While the use of a weak coating was desired to observed corrosion during the time course of the experimental setup, we note that the SEM analysis (FIG. 27F) suggests that the coating was peeling off by the end of the experiment for all treatments, and this may have limited the inhibition of corrosion

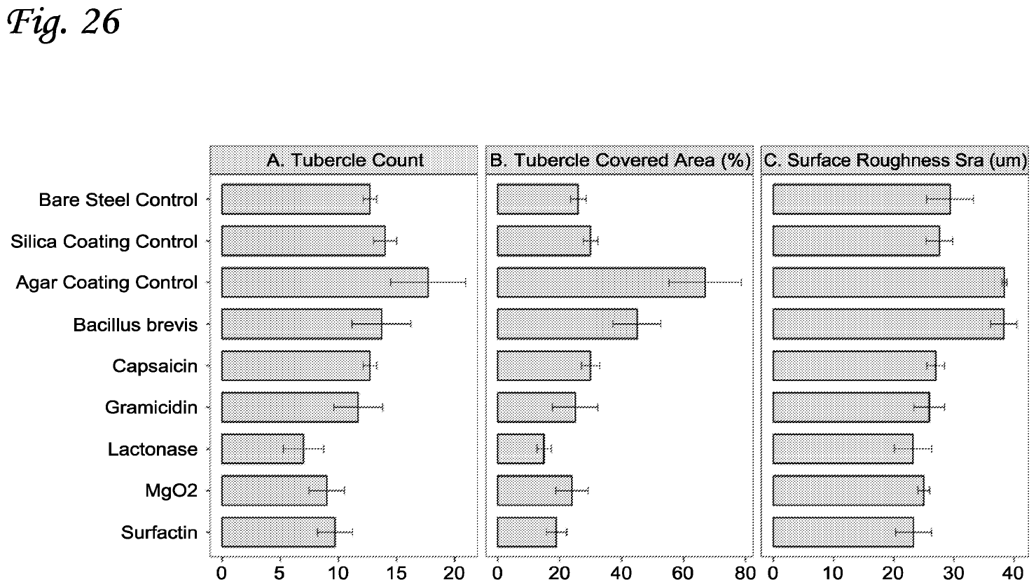

Our experiments using different treatments on the coated steel allowed us to observe reduction in corrosion, as illustrated by a reduction in number and percent coverage of corrosion tubercles as well as surface roughness (FIG. 26). However, the observed reduction are statistically significant only for two additives: surfactin on the one hand, and the lactonase enzyme on the other hand (FIG. 26, 27). Indeed, surfactin, a potent biocide, allows for a reduction of the number and the coverage of the tubercules (31% and 37%, respectively). In this study, the use of the lactonase as a coating additive yield to the strongest corrosion reduction, with the number and the coverage of tubercules being both reduced by 50%. We here note that the lactonase concentration used in this study was also higher than the concentrations of other tested chemicals, as enzyme may degrade and lose activity overtime. While the efficacy of surfactin on biofouling and biocorrosion was previously documented [27, 28], the ability of lactonase enzymes to inhibit corrosion was previously only envisioned [43, 44], but not demonstrated. In fact, quorum quenching approaches, and in particular using AHL lactonases were generally studied and focused on the QS systems of gram-negative bacteria [45]. The effect of QQ enzymes on more complex communities is uncertain. Yet, numerous reports demonstrate the ability of QQ enzymes, and particularly lactonases, to have a biological effect beyond isolated gram negative stains, including the inhibition of biofouling [46, 47]. Specifically, the ability of heterologously expressed lactonases to decrease fouling the context of membrane bioreactor was repeatedly demonstrated [35, 36, 47]. In this study, we demonstrated the ability of a purified, extremely stable lactonase, to inhibit biocorrosion over a period of 2 months.

All coating additives, including the lactonase enzyme, have changed the microbial community at the surface of the steel.

Microbial communities at the surface of the steel coupons was sampled and sequenced using Illumina MiSeq, which generated a total of 15,555,272 sequences for the 30 samples. After the sequence quality control procedures and chimera sequences removal, a data set of 7590591 sequences was extracted and used for bacterial taxonomy and community analysis. However, the three nucleic acids samples from the agar control was dropped from the Illumina sequencing because they to pass our stringent quality controls. The number of extracted sequences for each sample ranged from 123,203 to 492,370. To control for differences in number of sequence reads in each sample while still capturing as much of the diversity as possible, the number of sequences per sample was normalized by taking a randomly selected subsample of 123,203 sequences.

Nonmetric multidimensional scaling generated with the Illumina 16S rRNA sequences data indicated different bacterial communities developed on coupons in all treatments. Each treatment group has 3 data points representing microbial communities on triplicate experimental coupons. The NMDS showed the different treatment were well separated with the lowest stress value of 0.16 and an R-squared value of 0.89. Two of the experimental chemical treatments that reduced the formation of tubercles, and the coating only and bare steel control are circled in the NMDS plot. The changes of the bacterial communities are significant for all treatments, with p<0.05 comparing each treatment group to the coating control and bare steel group in ANOSIM test. Conversely, there is no statistical difference between the control with coating and the bare steel control, suggesting that the silica gel coating has no significant effect on the composition of bacterial community on the steel surface. While it was expected that the tested biocidal compounds would have an effect on bacterial populations at the surface of the steel, it is intriguing to note that the lactonase enzyme also alters the microbial composition of the surface. Lactonases, and the one used in this study in particular, are not biocides, and have no demonstrated effect of bacterial growth [48, 49]. The change in microbial communities induced by lactonases was also observed in a recent report on membrane bioreactor [50].

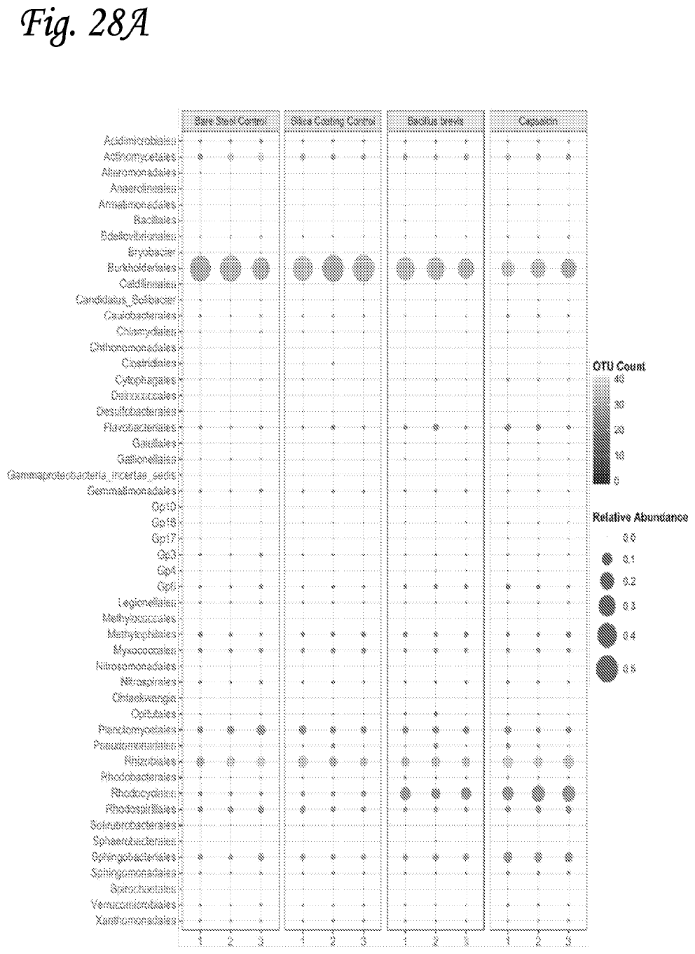

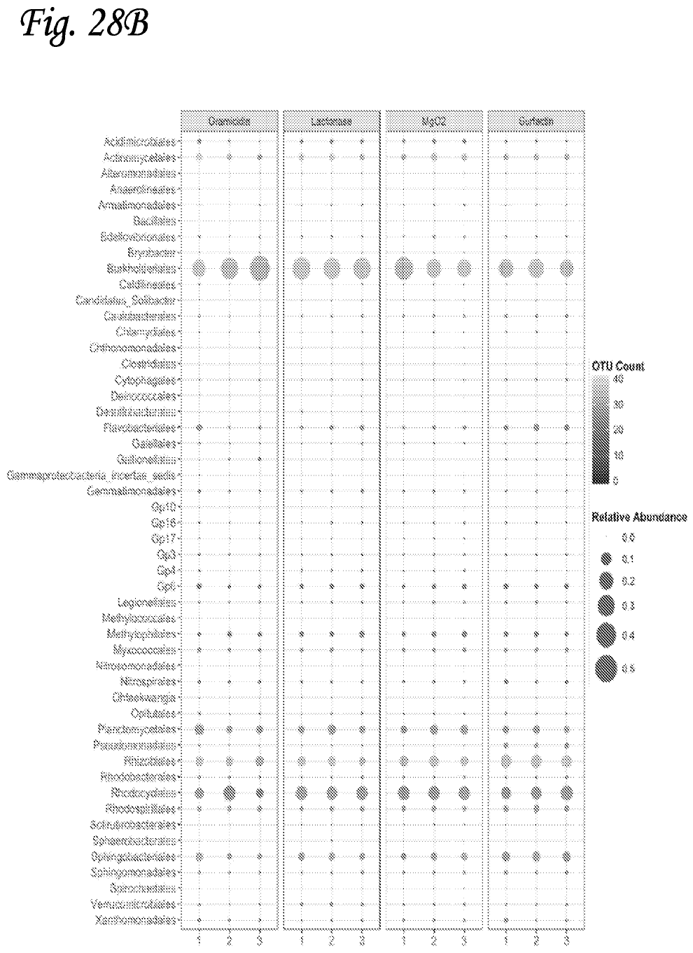

Order level taxonomy heatmap of the abundance and diversity of the top 50 bacteria across triplicate samples is shown in FIG. 28. Members of the Burkholderiales (30%), Rhodocyclales (8%) and Rhizobiales (8%) were the dominant bacteria found on all coupons. Certain orders of bacteria such as Burkholderiales, Pseudomonadales and Rhodospirillales were significantly reduced in both surfactin and lactonase treated samples. Burkholderiales is known to be able to oxidize iron, which has been reported to accelerate corrosion of iron [51, 52, 53]. And Rhodospirillales, Pseudomonadales are known to produce polysaccharides and accelerate biofilm formation [54]. The result suggests microbial were playing an important role in the process of corrosion and tubercle formation. And this also confirms that the effect of tubercle and corrosion reduction in the surfactin and lactonase treatment is caused by the change of bacterial community composition within surface biofilm and corrosion tubercles. Although orders of SRB such as Desulfobacterales, Desulfuromonadales and Desulfovibrionales were found in all samples, the sequence relative abundance were very low (<0.1%) comparing to iron oxidizers.

Increasing the Lactonase Concentration Did not Increase Protection from Corrosion.

In the screening experiment, we have found that the lactonase enzyme was the most effective coating additive to inhibit biocorrosion. Therefore, we varied the enzyme concentration to study the potential dose-response for this coating strategy. Additionally, we have compared the ability of highly pure, and raw extract containing enzyme to inhibit biocorrosion.

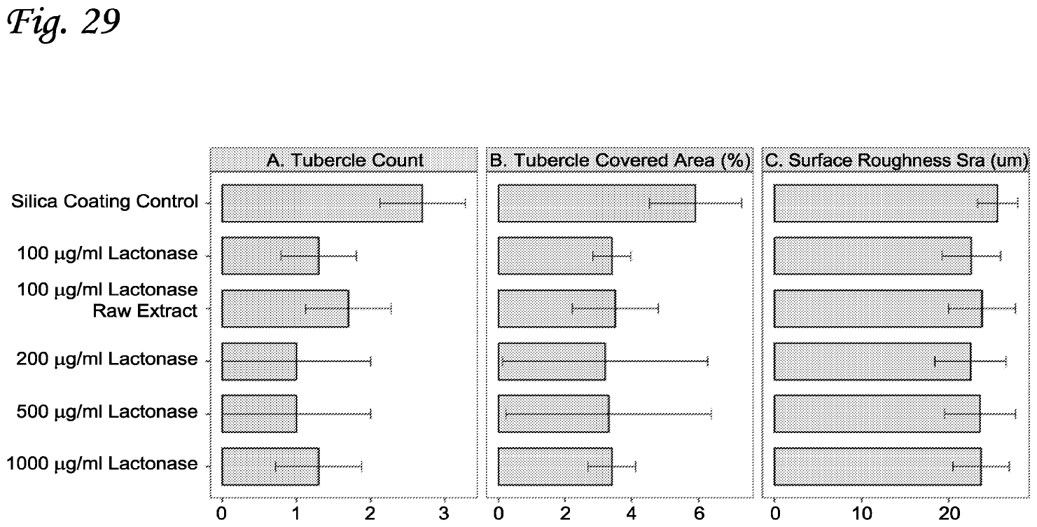

FIG. 29 shows result of tubercle count and coverage analysis. After 7 weeks of exposure in harbor water, the no coating control was heavily corroded and 45% of the surface area was covered with tubercles. The silica gel coating only control reduced the tubercle number and coverage significantly comparing to the no coating control, suggesting the when intact the silica gel coating may act as a protection layer preventing corrosion and microbial attachment. Images of retrieved experimental steel coupons with control silica gel coating and lactonase treated silica gel coating show significant differences in corrosion tubercle development after exposure in Duluth-Superior Harbor water. The All of the lactonase treatments of different concentrations were able to significantly reduce both number (>38%, p<0.05) and percent coverage (>41%, p<0.05) of corrosion tubercles on steel coupons. Reduction of surface roughness of all treatments were not statistically significant (FIG. 27). The highest reduction of corrosion tubercles was achieved in 200 μg/ml lactonase concentration. The effectiveness of raw extract was not significantly different than the purified enzyme (p=0.52). This result further demonstrated the lactonase treatments are able to reduce the formation of corrosion tubercles on steel surface, even at a low concentration of 100 μg/ml. The optimal concentration for coating application was determined at 200 μg/ml. The effectiveness of raw enzyme extract was not significantly different than the purified enzyme, while the production cost of such enzyme extract is greatly reduced comparing to the purified enzyme.

### Conclusions

Lactonase enzyme-containing coating showed the largest corrosion inhibition among the range of tested compounds in this study. Interestingly, the inhibition of corrosion did not increase with the increase of the enzyme dose in the coating. Additionally, this inhibition of biocorrosion is concomitant with a change in the composition of microbial communities at the surface of the steel. Different change is also observed for other tested molecules. However, while this change appears to be directly connected to the biocidal nature of the tested compounds, the induced change by the lactonase to the surface community probably derived from the properties of this enzyme, as it is not a biocide. Disruption of bacterial AHL-based quorum sensing may be the cause for the observed changes. These results demonstrate that coatings containing biological, non-toxic molecules are a potential alternative to biocide-containing coatings to prevent bio-induced corrosion.

### CITATIONS FOR EXAMPLE 4

1. O'Toole G, Kaplan H B, Kolter R. Biofilm formation as microbial development. Annual Reviews in Microbiology. 2000 October; 54(1):49-79.
2. Characklis W G. Bioengineering report: fouling biofilm development: a process analysis. Biotechnology and Bioengineering. 1981 Sep. 1; 23(9):1923-60.
3. Coetser S E, Cloete T E. Biofouling and biocorrosion in industrial water systems. Critical reviews in microbiology. 2005 Jan. 1; 31(4):213-32.
4. Videla H A, Characklis W G. Biofouling and microbially influenced corrosion. International Biodeterioration & Biodegradation. 1992 Jan. 1; 29(3-4):195-212.
5. Flemming H C. Biofouling in water systems-cases, causes and countermeasures. Applied microbiology and biotechnology. 2002 Sep. 1; 59(6):629-40.
6. Buck E, Maddux G C, Sullivan R L. Internal corrosion cost impact study: United States natural gas exploration and production industry. Topical report, June 1995-February 1996. Sullivan (RL) and Associates, Richardson, Tex. (United States); 1996 Feb. 1.
7. Dowling N J, Mittelman M W, White D C. The role of consortia in microbially influenced corrosion. Mixed Cultures in Biotechnology, McGraw Hill, New York. 1991:341-72.
8. Koch G H. HISTORIC CONGRESSIONAL STUDY: CORROSION COSTS AND PREVENTIVE STRATEGIES IN THE UNITED STATES. 2002.
9. 1. Marsh, C. P., J. Bushman, A. D. Beitelman, R. G. Buchheit, and B. J. Little. Freshwater Corrosion in the Duluth-Superior Harbor-Summary of Initial Workshop Findings. Special publication ERDC/CERL SR-05-3, U.S. Army Corps of Engineers; 2005.
10. Ray R, Lee J, Little B. Factors contributing to corrosion of steel pilings in Duluth-Superior Harbor. Corrosion. 2009 November; 65(11):707-17.
11. Oster, R. Modeling the Corrosive Loss of Port Infrastructure in the Duluth-Superior Harbor and the North Shore of Lake Superior. M.S. Thesis, University of Minnesota; 2012.
12. Bostrom, J. R. Microbiological and Chemical Aspects of Corrosion of Sheet Steel in the Duluth-Superior Harbor. M.S. Thesis, University of Minnesota; 2010.
13. Ray R I, Lee J S, Little B J, Gerke T L. The anatomy of tubercles: a corrosion study in a fresh water estuary. Materials and Corrosion. 2010 Dec. 1; 61(12):993-9.
14. Hamilton W A. Sulphate-reducing bacteria and anaerobic corrosion. Annual Reviews in Microbiology. 1985 October; 39(1):195-217.
15. Enning D, Venzlaff H, Garrelfs J, Dinh H T, Meyer V, Mayrhofer K, Hassel A W, Stratmann M, Widdel F. Marine sulfate—reducing bacteria cause serious corrosion of iron under electroconductive biogenic mineral crust. Environmental microbiology. 2012 Jul. 1; 14(7):1772-87.
16. Usher K M, Kaksonen A H, MacLeod ID. Marine rust tubercles harbour iron corroding archaea and sulphate reducing bacteria. Corrosion Science. 2014 Jun. 30; 83:189-97.
17. Sand W, Gehrke T. Microbially influenced corrosion of steel in aqueous environments. Reviews in Environmental Science and Biotechnology. 2003 Jun. 1; 2(2):169-76.
18. Vastra M, Salvin P, Roos C. MIC of carbon steel in Amazonian environment: Electrochemical, biological and surface analyses. International Biodeterioration & Biodegradation. 2016 Aug. 31; 1 12:98-107.
19. Cheung C S, Walsh F C, Campbell S A, Chao W T, Beech I B. Microbial contributions to the marine corrosion of steel piling. International biodeterioration & biodegradation. 1994 Jan. 1; 34(3-4):259-74.
20. Little B J, Mansfeld F B, Arps P J, Earthman J C. Microbiologically influenced corrosion. Wiley—VCH Verlag GmbH & Co. KGaA; 2007 Mar. 15.
21. Abdolahi A, Hamzah E, Ibrahim Z, Hashim S. Application of environmentally-friendly coatings toward inhibiting the microbially influenced corrosion (MIC) of steel: a review. Polymer Reviews. 2014 Oct. 2; 54(4):702-45.
22. Sadekuzzaman M, Yang S, Mizan M F, Ha S D. Current and recent advanced strategies for combating biofilms. Comprehensive Reviews in Food Science and Food Safety. 2015 Jul. 1; 14(4):491-509.
23. Scarascia G, Wang T, Hong P Y. Quorum Sensing and the Use of Quorum Quenchers as Natural Biocides to Inhibit Sulfate-Reducing Bacteria. Antibiotics. 2016 Dec. 15; 5(4):39.
24. Nir S, Reches M. Bio-inspired antifouling approaches: the quest towards non-toxic and non-biocidal materials. Current opinion in biotechnology. 2016 Jun. 30; 39:48-55.
25. Gipperth L. The legal design of the international and European Union ban on tributyltin antifouling paint: Direct and indirect effects. Journal of environmental management. 2009 Feb. 15; 90:S86-95.
26. Rani B E, Basu B B. Green inhibitors for corrosion protection of metals and alloys: an overview. International Journal of Corrosion. 2011 Sep. 26; 2012.
27. Vlamakis H, Chai Y, Beauregard P, Losick R, Kolter R. Sticking together: building a biofilm the Bacillus subtilis way. Nature Reviews Microbiology. 2013 Mar. 1; 11(3):157-68.
28. Korenblum E, de Araujo L V, Guimaraes C R, De Souza L M, Sassaki G, Abreu F, Nitschke M, Lins U, Freire D M, Barreto-Bergter E, Seldin L. Purification and characterization of a surfactin-like molecule produced by Bacillus sp. H2O-1 and its antagonistic effect against sulfate reducing bacteria. BMC microbiology. 2012 Nov. 7; 12(1):252.
29. Chang Y J, Chang Y T, Hung C H. The use of magnesium peroxide for the inhibition of sulfate-reducing bacteria under anoxic conditions. Journal of industrial microbiology & biotechnology. 2008 Nov. 1; 35(11):1481.
30. Xu Q, Barrios C A, Cutright T, Zhang Newby B M. Evaluation of toxicity of capsaicin and zosteric acid and their potential application as antifoulants. Environmental toxicology. 2005 Oct. 1; 20(5):467-74.
31. Zuo R, Wood T K. Inhibiting mild steel corrosion from sulfate-reducing and iron-oxidizing bacteria using gramicidin-S-producing biofilms. Applied microbiology and biotechnology. 2004 Nov. 1; 65(6):747-53.
32. Chow J Y, Xue B, Lee K H, Tung A, Wu L, Robinson R C, Yew W S. Directed evolution of a thermostable quorum-quenching lactonase from the amidohydrolase superfamily. Journal of Biological Chemistry. 2010 Dec. 24; 285(52):40911-20.
33. Hiblot J, Gotthard G, Chabriere E, Elias M. Structural and Enzymatic characterization of the lactonase Sis Lac from Sulfolobus islandicus. PLoS One. 2012 Oct. 10; 7(10):e47028.
34. Parsek M R, Greenberg E P. Sociomicrobiology: the connections between quorum sensing and biofilms. Trends in microbiology. 2005 Jan. 31; 13(1):27-33.
35. Oh H S, Yeon K M, Yang C S, Kim S R, Lee C H, Park S Y, Han J Y, Lee J K. Control of membrane biofouling in MBR for wastewater treatment by quorum quenching bacteria encapsulated in microporous membrane. Environmental science & technology. 2012 Apr. 11; 46(9):4877-84.
36. Lade H, Paul D, Kweon J H. N-Acyl homoserine lactone-mediated quorum sensing with special reference to use of quorum quenching bacteria in membrane biofouling control. BioMed research international. 2014 Jul. 24; 2014.
37. Studier F W. Protein production by auto-induction in high-density shaking cultures. Protein expression and purification. 2005 May 31; 41(1):207-34.
38. Gasteiger E, Hoogland C, Gattiker A, Duvaud S E, Wilkins M R, Appel R D, Bairoch A. Protein identification and analysis tools on the ExPASy server. Humana Press; 2005.
39. Hiblot J, Gotthard G, Elias M, Chabriere E. Differential active site loop conformations mediate promiscuous activities in the lactonase SsoPox. PLoS One. 2013 Sep. 23; 8(9):e75272.
40. Mutlu B R, Yeom S, Tong H W, Wackett L P, Aksan A. Silicon alkoxide cross-linked silica nanoparticle gels for encapsulation of bacterial biocatalysts. Journal of Materials Chemistry A. 2013; 1(36):11051-60.
41. Schloss P D, Westcott S L, Ryabin T, Hall J R, Hartmann M, Hollister E B, Lesniewski R A, Oakley B B, Parks D H, Robinson C J, Sahl J W. Introducing mothur: open-source, platform-independent, community-supported software for describing and comparing microbial communities. Applied and environmental microbiology. 2009 Dec. 1; 75(23):7537-41.
42. Edgar R C, Haas B J, Clemente J C, Quince C, Knight R. UCHIME improves sensitivity and speed of chimera detection. Bioinformatics. 2011 Aug. 15; 27(16):2194-200.
43. Defoirdt T, Boon N, Bossier P. Can bacteria evolve resistance to quorum sensing disruption?. PLoS pathogens. 2010 Jul. 8; 6(7):e1000989.
44. Scarascia G, Wang T, Hong P Y. Quorum Sensing and the Use of Quorum Quenchers as Natural Biocides to Inhibit Sulfate-Reducing Bacteria. Antibiotics. 2016 Dec. 15; 5(4):39.
45. Fetzner S. Quorum quenching enzymes. Journal of biotechnology. 2015 May 10; 201:2-14.
46. Gül B Y, Koyuncu I. Assessment of New Environmental Quorum Quenching Bacteria as a Solution for Membrane Biofouling. Process Biochemistry. 2017 Jun. 10.
47. Siddiqui M F, Rzechowicz M, Harvey W, Zularisam A W, Anthony G F. Quorum sensing based membrane biofouling control for water treatment: A review. Journal of Water Process Engineering. 2015 Sep. 30; 7:112-22.
48. Hraiech S, Hiblot J, Lafleur J, Lepidi H, Papazian L, Rolain J M, Raoult D, Elias M, Silby M W, Bzdrenga J, Bregeon F. Inhaled lactonase reduces Pseudomonas aeruginosa quorum sensing and mortality in rat pneumonia. PloS one. 2014 Oct. 28; 9(10):e107125.
49. Guendouze A, Plener L, Bzdrenga J, Jacquet P, Rémy B, Elias M, Lavigne J P, Daudé D, Chabrière E. Effect of Quorum Quenching Lactonase in Clinical Isolates of Pseudomonas aeruginosa and Comparison with Quorum Sensing Inhibitors. Frontiers in microbiology. 2017; 8.
50. Jo S J, Kwon H, Jeong S Y, Lee S H, Oh H S, Yi T, Lee C H, Kim T G. Effects of Quorum Quenching on the Microbial Community of Biofilm in an Anoxic/Oxic M BR for Wastewater Treatment. Journal of microbiology and biotechnology. 2016 Sep. 1; 26(9):1593-604.
51. Hedrich S, Schlömann M, Johnson D B. The iron-oxidizing proteobacteria. Microbiology. 2011 Jun. 1; 157(6):1551-64.
52. Liu F, Zhang J, Zhang S, Li W, Duan J, Hou B. Effect of sulphate reducing bacteria on corrosion of Al Zn In Sn sacrificial anodes in marine sediment. Materials and Corrosion. 2012 May 1; 63(5):431-7.
53. Quaiser A, Bodi X, Dufresne A, Naquin D, Francez A J, Dheilly A, Coudouel S, Pedrot M, Vandenkoornhuyse P. Unraveling the stratification of an iron-oxidizing microbial mat by metatranscriptomics. PLoS One. 2014 Jul. 17; 9(7):e102561.
54. Garrity G, Staley J T, Boone D R, De Vos P, Goodfellow M, Rainey F A, Schleifer K H. Bergey's Manual of Systematic Bacteriology: Volume Two: The Proteobacteria. Brenner D J, Krieg N R, editors. Springer Science & Business Media; 2006 Jul. 25.

### Example 5

### Lactonases Protect Plants from Infection

Numerous bacterial pathogens infect crop plants, representing major economic burdens, and limit our ability to feed the world's populations. Current methods for controlling plant diseases due to bacterial infection have had limited success, in part due to bacterial resistance and specificity. Novel strategies are therefore greatly needed to control microbes. Numerous bacterial pathogens use chemical signaling systems to coordinate virulence factor expression and biofilm formation. A common bacterial communication mechanism called quorum sensing (QS) regulates bacterial gene expression in response to fluctuations in cell density. A common class of QS molecules are acyl homoserine lactones (AHLs). The hydrolysis of AHLs lead to the disruption of bacterial communication, and a subsequent reduction of biofilm formation and virulence. The use of a controlled biologically-derived agent, e.g. a lactonase preparation, to control plant pathogens, is therefore appealing. Our group has isolated and engineered enzymes that are highly proficient and extremely stable, that can be used as biocontrol agents and be active at all times, independently of the ecosystem. Over the last year, we have demonstrated that this approach can protect a variety of plants, including corn, from infection. Our results were exciting as we learned that crop protection is broad, extending from grasses (Corn, Wheat, and Barley) to Dicots (Soybean, Field Beans, and Potato).

### BACKGROUND

Many bacterial pathogens infect crop plants causing huge economic losses that also limit our ability to feed the world's populations. Current methods for controlling plant diseases due to bacterial infection, mostly through the use of chemical pesticides, have had limited success, in part due to bacterial resistance, specificity, and environmental, regulatory, and policy repercussions due to pesticide use. Therefore, novel strategies are currently needed to control microbes infecting plants.

Numerous bacterial pathogens use chemical signaling systems to coordinate expression of their virulence factors. These are the same gene systems involved in biofilm formation. A common bacterial communication mechanism called quorum sensing (QS) regulates bacterial gene expression in response to fluctuations in cell density. The QS bacteria produce and release into their environment chemical signal molecules, called autoinducers. A common class of autoinducer-QS molecules are acyl homoserine lactones (AHLs). AHL-mediated communication is critical for expression of bacterial virulence factors and is present in most gram negative and some gram-positive bacterial pathogens of a wide variety of plants. Disruption of AHL communication via quorum quenching (QQ) enzymes (lactonases) control pathogens by reducing virulence and has been shown on cell cultures and in vivo.

The ability of lactonase enzymes to control bacterial virulence is extremely appealing for crop protection. The first identified lactonase from Bacillus thuringensis, AiiA, was used to produce genetically modified plants. The expression of AiiA in tobacco and potato plants significantly reduced maceration area of leaves (tobacco) or tubers (potato), upon infection with Pectobacterium carotovorum i. In addition to ectopic expression of lactonases in plants, a relatively new emerging quorum quenching technique is the use of bacteria, which naturally employ quorum quenching enzymes as biocontrol agents to manipulate QS pathways. Several studies have demonstrated effective biocontrol activity through the application of bacteria harboring AHL-degrading enzymes to infected plants². However, and to the best of our knowledge, the efficiency of such biocontrol agents has not yet been demonstrated in the field. This is in large part due to: 1) the lack of ability of these agents to adequately express enzymes under field conditions, 2) the fact that enzymes are susceptible to degradation in the environment, 3) QQ bacterial strains do not exhibit rhizosphere competence, and root and shoot colonization ability, or 4) the inability to survive, proliferate and produce enzymes on growing plant roots and leaves in the presence of indigenous microbial population³.

The use of a more controlled biologically-derived disease control agent, e.g. a lactonase preparation, is therefore appealing. Importantly, the enzyme(s) would be present and active at all times, independently of the ecosystem. However, most enzymes are very unstable under environmental conditions, mainly due to bacterial-produced proteases and unfavorable physical conditions, e.g. pH or water activity.

To overcome these problems, we have isolated a lactonase from an extremophile, and engineered it to be extremely stable. The lactonase SsoPox, isolated from the hyperthermophilic bacterium Sulfolobus solfataricus, exhibits a melting temperature of 106° C.^{4,5}. We have further engineered Ssopox to increase its lactonase catalytic activity⁶, and it is stable towards aging (several years), detergents, pH, chemicals, organic solvents, proteases, and disinfection methods⁵⁻⁷. These properties make Ssopox a good candidate for scale-up of the protein production and use in the environment. In these studies, we used SsoPox-W263I variant.

Protection of Potato Tubers, Wheat, Barley and Corn Plants from Bacterial Infections.

Current lab production yields are typically 1 g of pure (>95% purity) compound for every 3 L of culture, and numerous applications could use partially purified enzyme preparations. The SsoPox QQ enzyme therefore represent a unique candidate to control pathogens and protect plants and crop from bacterial infections.

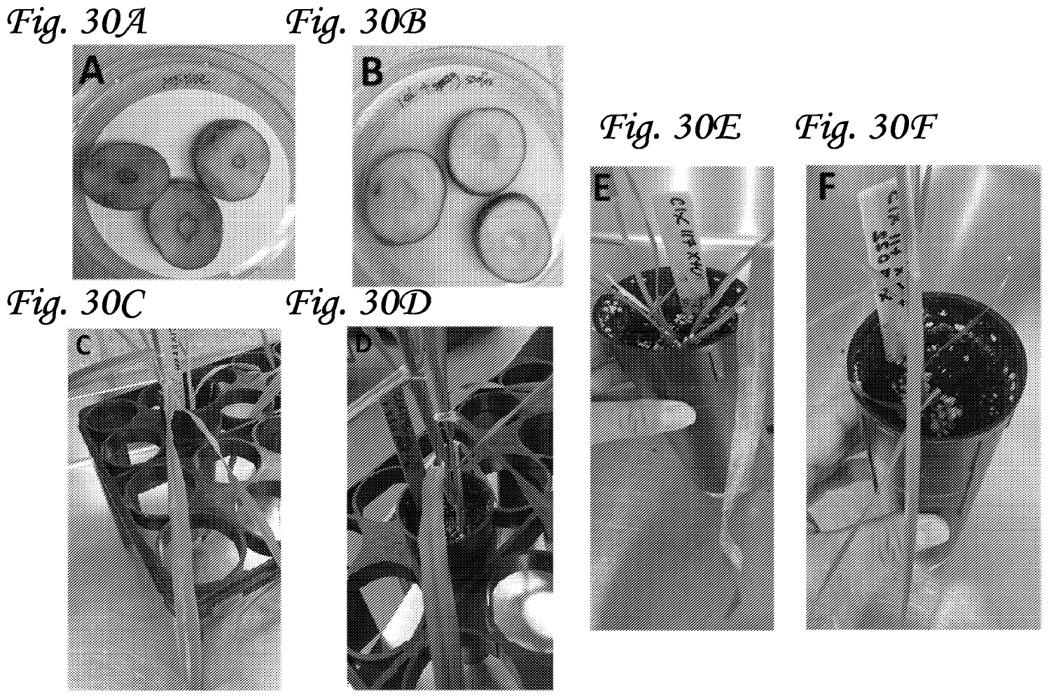

Specifically, we could establish infection systems for corn, wheat, and barley plants, and a crop infection system for potato (tubers and leaves). In all of these systems, we demonstrated that the treatment with a lactonase, consisting of a single spraying of the surface of the leaves with a small volume of enzyme (10 mg/mL) was sufficient to protect corn plants from the tested pathogens, including when inoculation was performed with a large numbers of cells (FIG. 30).

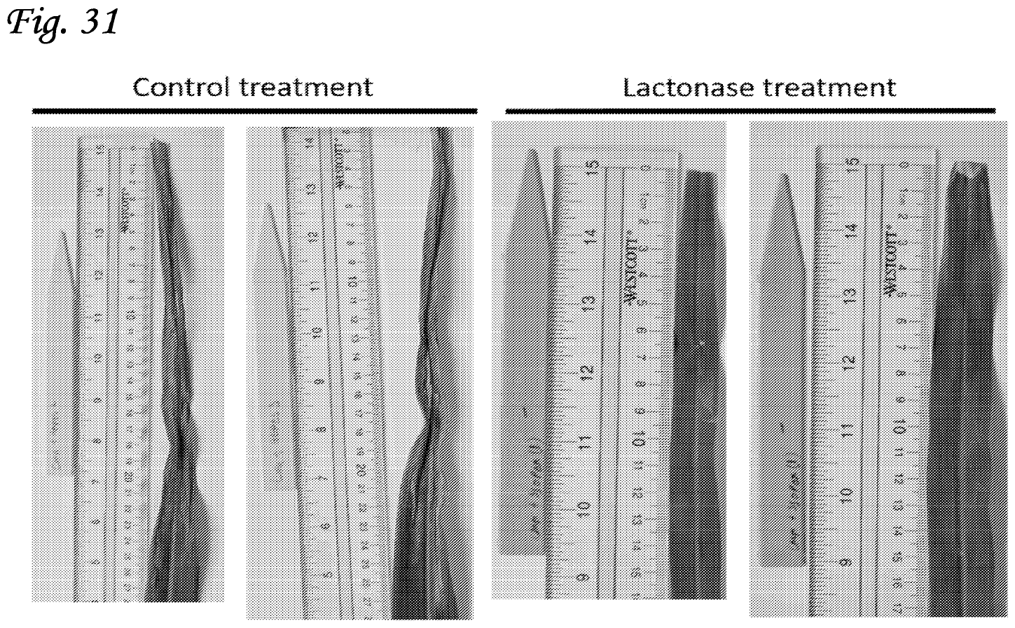

Moreover, we specifically established a corn infection assay using the pathogen Clavibacter michiganensis subsp. Nebraskensis. After treatment with the lactonase spray, we report here that it protected the plant from showing any symptoms of infection (FIG. 31).

Protection of Kidney Beans (Whole Plant) from Pseudomonas syringae pv Syringae Infection.

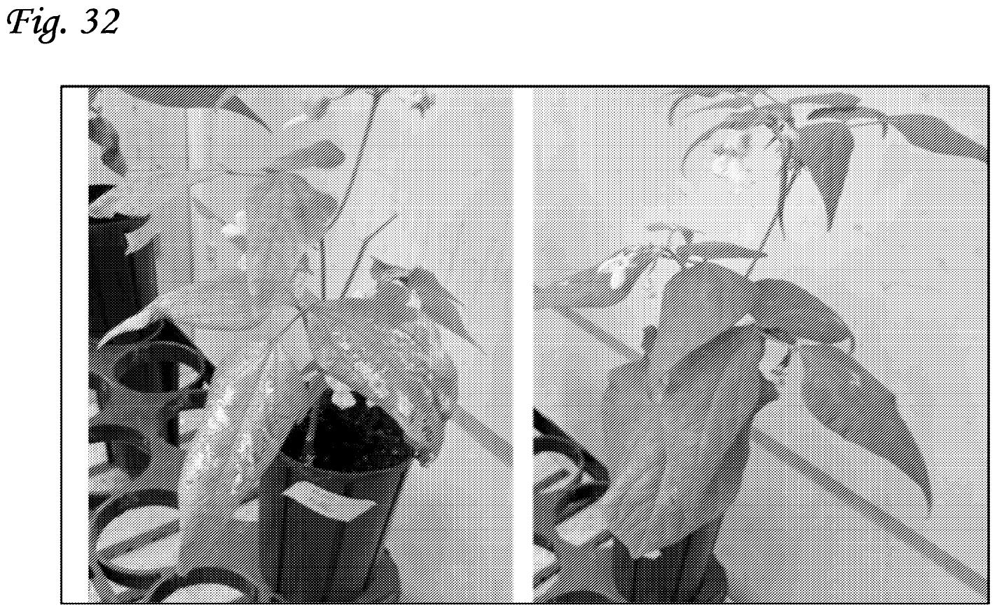

Kidney bean plants were grown and infected with P. syringae pv. Phaseolicola. One plant was sprayed with a lactonase solution and this protected the plant from infection by multiple plant pathogens inoculations for the duration of the experiment (14 days) (FIG. 32).

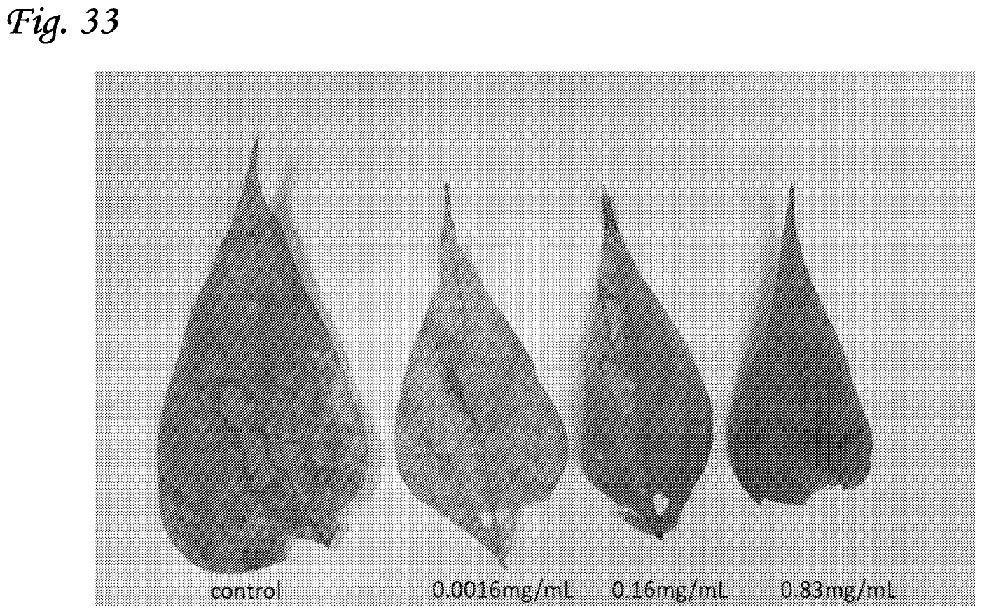

Additionally, we performed a dose-dependent experiment on this plant infection model by varying the concentration of the sprayed enzyme (FIG. 33).

We believe that these results, obtained by simply spraying a 100% natural, biodegradable, ecological, non-toxic, and biological molecule on plant leaves are spectacular. Based on these results, we are extremely confident and enthusiastic about the potential of lactonases to be a leading compound for the industry. We believe that these results call for a more comprehensive assessment of the capacities of this molecule to be used for plant and crop protection under field conditions.

### CITATIONS FOR EXAMPLE 5

1 Dong, Y.-H. et al. Quenching quorum-sensing-dependent bacterial infection by an N-acyl homoserine lactonase. Nature 411, 813-817 (2001).
2 Cirou, A. et al. Gamma-caprolactone stimulates growth of quorum-quenching Rhodococcus populations in a large-scale hydroponic system for culturing Solanum tuberosum. Research in microbiology 162, 945-950 (2011).
3 Benizri, E., Baudoin, E. & Guckert, A. Root colonization by inoculated plant growth-promoting rhizobacteria. Biocontrol science and technology 11, 557-574 (2001).
4 Merone, L., Mandrich, L., Rossi, M. & Manco, G. A thermostable phosphotriesterase from the archaeon Sulfolobus solfataricus: cloning, overexpression and properties. Extremophiles:life under extreme conditions 9, 297-305, doi:10.1007/s00792-005-0445-4 (2005).
5 Hiblot, J., Gotthard, G., Chabriere, E. & Elias, M. Characterisation of the organophosphate hydrolase catalytic activity of SsoPox. Sci. Rep. 2, 779, (2012) https://doi.org/10.1038/srep00779.
6 Hiblot, J., Gotthard, G., Elias, M. & Chabriere, E. Differential active site loop conformations mediate promiscuous activities in the lactonase SsoPox. PLoS One 8, e75272, doi:10.1371/journal.pone.0075272 (2013).
7 Remy, B. et al. Harnessing hyperthermostable lactonase from Sulfolobus solfataricus for biotechnological applications. Scientific Reports 6 (2016).

### Example 6

### Lactonases Protect from Bacterial Infection

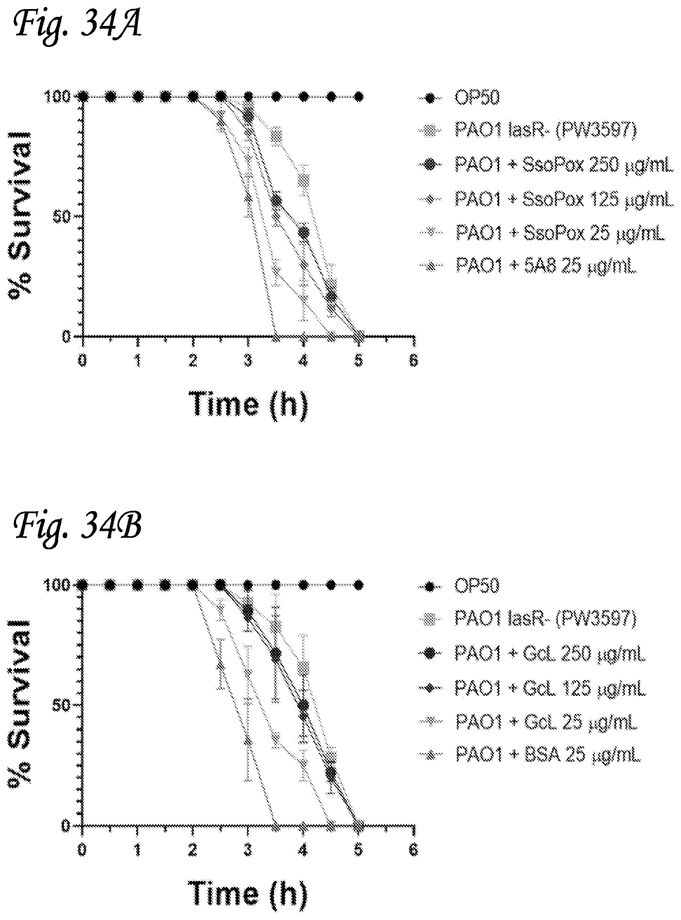

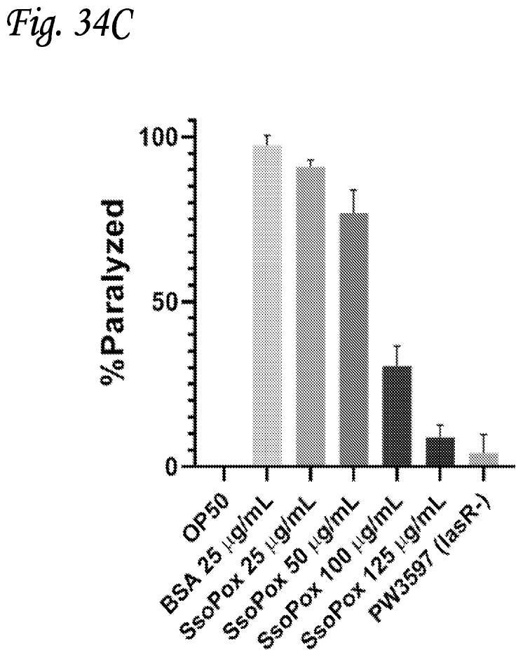

We show that treatment with lactonases GcL and SsoPox can increase survival in two different infection models of Caenorhabditis elegans by Pseudomonas aeruginosa. The protection by lactonases is dose-dependent (FIG. 34) in both models.

### Example 7

### Lactonases Change Microbial Population Compositions

### Abstract

The disruption of bacterial signaling (quorum quenching) has been proven to be an innovative approach to affect the behavior of bacteria. In particular, lactonase enzymes that are capable of hydrolyzing the N-acyl homoserine lactone (AHL) molecules used by numerous bacteria, were reported to inhibit biofilm formation, including those of freshwater microbial communities. However, insights and tools are currently lacking to characterize, understand and explain the effects of signal disruption on complex microbial communities. Here we created silica capsules containing an engineered lactonase that exhibits quorum quenching activity. Capsules were used to design a filtration cartridge to selectively degrade AHLs from a recirculating bioreactor. The growth of a complex soil microbial community in the bioreactor was monitored in the presence and in the absence of lactonase over a 3 week period. Data reveals that a lactonase-embedded filtration cartridge can effectively reduce biofilm formation in a water recirculating system, and that biofilm inhibition is concomitant to a drastic change in the composition of the communities within these biofilms. Changes in microbial composition relates to the relative proportion of genera, but also the specific presence or absence of some genera depending upon the use of the lactonase enzyme. Additionally, we demonstrate that AHLs signal disruption induce a dramatic composition change in a soil community. This unexpected finding is evidence for the importance of signaling in the competition between bacteria within communities. This study provides foundational tools and data for the investigation of the importance of AHLs-based signaling in complex community contexts.

### INTRODUCTION

Bacterial quorum sensing (QS) is one of the most prominent and studied communication systems used by bacteria¹. Numerous bacteria produce and utilize chemical signal molecules to coordinate, in a cell density dependent manner, their behaviors^{2,3}. Bacterial quorum sensing was shown to regulate various behaviors in numerous microbes, including virulence and biofilm formation³. Biofilms are slimy layers of a hydrated matrix of polysaccharides, proteins and nucleic acids produced by bacteria and can attach to surfaces⁴. These structured communities enable a multicellular existence that is distinct from the planktonic state⁵.

Some enzymes, named quorum quenching (QQ) enzymes, are naturally capable of interfering with this QS via the enzymatic degradation of autoinducer molecules^{3,6}. This was particularly studied in the case of the autoinducer-1, N-acyl homoserine lactones (AHLs)⁷⁻⁹. Indeed, the disruption of bacterial signaling using QQ enzymes was previously shown to inhibit the production of virulence factors and the biofilm production of numerous pathogens, both in vitro¹⁰⁻¹⁴ and in vivo^{12,13}. These properties are making QQ enzymes prime candidates for bacterial control in numerous fields of application, yet efforts are required to overcome their drawbacks such as activity levels, activity at low or high temperatures, stability, and production costs^{15,16}.

A promising enzyme candidate to overcome the intrinsic limitations listed above, is the lactonase, SsoPox, isolated from the hyperthermophilic crenarcheon, Sulfolobus solfataricus¹⁷⁻¹⁹. This enzyme belongs to the Phosphotriesterase-Like Lactonase family^{20,21}, and is naturally hydrolyzing a wide range of AHLs, from C6 AHL to 3-oxo C12 AHL²². SsoPox was shown to disrupt bacterial quorum sensing in vitro, as well as in vivo^{13,14}. Additionally, this lactonase was reported to be catalytically active over a very wide range of temperatures, from −19° C. to 70° C.^{16,17} Interestingly, this lactonase exhibits exceptional thermal stability (Tₘ=106° C.²³), resistance to denaturing agents, organic solvents, detergents, radiations, bacterial secretions and proteases^{16,23}. The resolution of the crystal structure of SsoPox revealed the critical importance of residue W263, interacting with the bound lactone ring of the AHL molecule^{19,24}. Mutation of this residue allowed for generation of variants with higher lactonase catalytic activity, such as W263I^{22,25}.

While the substrate specificity of several lactonases has been determined^{22, 26-32}, the range of bacteria that can be controlled by these enzymes is unclear. Indeed, AHL-based quorum sensing and effects of quorum sensing interference were mostly described in gram negative bacteria^{10, 13, 14, 33, 34} yet studies report activity of lactonase of bacterial strains that are not known for using AHLs^{33,35}. Moreover, lactonases were reported to inhibit biofilm formation in complex communities, particularly in the context of biofouling^{8, 9, 36}. The presence of bacteria expressing lactonases was shown to reduce biofouling in a membrane bioreactor (MBR)^{8, 9, 36}, and affect the microbial community attached to the membrane³⁷. Tools and insights are missing to adequately document these effects and decipher the mechanisms underlining these observations.

In order to determine the effects of AHL degradation in the context of a complex soil microbial community, we used a silica gel bioencapsulation technique. Silica is a cytocompatible material in which bacteria and enzymes are physically confined, retained within the matrix and protected from the environment³⁸⁻⁴³. Here, encapsulated E. coli cells overexpressing the lactonase SsoPox W263I were used to produce beads. Encapsulation of bacteria overexpressing stable, engineered lactonases combines the intrinsic properties of the SsoPox enzyme, the lower production costs associated to the use of cells instead of purified enzyme, and a robust, permeable silica structure facilitating the integration of this enzyme in water treatment systems.

Catalytically active capsules were used as an enzymatic filtration matrix to degrade AHL signaling molecules produced by a complex soil microbial community cultured in a recirculating system. We determined that the presence of the lactonase in the filtration beads leads to a dramatic (2-fold) reduction of biofilm formation over the course of the experiment (21 days), and that this reduction is associated with a change of the microbial population forming the biofilm. This experimental system opens up a new way to study the importance of bacterial signaling, the effects of signal disruption using lactonases and highlights the potential of these enzymes to serve in a water treatment, including recirculating, system.

### Materials and Methods

### Preparation of Silica Lactonase-Containing Beads

The Quorum Quenching (QQ) lactonase (SsoPox W263I) and control protein (inactive mutant SsoPox 5A8; carrying the mutations V27G/P67Q/L72C/Y97S/Y99A/T177D/R223 L/L226Q/L228M/W263H, obtained previously²²), were overexpressed in E. coli BL21-pGro7 (Grown to OD_{600 nm}=0.8 at 37° C., 200 RPM shaking) as previously described^{22, 23, 25}. After overnight induction (18° C., 0.2% L-Arabinose, 200 RPM shaking), cells overexpressing proteins were centrifuged (4,400×g, 20 min, 4° C.) and re-suspended in 100 mM potassium phosphate buffer, pH 7, at a concentration of 0.4 g/mL wet weight (0.2 g/mL for the 1× lactonase beads). Gel beads (1 mm diameter) containing the lactonase/control bacteria cultures were made using a dripping method while gelation occurred, using a method similar to a previously used protocol⁴¹ 400 mg PEG (average molecular weight, 10,000 Da) was mixed with 4 mL acetic acid (0.01M) until the PEG dissolved. 2.5 mL TMOS (Tetramethyl orthosilicate, 98%) was then added and allowed to stir for 30 minutes until the solution became clear. 1 mL of cell suspension (0.2 g/mL) was mixed with the PEG/TMOS/acetic acid solution and gelation occurred within a few minutes. The bacteria-encapsulated beads (8 mL) were added directly to empty chromatography columns to create filtration cartridges. A filter at the outlet of the column ensured the amount of beads present in the column would be constant throughout the duration of the experiment. We therefore produced two different types of silica beads: (i) beads where E. coli cells overexpressing the lactonase SsoPox W263I are entrapped, dubbed lactonase beads, and (ii) beads where E. coli cells overexpressing a control protein (inactive mutant 5A8) are entrapped, dubbed control beads. These beads were used to produce three distinct filtration cartridges: (a) the 2× lactonase cartridge, containing only (total of 8 mL) lactonase beads, (b) the control cartridge containing only control beads (total of 8 mL) and (c) the 1× lactonase cartridge containing a 1:1 ratio (4 mL+4 mL, total of 8 mL) of lactonase beads and control beads.

### Kinetic Assays of Lactonase-Containing Gels

Using the same dripping method described above, lactonase-containing gel and gel containing the control protein were poured into 96 well plates for quantification of the enzyme activity over extended periods of time (28 weeks). Each well contained 75 μL of gel and was stored at 4° C. in the presence of the pte buffer (50 mM HEPES, pH 8, 150 mM NaCl, 0.2 mM COCl2) or the lactonase buffer (2.5 mM Bicine pH 8.3, 150 mM NaCl, 0.2 mM CoCl2, 0.2 mM cresol purple, 0.5% DMSO). The high level of transparency of the gel allowed for the use of a microplate reader (Synergy HT, BioTek, USA; Gen5.1 software) to measure kinetics. The gel volume plus the buffer volume was equal to 200 μL (6.2 mm path length). Before testing activity, plates were allowed a few minutes to equilibrate to room temperature. For the lactonase assay, kinetics were performed as previously described^{28, 29, 31, 32}. Lactonase activity is expressed in enzymatic units defined as M of substrate hydrolyzed per min per mg of cells (wet weight). All kinetic measurements were performed as triplicates. For both lactonase and phosphotriesterase activities, activities of control gels (containing E. coli cells overexpressing mutant 5A8) were subtracted to the measured activities of the lactonase gels (containing E. coli cells overexpressing SsoPox W263I).

In order to evaluate the durability of gels over time, we used the chromogenic substrate paraoxon as a proxy for the enzyme activity, using a previously described assay^{23, 25, 44}. Assays were performed using 10 μL of 20 mM paraoxon (1 mM final concentration) to reach a final reaction volume of 200 μL. The paraoxon degradation product (paranitrophenolate) could be directly measured in a spectrophotometer at 405 nm (F=17000 M⁻¹ ·cm⁻¹). Activity over time was normalized to the measured activity at day 0.

### Flow-Through Recirculating Bioreactor System

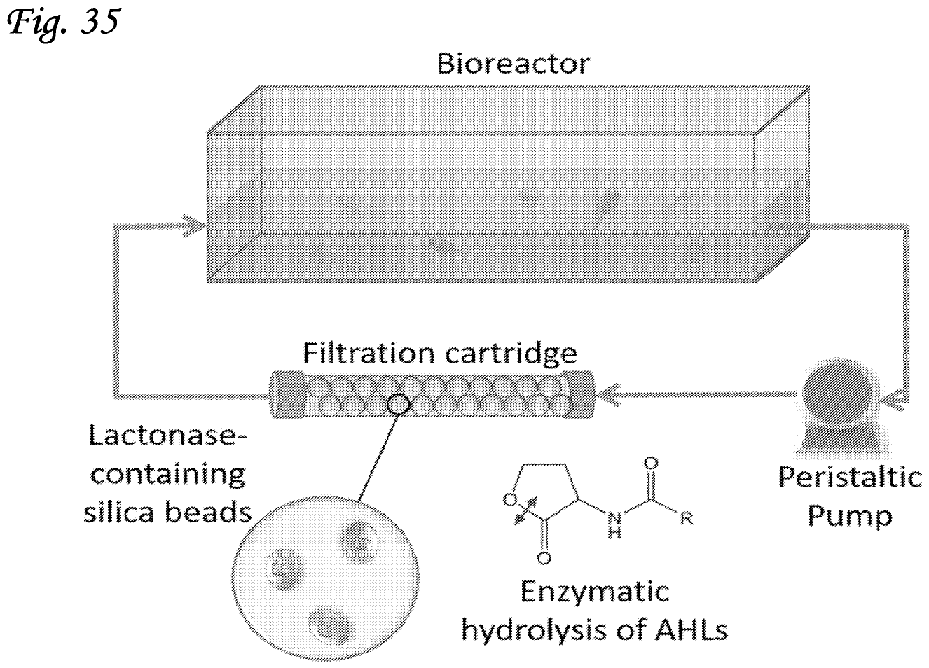

The flow-through system used in this study consisted of three 3-liter tanks set up in parallel. The parallel circuit was achieved through the use of a multi-channel peristaltic pump (Masterflex L/S, Cole-Parmer, USA) (FIG. 35). The flow rate was set to 18 mL/min and the peristaltic pump ensured even flow rate in each tank. The flow-through filtration cassette consists of a 10 mL chromatography column filled with QQ gel beads or control beads. Each tank contained three liters of 15× diluted LB media in water and a pre-broken 96-stripwell plate was submerged to the bottom (so that individual wells could be harvested for either biofilm quantification or DNA extraction). At the bottom of each bioreactor were eleven 22 mm square microscope cover slips to be later used for biofilm imaging. For the inoculum, about 5 grams of soil (Saint Paul, Minn., USA) was re-suspended in 40 mL of water. After a homogenous mixture was achieved, the suspension was lightly centrifuged (5 min, 500×g) and 200 μL of the cloudy supernatant was added to 30 mL of LB media and allowed to grow overnight (16 hours, 37° C., 200 RPM). 10 mL of this overnight soil culture was inoculated into each tank and the system was allowed to run for 21 days at room temperature. During this time, measurements were taken every day to monitor OD_{600 nm} of the water, pH of the water, and amount of biofilm present on multiple surfaces.

### Biofilm Quantification

Inside each of the tanks sat two submerged 96-stripwell plates. These plates were pre-broken so that individual wells could be extracted every day for measurements. Individual wells were extracted in triplicate for crystal violet biofilm quantification (OD_{550 nm}) using a similar protocol as previously described¹³. Wells were drained of excess fluid and loose cells, and then stained with 125 μL of 0.1% crystal violet solution for 15 minutes at room temperature. The crystal violet stain was then rinsed away with water and the wells were dried upside down overnight. To quantify biofilm, the remaining crystal violet stained to the wells was solubilized with 125 μL of 30% acetic acid and this solution was read on a spectrophotometer at 550 nm. 125 μL of 30% acetic acid was used as a blank. The optical density at 600 nm was also used to assess the planktonic growth in the tank were measured by reading a 200 μL sample with a 96 well plate spectrophotometer.

### pH Measurements

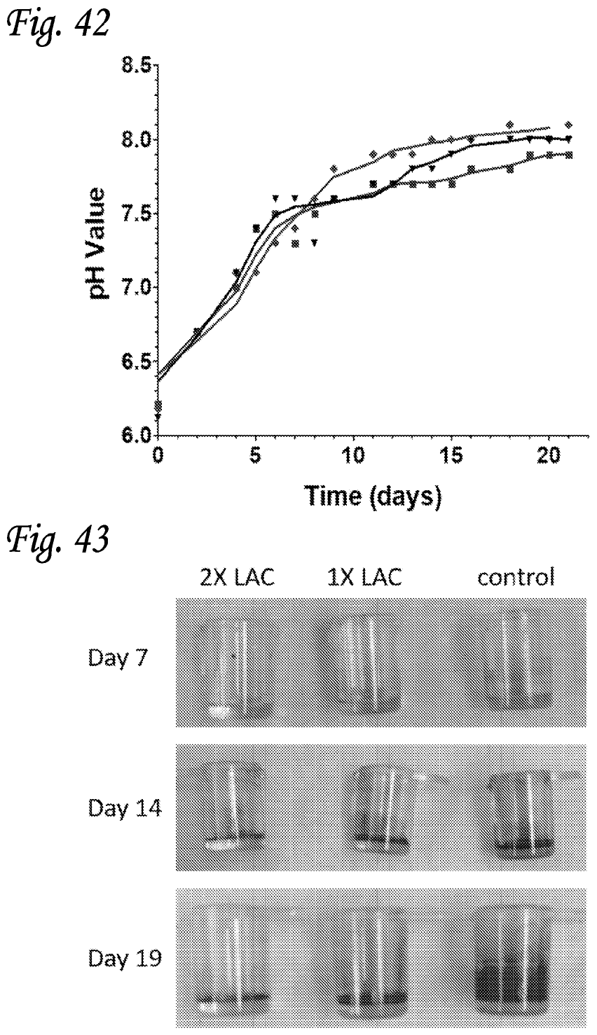

Tank pH values were measured with a portable probe (accuracy to ±0.05 pH units) that could be sterilized between the measuring of each tank. The pH of each tank was monitored throughout the experiment (FIG. 42).

### Sample Preparation for Imaging

Microscope cover glasses (Fisher Scientific) were submerged inside the tanks. These were harvested for biofilm visualization analysis on a Zeiss confocal microscope (West Germany). The cover slips were fixed with 2% paraformaldehyde in 1×PBS for one hour at room temperature. The slips were then rinsed twice with 1×PBS and end-fixed in a solution of 50% EtOH, 50% 1×PBS. These samples were then stored at −20° C. for later processing. To prepare the slips for imaging, the stored samples were washed twice with 1×PBS and stained with 1× Sybr Gold nucleic acid stain for ten minutes at room temperature. Slips were then washed with 100% EtOH and mounted onto microscope slides for fluorescence analysis. A 1:4 mixture of Citifluor:Vectashield was used for mounting media.

### Replication of the Effects of the Lactonase on a Complex Community

Inoculum were prepared by re-suspending about 5 grams of soil (taken outside GortnerLab building, Saint Paul Campus, Saint Paul, Minn., USA) in 40 mL of water. After a homogenous mixture was achieved, the suspension was lightly centrifuged (5 min, 500×g) and 200 uL of the cloudy supernatant was added to 30 mL of LB media and allowed to grow overnight. This inoculum was used to inoculate 15× diluted LB media (in water) cultures. Replicate cultures (5 mL each; in 50 mL tube) were incubated at 25° C. and treated by adding to the culture 2.5 mg of enzymes (0.5 mg/mL final), with the inactive mutant SsoPox 5A8 and with the improved mutant SsoPox W263I. Samples were collected for DNA extraction after 3 and 7 days.

### Microbial Community Analysis (MiSeq)

DNA extractions were carried out on biofilms. Submerged wells from the strip-well plates were drained of excess cells/water. Biofilm was scraped from the polypropylene well and put into Powerbead® tubes for DNA extraction (MoBio Powersoil® DNA Extraction Kit). Purified DNA samples were submitted to the University of Minnesota's Genomics Center for 16S rRNA sequencing. Using the Genomics Center platform, each sample underwent amplification, indexing, normalization, pooling, size selection, and final QC for sequencing. The V4 region of the 16s rRNA gene was amplified using primer 515f (5′-GTGCCAGCMGCCGCGGTAA-3′ (SEQ ID NO:14)). After the library preparation steps, it was confirmed all samples passed QC and were submitted for sequencing.

### Sequencing Data Analysis

All samples were processed using Mothur v1.35.1. For each sample, 1,250 sequences were used for final analyses. Genus-level identification was achieved for the composition of the bacterial community. Analysis of similarity (ANOSIM) and analysis of molecular variances (AMOVA) were used to evaluate the beta diversity (community composition) among samples using Bray-Curtis dissimilarity matrices (BC) (Bray & Curtis 1957; Clarke 1993). Ordination of Bray-Curtis matrices was performed using principal coordinate analysis (PCoA) to further analyze diversity of sample days throughout the tank (Anderson & Willis 2003). To visualize the distribution of taxonomies and diversities in microbial communities among the samples, R v3.3.1 was used to conduct the normalized relative abundance and OTUs at genus level⁴⁵. All of alpha values evaluated at α=0.05.

### Results and Discussion

### Engineered Lactonase-expressing Cells Entrapped in Silica Capsules

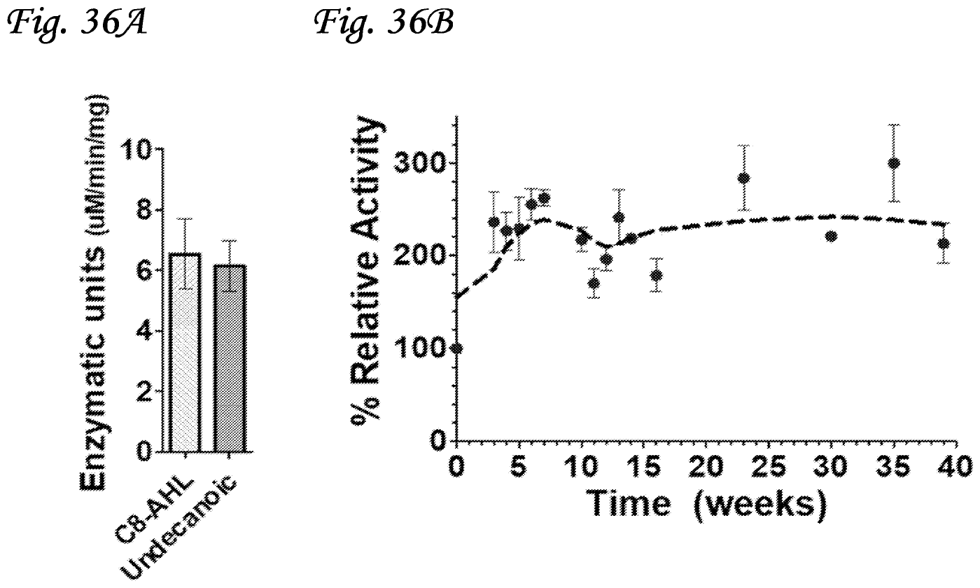

Silica encapsulation is a method of choice for entrapping biological materials such as enzymes or cells, due to their mechanical properties, durability, stability, cost, and synthesis in conditions compatible with biological molecules^{38, 46-48} Silica gels were previously used to encapsulate bioreactive bacteria for bioremediation^{38, 39, 42, 43}. While most encapsulated bacteria may remain viable through the process of making the gels⁴⁹, it is likely to be unnecessary in this study, since the lactonase SsoPox is a metalloenzyme that only requires a water molecule as the nucleophile for the hydrolytic reaction¹⁹. Therefore, cells can be seen as “bags of enzymes” that disrupt the signaling molecules produced by bacteria. We demonstrate that the obtained silica gels show catalytic activity against various lactones including C8-AHL and γ-undecanoic lactone, consistently with the enzyme activity in solution (FIG. 36A). The measured activity demonstrates that the lactonase overproduced in E. coli cells is active inside the beads, and that lactones with various acyl chain length can access its active site. The lactonase assay used in this study is a pH-based assay that we previously described^{29, 31, 32}. While this assay allows for the monitoring of the lactone ring opening (the latter generating a proton), it requires significant optimization of the activity buffer for each measurement due to the buffering capacity of the gel. In order to conveniently evaluate the durability of the silica gels over time, we used the chromogenic substrate paraoxon as a proxy for SsoPox activity. Indeed, SsoPox is a native lactonase with promiscuous ability to degrade phosphotriesters, and is capable of hydrolyzing paraoxon19, 22, 23. Using this assay, we demonstrated that the lactonase containing gel remains active for at least 39 weeks (˜9 months; FIG. 36B). This is a longer time than previous studies on atrazine degradation performed with a different enzyme but in similar conditions (4 months)³⁸. The observed durability is consistent with the extreme stability of SsoPox W263I, that remains stable for >300 days (˜10 months) at 25° C. as a purified protein sample¹⁶. Interestingly, the activity of the enzymatic gel at To increases over the course of the first 5 weeks of the experiment (˜3-fold). This may be caused by a change in the structure or porosity of the silica gel that could lead to an increased diffusion of the substrate into the enzyme active sites, and may suggest that our current gel formulation could be optimized in future studies. Our successful obtaining of silica gels containing engineered, overexpressed lactonases opens up a lot of new possibilities to study signal disruption in microbial communities. Control on expression level, ability to engineer the lactonase, or swap for a different lactonase isolated from another organism will be useful to optimize quorum quenching in complex contexts. Additionally, because this technology does not require purified enzyme, it may allow for the production of highly potent, specific, water filtration beads to inhibit biofilms and biofouling at low cost.

### Silica Beads Containing Lactonase Enzyme Inhibit Biofilm Formation of Complex Microbial Communities in a Water Recirculating System

We have created a water recirculating system where the bacterial community from a soil sample was cultured. The water was pumped through a filtration cartridge filled up with silica capsules (FIG. 35). We used two types of silica beads, (i) beads where E. coli cells overexpressing the lactonase SsoPox W263I are entrapped, dubbed lactonase beads, and (ii) beads where E. coli cells overexpressing a control protein (inactive mutant 5A8) are entrapped, dubbed control beads. Experimental design consisted of three independent bioreactors running in parallel: one setup used lactonase beads only (labelled 2× lactonase), the second setup used a filtration cartridge composed of a 1:1 ration of lactonase beads and control beads (labelled 1× lactonase), while the third setup filtration cartridge only contained control beads (labelled control). The water soluble AHLs produced by the microbial community growing in the tank are therefore being filtered through this cartridge, and degraded by the lactonase enzyme.

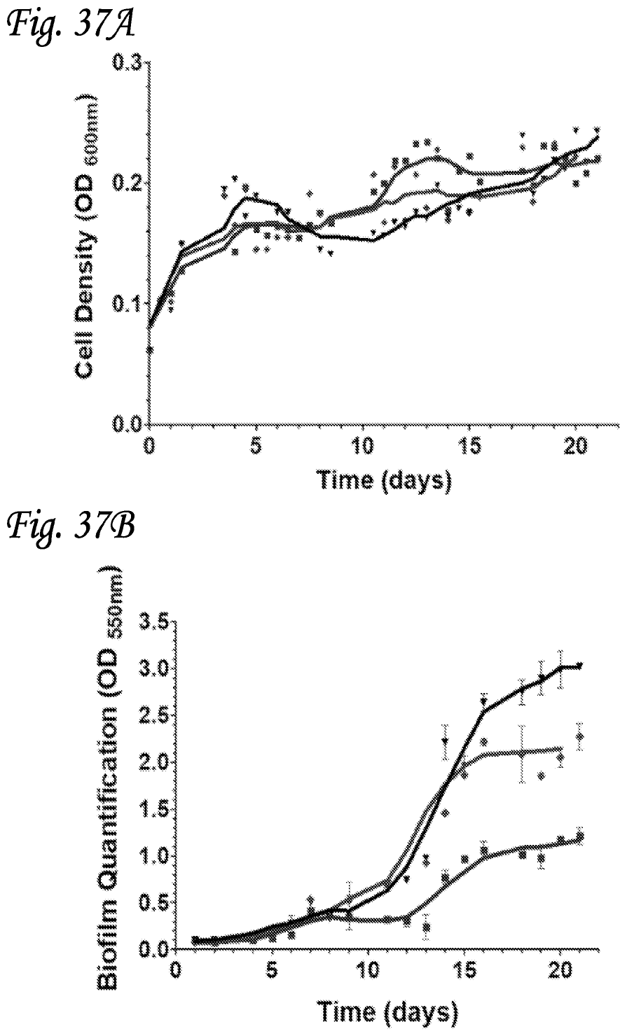

Effects of the action of the lactonase enzyme in the filtration cartridge were monitored at different levels: the pH of the media, as well as the optical density at 600 nm were recorded during the time course of the experiments. The pH of the media has been increased from a starting value of ˜6.2 to a final value of ˜8 in all three experimental setups (FIG. 42). Similarly, the OD600 nm, used as a proxy for cell density, has been mildly increasing over the course of the experiment in a similar fashion in the three bioreactors (FIG. 37A).

### Biofilm Formation Formed Over the Time Course of the Experiment in the Bioreactors.

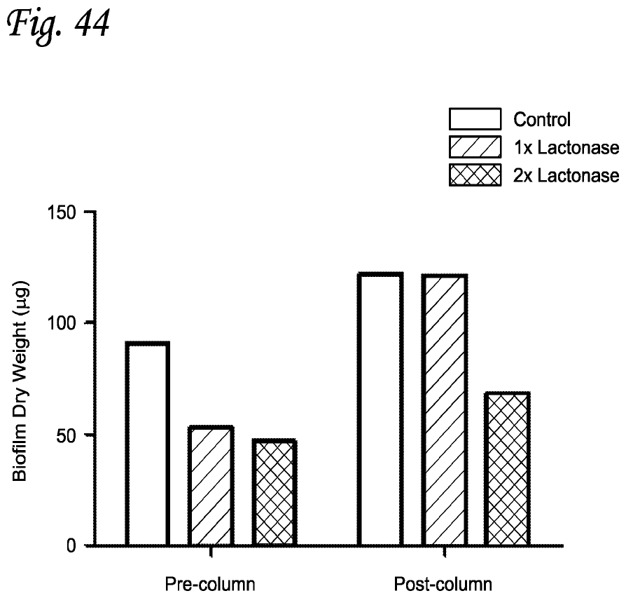

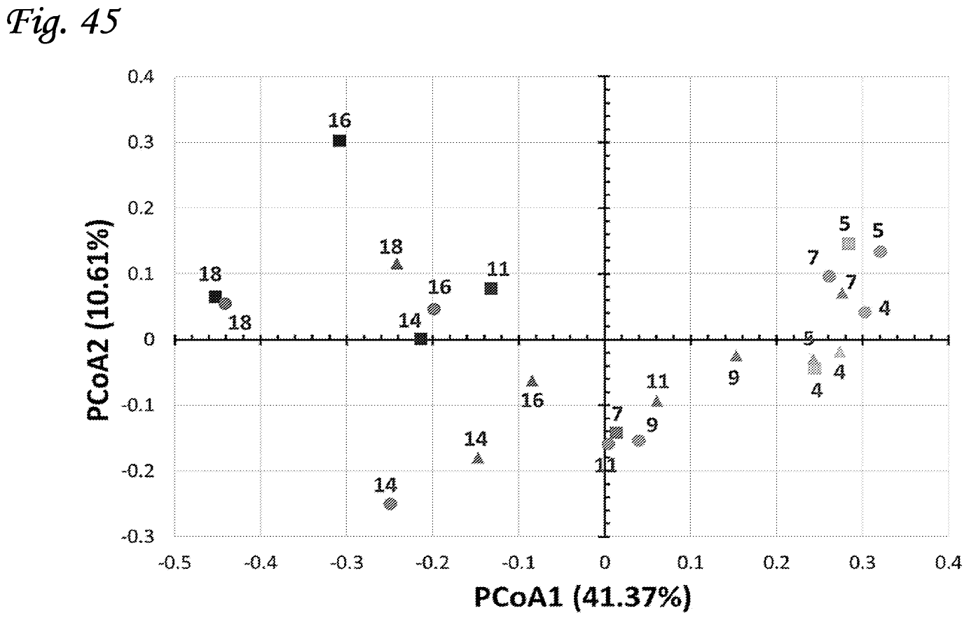

Biofilm was also quantified in the three bioreactors, over time (FIG. 37B). Submerged plastic wells were sampled and assayed using crystal violet dye (FIG. 43). Measurements indicated that biofilms were slowly forming during the first 11 days of the experiments, and then accelerated in all bioreactors. Interestingly, there are no significant difference in our biofilm quantification during the first 11 days of the experiment between filtration systems using lactonase or control beads. However, the measurements after 23 days indicate a reduction of at least 50% of the formed biofilm in presence of the lactonase beads, as compared to control. Reduction factor might be larger, since OD600 nm measurements reached saturation in the control experiment. However, this reduction factor is consistent with the observed reduction in biofilm dry weight in tubings (49 to 44% when comparing control and 2× lactonases after 21 days for pre-column and post column tubing, respectively (FIG. 44) Remarkably, Inhibition of biofilm is observed as a function of the lactonase concentration in the cartridge: inhibition is larger in the 2× than with the 1× concentration (FIG. 37C). This suggests that the lactonase activity may have been limiting, and/or that the design of the experiment was sub-optimal (e.g. flow rate).

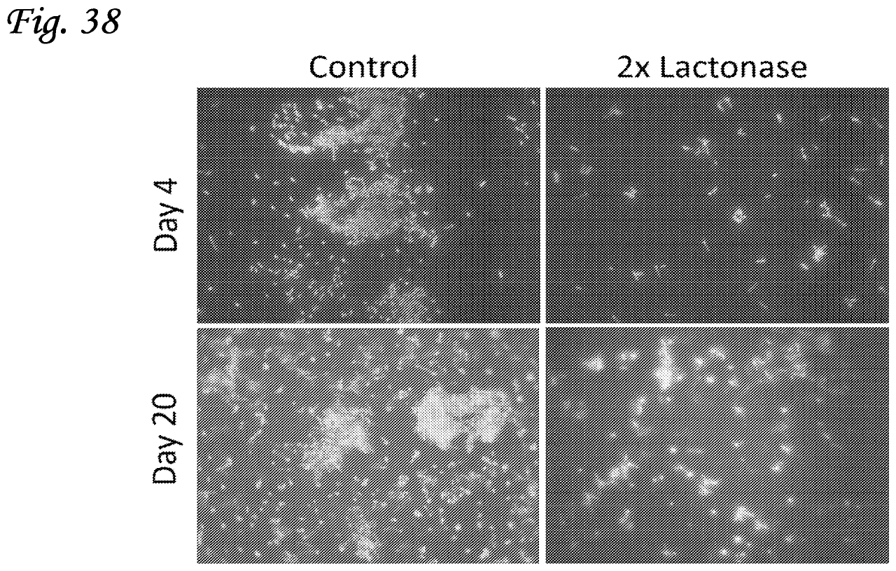

Biofilm forming in the bioreactors was also imaged in the early stage of the experiment (day 4) and in the late stage of the experiment (day 20) (FIG. 38). DNA staining of the submerged microscopy slips reveals that the presence of lactonase in the filtration cartridge lead to a reduction in the adhesion of cells on the surface of the slips. While we weren't able to stain or visualize the matrix in this experiment, it is apparent that the control tank biofilm has more structure and maturity than that of the lactonase treated tank. Interestingly, this reduction of cell attachment is also observed in the early stage of biofilm formation (day 4). Interestingly, the importance of signaling in the biofilm attachment step was previously described⁵¹, even if literature extensively described the importance of signaling in the biofilm maturation step⁵¹. Imaging results are consistent with the obtained biofilm quantification data.

Observations that lactonase beads can effectively reduce biofilm formation of a complex microbial community is consistent with previous observations using encapsulated microbes naturally expressing lactonases in MBR systems⁹. However, the demonstration in this study of the ability of entrapped lactonases to inhibit biofilm formation in a recirculating system opens new perspectives in water treatment. Additionally, it raises questions about the specific mechanism of action of entrapped lactonases on the microbial community signaling. Because lactonases are enzymes that degrade the secreted signaling molecules (AHLs), no physical contact between the enzyme molecules and bacteria is needed for its action. Yet, the question of the diffusability of AHLs in various media is interesting and will need to be investigated as it may modulate the “action range” of the various AHLs, and consequently, of lactonases.

### Presence of a Lactonase Induces Changes in the Biofilm Microbial Composition

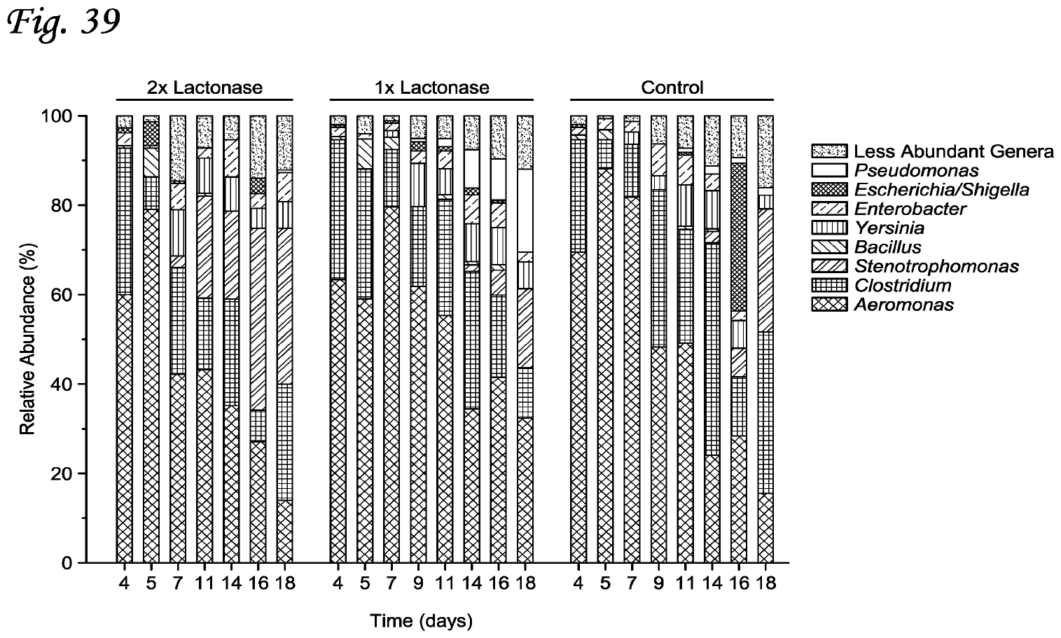

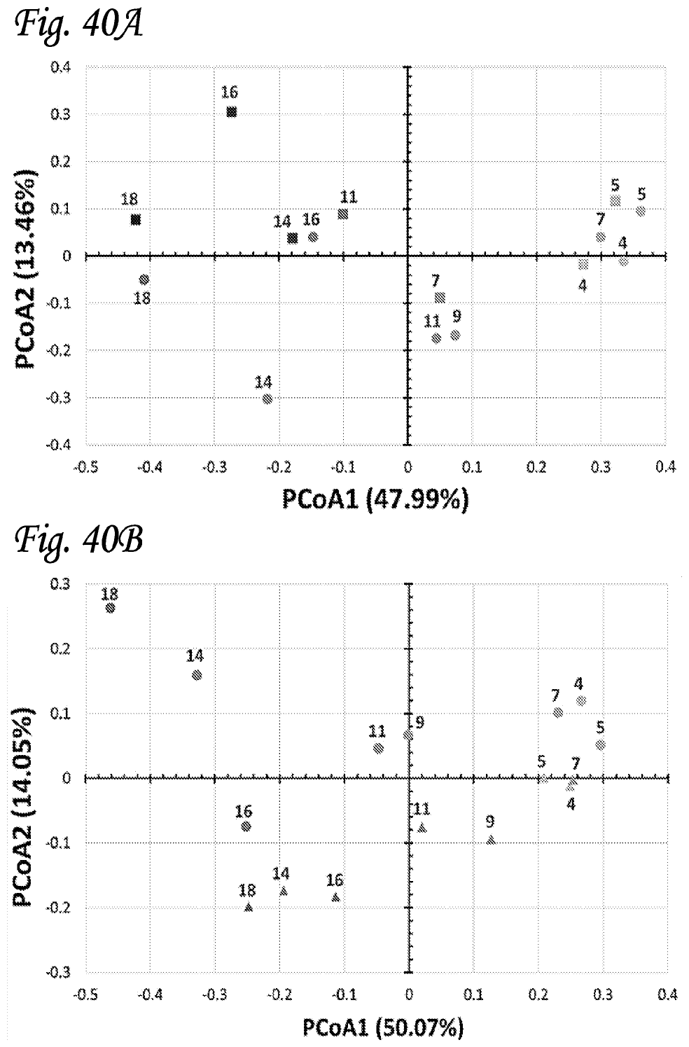

Biofilm samples from the three different bioreactors were submitted to sequencing and community composition determination. Samples were collected over the time course of the experiment to evaluate the population dynamics in the different setups. Given the low diversity of the samples (less abundant group <10%), we considered 1250 sequences for each sample. Analysis of sequencing data to the genus-level (FIG. 39) and Principle Coordinate Analysis (FIGS. 38 and 43) reveal that communities in all three setups are very similar in the early stages of the experiment. This is to be expected because all three bioreactors were inoculated with the same starting culture. However, notable differences are visible from day 7. In the bioreactor treated with the highest concentration of lactonase (2×), Aeromonas (Gram negative) represented 42.16% of the community on day 7, while it is 79.68% and 81.68% of the communities in the lactonase (1×) and in the control treatments, respectively (FIG. 39A). Principle Coordinate Analysis also highlights that community compositions start to separate from control from day 7 (FIG. 39B).

Other notable differences include the relative populations of Stenotrophomonas (Gram negative), Pseudomonas (Gram negative), and Clostridium XIVa (Gram positive). For instance, the 2× lactonase bioreactor shows the introduction (day 7) and sustained presence of Stenotrophomonas much earlier than that of the 1× Lactonase and control bioreactors (on day 14). In the 1× Lactonase bioreactor, we observe a rise of the Pseudomonas population at day 11 and a gradual increase in its abundance within the community throughout the rest of the experiment. Lastly, the control bioreactor hosted a larger Clostridium population than the two other bioreactors during the second part of the experiment (days 9 to 18).

Presence of a Lactonase Modulates Diversity within Genera but not the Community Diversity

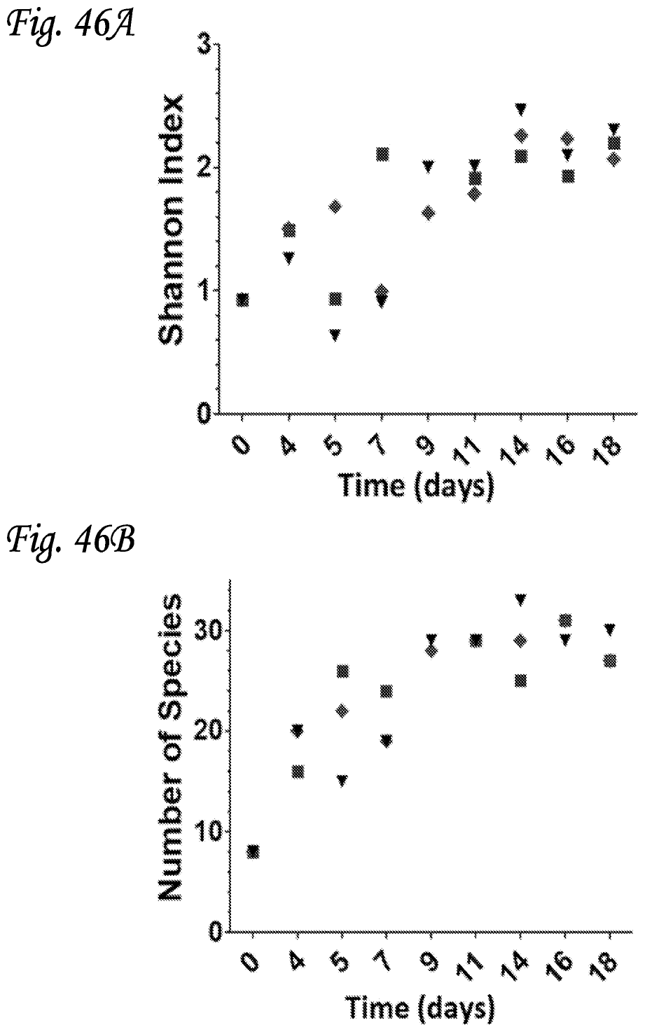

The analysis of both the relative abundance and the diversity of each genus distinctly highlights the population changes as a function of lactonase concentration and time. Overall, this analysis shows that the presence of the lactonase induces changes in the relative abundance and diversity of genera, but does not seem to significantly alter the overall community diversity. This is further evidence by Shannon indexes values and observed species count (FIG. 46). Additionally, this representation confirms the previous observation made on Stenotrophomonas, Pseudomonas and Clostridium XVa that are specifically enriched over time in the 2× lactonase, the 1× lactonase, and the control bioreactors, respectively, as compared to the two other setups. Data shows that this increasing proportion of these genera in the community is concomitant with an increase in their diversity.

This detailed analysis of the communities' compositions reveals some low abundance genera that are specific to treatments. For example, Propionispora are only detected in setups using lactonase in the filtration cartridge, whereas Acetivibrio are only detected in the control bioreactor. Other microbial community biases are visible: Achromobacter are more abundant in the setups using lactonases, as compared to control, whereas Sporomusa's abundance and diversity is decreasing when the concentration of lactonase is increasing.

These observed changes induced by the action of lactonases are consistent with a previous study performed in the context of membrane biofouling³⁷, as well as reports indicating that lactonase can change composition of gut microbiomes in fish⁵². Mechanisms underlining the ability of quorum quenching lactonases to affect complex communities are unknown. Complete QS circuits (a synthase, and a receptor) were previously reported to be found only in proteobacteria⁵³. Within bacteria genera detected in this study, some are known to produce AHLs and utilize them for sensing (i.e. (Pseudomonas, Aeromonas, Yersinia, ⁵⁴⁻⁵⁷), some are known to be capable of producing AHLs (i.e. Enterobacter^{58, 59}), some are known to be capable of sensing AHLs (i.e. Stenotrophomonas, Escherichia, Shigella⁶⁰⁻⁶³), and some are not known to produce, use or sense AHLs (i.e. Clostridium) (Table 11). Additionally, relationships between the presence of the lactonase and some genus known to be affected by it (e.g. Pseudomonas⁵⁷) may not be straightforward, as indicated by the increase of Pseudomonas in the community of the 1× lactonase setup. Furthermore, it is intriguing to note that Clostridium XIVa, despite being a gram positive bacteria that is not known to produce and/or sense AHLs, is reduced in presence of the lactonase. This observation fits previous observation describing the ability of lactonase to inhibit the biofilm of Staphylococcus aureus and Escherichia coli^{33,35}. Mechanisms explaining these observations are lacking and more studies will be necessary to derive the rules underlining these complex interactions.

TABLE 11
Production and sensing of AHLs in representative strains from the main genus
identified in the biofilm community.

| | Gram | | AHLs | AHLs | |
|---|---|---|---|---|---|
| Detected Genera | coloration | Example strain | production | receptor | ref |
| Acetivibrio | negative | N/A | N/A | N/A | N/A |
| Achromobacter | negative | Achromobacter | N/A | Yes, and | ^{1,2} |
| | | piechaudii | | encodes a | |
| | | | | lactonase | |
| Aeromonas | negative | Aeromonas | C4 AHL; | yes | ³ |
| | | hydrophilae | C6AHL | | |
| Clostridium | Positive | N/A | N/A | N/A | N/A |
| Stenotrophomonas | negative | Stenotrophomonas | N/A | yes | ⁴ |
| | | maltophilia | | | |
| Bacillus | positive | Bacillus subtilis | N/A | No receptor, | ⁵ |
| | | BS-1 | | but encodes | |
| | | | | lactonases | |
| Yersinia | negative | Yersinia | 25 different | yes | ^{6,7} |
| | | pseudotuberculosis | AHLs, most | | |
| | | | abundant | | |
| | | | are 3-oxo | | |
| | | | C6, C7 and | | |
| | | | C8 AHLs | | |
| Enterobacter | negative | Enterobacter sp. ; | C12 AHL | N/A | ^{8,9} |
| | | Enterobacter | | | |
| | | ludwigii | | | |
| | | EN-119 | | | |
| Escherichia/ | negative | Escherichia coli | No | yes | ¹⁰⁻¹² |
| Shigella | | | production | | |
| Propionispora | negative | N/A | N/A | N/A | N/A |
| Pseudomonas | negative | Pseudomonas | C4 AHL; | yes | ¹³ |
| | | aeruginosa | 3-oxo C12 | | |
| | | | AHL | | |
| Sporomusa | negative | N/A | N/A | N/A | N/A |

N/A (not applicable): data not available for this genera;
¹Kretzschmar et la., AIMS Env. Sci 2, 122-133 (2015);
²Swearingen et al., J. Bacteriol. 195, 173-179 (2013);
³Khajanchi et al., Infect. Immun. 79, 2646-2657 (2011);
⁴Martínez et al., Front. Cell. Infect. Microbiol. 5, 41 (2015);
⁵Pan et al., Microbiol. Res. 163, 711-716 (2008);
⁶Ortori et al., Anal. Bioanal. Chem. 387, 497-511 (2007);
⁷Medina-Martínez et al., J. Appl. Microbiol. 102, 1150-1158 (2007);
⁸Ochiai et al., Biosci. Biotechnol. Biochem. 77, 2436-2440 (2013);
⁹Yin et al., Sensors 12, 14307-14314 (2012);
¹⁰Lu et al., Front. Cell. Infect. Microbiol. 7, 7 (2017);
¹¹Taghadosi et al., Rep. Biochem. Mol. Biol. 3, 56 (2015);
¹²Soares et al., Curr. Opin. Microbiol. 14, 188-193 (2011);
¹³Venturi et al., FEMS Microbiol. Rev. 30, 274-291 (2005).

### Presence of a Lactonase Alters a Suspension Microbial Community

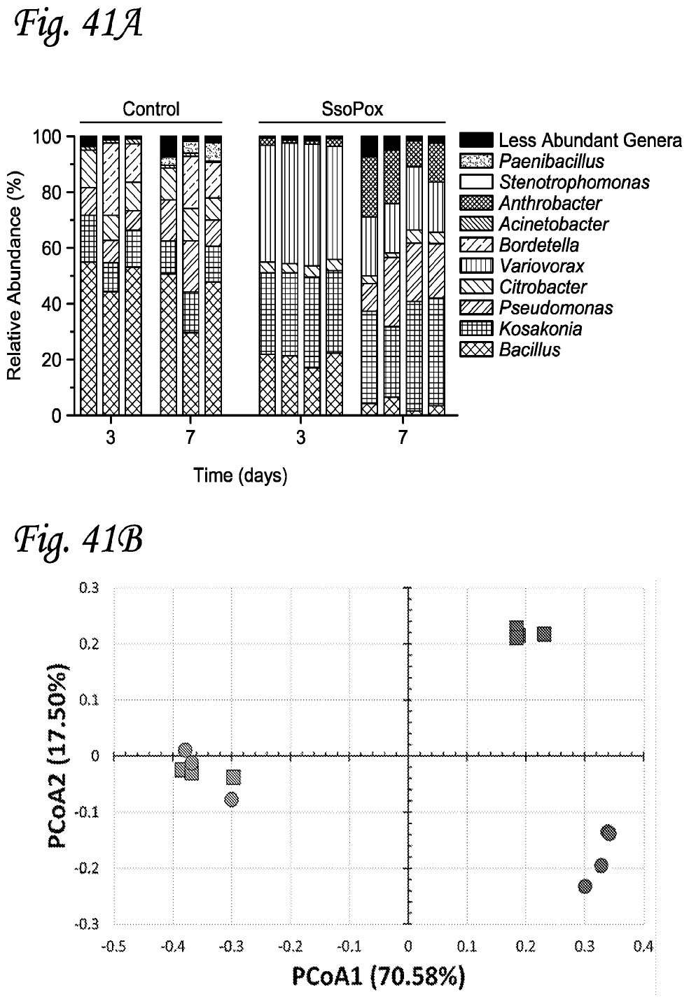

Our bioreactor shows that the presence of the lactonase can significantly alter the composition of a complex soil biofilm community. We decided to investigate the ability of a lactonase to change the composition of suspension community. Therefore, we cultured a complex soil community for up to 7 days and added both an active lactonase variant (Ssopox-W263I) and an inactive variant (Ssopox 5A8) as a control as quadruplicates. Samples (suspension culture) were collected after 3 and 7 days of culture. Given the low diversity of the samples (less abundant group <10%), we considered 1250 sequences for each sample. Analysis of sequencing data to the genus-level (FIG. 41A), Principle Coordinate Analysis (FIG. 41B) and statistics (Tables 12 and 13) reveal that communities are significantly different. While the observed changes induced by signal disruption on the biofilm community is consistent with the ability of lactonases to inhibit the formation of biofilms, it is unexpected to observe that these enzymes also alters the composition of bacteria growing in suspension. The importance of quorum sensing for bacterial fitness has been documented in numerous contexts^{64,65}, including biofilms⁶⁶. The disruption of QS by lactonases may modulate differentially bacterial fitness and account for the induced changes in the microbial population.

TABLE 12
AMOVA Statistical tests of suspension community sequencing data.

| Samples | statistical | | | | |
|---|---|---|---|---|---|
| comparison | factor | Among | Within | Total | p-value |
| Control_3- | SS | 1.655 | 0.144 | 1.798 | <0.001 |
| Control_7- | df | 3 | 10 | 13 | |
| SsoPox_3- | MS | 0.552 | 0.014 | | |
| SsoPox_7 | Fs | 38.393 | | | |
| Control_3- | SS | 0.032 | 0.093 | 0.125 | 0.279 |
| Control_7 | df | 1 | 4 | 5 | |
| | MS | 0.032 | 0.023 | | |
| | Fs | 1.378 | | | |
| Control_3- | SS | 0.624 | 0.041 | 0.665 | 0.035 |
| SsoPox_3 | df | 1 | 5 | 6 | |
| | MS | 0.624 | 0.008 | | |
| | Fs | 75.986 | | | |
| Control_3- | SS | 0.826 | 0.084 | 0.91 | 0.025 |
| SsoPox_7 | df | 1 | 5 | 6 | |
| | MS | 0.826 | 0.017 | | |
| | Fs | 49.02 | | | |
| Control_7- | SS | 0.618 | 0.059 | 0.677 | 0.033 |
| SsoPox_3 | df | 1 | 5 | 6 | |
| | MS | 0.618 | 0.012 | | |
| | Fs | 51.991 | | | |
| Control_7- | SS | 0.828 | 0.103 | 0.931 | 0.028 |
| SsoPox_7 | df | 1 | 5 | 6 | |
| | MS | 0.828 | 0.021 | | |
| | Fs | 40.36 | | | |
| SsoPox_3- | SS | 0.338 | 0.051 | 0.389 | 0.027 |
| SsoPox_7 | df | 1 | 6 | 7 | |
| | MS | 0.338 | 0.008 | | |
| | Fs | 39.779 | | | |

SS = sum of square; df = degrees of freedom; MS = mean square; Fs = F Statistics

TABLE 13
ANOSIM Statistical tests of suspension community sequencing data.

| Samples comparison | R-value | P-value |
|---|---|---|
| Control_3-Control_7-SsoPox_3-SsoPox_7 | 0.94 | <0.001* |
| Control_3-Control_7 | 0.11 | 0.387 |
| Control_3-SsoPox_3 | 1.00 | 0.026 |
| Control_3-SsoPox_7 | 1.00 | 0.028 |
| Control_7-SsoPox_3 | 1.00 | 0.025 |
| Control_7-SsoPox_7 | 1.00 | 0.029 |
| SsoPox_3-SsoPox_7 | 1.00 | 0.024 |

### Conclusions

This study aimed to create silica-based capsules with quorum quenching abilities and a potential for engineering. We used E. coli cells overexpressing an engineered, extremely stable and active lactonase, and these cells were entrapped in silica gels. The use of cells allows for potential controls on expression levels, the control on the type of lactonase used in the system, as well as future engineering in improving the lactonase properties. The use of silica gels provides physical protection of the enzyme from the environment, mechanical properties that are compatible with the use of these capsules as water filtration materials, and allows for the production at low costs. Our study demonstrates that lactonase-containing beads are reducing the biofilm formation of a complex soil microbial community, in a dose-dependent manner, in a water recirculating system. Biofilm inhibition is observed despite the abundant presence of microbes that are not known for using or sensing AHLs, such as Clostridium. Sequencing analysis revealed that the biofilm inhibition is concomitant to a change in the microbial community composition on the surface. Dynamic population analysis shows that the bias introduced by AHL signal disruption occurs rapidly and is persistent over the time course of the experiment. Changes induced in the biofilm population by AHL signal disruption do not only relate to changes in the relative proportion of some genera (e.g. Aeromonas, Clostridium, Stenotrophomonas) but also to the specific presence (e.g.) or absence (e.g.) of genus in the biofilm. Additionally, we find that the changes induced to the microbial community are (i) reproducible (ii) statistically significant and (iii) also relate to bacterial in the suspension. This unexpected finding is possible evidence for the importance of signaling in the competition between bacteria within communities. The designed system reported in this study provides a unique platform to study the importance of bacterial signaling, and the effects of signal disruption in complex communities. We are convinced that these findings and tools will pave the way for future investigations unravelling the potential of quorum quenching enzymes in the fields of water treatment.

### CITATIONS FOR EXAMPLE 7

1. Bassler, B. L. How bacteria talk to each other: regulation of gene expression by quorum sensing. Curr. Opin. Microbiol. 2, 582-587 (1999).
2. Miller, M. B. & Bassler, B. L. Quorum sensing in bacteria. Annu. Rev. Microbiol. 55, 165-199 (2001).
3. LaSarre, B. & Federle, M. J. Exploiting quorum sensing to confuse bacterial pathogens. Microbiol. Mol. Biol. Rev. 77, 73-111 (2013).
4. Costerton, J. W., Stewart, P. S. & Greenberg, E. P. Bacterial biofilms: a common cause of persistent infections. Science 284, 1318-1322 (1999).
5. Stewart, P. S. & Costerton, J. W. Antibiotic resistance of bacteria in biofilms. The lancet 358, 135-138 (2001).
6. Zhang, L. H. Quorum quenching and proactive host defense. Trends Plant Sci 8, 238-44 (2003).
7. Bzdrenga, J. et al. Biotechnological applications of quorum quenching enzymes. Chem. Biol. Interact. (2016).
8. Oh, H.-S. et al. Control of membrane biofouling in MBR for wastewater treatment by quorum quenching bacteria encapsulated in microporous membrane. Environ. Sci. Technol. 46, 4877-4884 (2012).
9. Kim, S.-R. et al. Biofouling control with bead-entrapped quorum quenching bacteria in membrane bioreactors: physical and biological effects. Environ. Sci. Technol. 47, 836-842 (2013).
10. Chow, J. Y., Yang, Y., Tay, S. B., Chua, K. L. & Yew, W. S. Disruption of biofilm formation by the human pathogen Acinetobacter baumannii using engineered quorum-quenching lactonases. Antimicrob. Agents Chemother. 58, 1802-1805 (2014).
11. Zhang, Y., Brackman, G. & Coenye, T. Pitfalls associated with evaluating enzymatic quorum quenching activity: the case of MomL and its effect on Pseudomonas aeruginosa and Acinetobacter baumannii biofilms. PeerJ5, e3251 (2017).
12. Dong, Y. H., Xu, J. L., Li, X. Z. & Zhang, L. H. AiiA, an enzyme that inactivates the acylhomoserine lactone quorum-sensing signal and attenuates the virulence of Erwinia carotovora. Proc Natl Acad Sci UA 97, 3526-31 (2000).
13. Hraiech, S. et al. Inhaled Lactonase Reduces Pseudomonas aeruginosa Quorum Sensing and Mortality in Rat Pneumonia. PLoS One 9, e107125 (2014).
14. Guendouze, A. et al. Effect of Quorum Quenching Lactonase in Clinical Isolates of Pseudomonas aeruginosa and Comparison with Quorum Sensing Inhibitors. Front. Microbiol. 8, (2017).
15. Lee, S. et al. Crossing the border between laboratory and field: Bacterial quorum quenching for anti-biofouling strategy in an MBR. Environ. Sci. Technol. 50, 1788-1795 (2016).
16. Remy, B. et al. Harnessing hyperthermostable lactonase from Sulfolobus solfataricus for biotechnological applications. Sci. Rep. 6, (2016).
17. Merone, L., Mandrich, L., Rossi, M. & Manco, G. A thermostable phosphotriesterase from the archaeon Sulfolobus solfataricus: cloning, overexpression and properties. Extrem. Life Extreme Cond. 9, 297-305 (2005).
18. Elias, M. et al. Crystallization and preliminary X-ray diffraction analysis of the hyperthermophilic Sulfolobus solfataricus phosphotriesterase. Acta Crystallogr Sect F Struct Biol Cryst Commun 63, 553-5 (2007).
19. Elias, M. et al. Structural basis for natural lactonase and promiscuous phosphotriesterase activities. J Mol Biol 379, 1017-28 (2008).
20. Afriat, L., Roodveldt, C., Manco, G. & Tawfik, D. S. The latent promiscuity of newly identified microbial lactonases is linked to a recently diverged phosphotriesterase. Biochemistry (Mosc.) 45, 13677-86 (2006).
21. Elias, M. & Tawfik, D. S. Divergence and Convergence in Enzyme Evolution: Parallel Evolution of Paraoxonases from Quorum-quenching Lactonases. J Biol Chem 287, 11-20 (2012).
22. Hiblot, J., Gotthard, G., Elias, M. & Chabriere, E. Differential active site loop conformations mediate promiscuous activities in the lactonase SsoPox. PLoS One 8, e75272 (2013).
23. Hiblot, J., Gotthard, G., Chabriere, E. & Elias, M. Characterisation of the organophosphate hydrolase catalytic activity of SsoPox. Sci Rep 2, (2012).
24. Del Vecchio, P. et al. Structural determinants of the high thermal stability of SsoPox from the hyperthermophilic archaeon Sulfolobus solfataricus. Extremophiles 13, 461-70 (2009).
25. Jacquet, P. et al. Rational engineering of a native hyperthermostable lactonase into a broad spectrum phosphotriesterase. Sci. Rep. 7, 16745 (2017).
26. Tang, K. et al. MomL, a novel marine-derived N-acyl homoserine lactonase from Muricauda olearia. Appl. Environ. Microbiol. 81, 774-782 (2015).
27. Mascarenhas, R. et al. Structural and biochemical characterization of AidC, a quorum-quenching lactonase with atypical selectivity. Biochemistry (Mosc.) 54, 4342-4353 (2015).
28. Hiblot, J., Bzdrenga, J., Champion, C., Chabriere, E. & Elias, M. Crystal structure of VmoLac, a tentative quorum quenching lactonase from the extremophilic crenarchaeon Vulcanisaeta moutnovskia. Sci Rep 5, 8372 (2015).
29. Hiblot, J., Gotthard, G., Chabriere, E. & Elias, M. Structural and Enzymatic characterization of the lactonase SisLac from Sulfolobus islandicus. PLoS One 7, e47028 (2012).
30. Bzdrenga, J. et al. SacPox from the thermoacidophilic crenarchaeon Sulfolobus acidocaldarius is a proficient lactonase. BMC Res Notes 7, 333 (2014).
31. Bergonzi, C., Schwab, M., Chabriere, E. & Elias, M. The quorum-quenching lactonase from Alicyclobacter acidoterrestris: purification, kinetic characterization, crystallization and crystallographic analysis. Acta Crystallogr. Sect. F Struct. Biol. Commun. 73, (2017).
32. Bergonzi, C., Schwab, M. & Elias, M. The quorum-quenching lactonase from Geobacillus caldoxylosilyticus: purification, characterization, crystallization and crystallographic analysis. Acta Crystallogr F Struct Biol Commun 72, 681-6 (2016).
33. Ivanova, K. et al. Quorum-quenching and matrix-degrading enzymes in multilayer coatings synergistically prevent bacterial biofilm formation on urinary catheters. ACS Appl. Mater. Interfaces 7, 27066-27077 (2015).
34. Ivanova, K., Fernandes, M. M., Mendoza, E. & Tzanov, T. Enzyme multilayer coatings inhibit Pseudomonas aeruginosa biofilm formation on urinary catheters. Appl. Microbiol. Biotechnol. 99, 4373-4385 (2015).
35. Mayer, C., Romero, M., Muras, A. & Otero, A. Aii20J, a wide-spectrum thermostable N-acylhomoserine lactonase from the marine bacterium Tenacibaculum sp. 20J, can quench AHL-mediated acid resistance in Escherichia coli. Appl. Microbiol. Biotechnol. 99, 9523-9539 (2015).
36. Kim, S.-R. et al. Macroencapsulation of quorum quenching bacteria by polymeric membrane layer and its application to MBR for biofouling control. J. Membr. Sci. 473, 109-117 (2015).
37. Jo, S. J. et al. Effects of Quorum Quenching on the Microbial Community of Biofilm in an Anoxic/Oxic M BR for Wastewater Treatment. J. Microbiol. Biotechnol. 26, 1593-1604 (2016).
38. Reitegui, E. et al. Silica gel-encapsulated AtzA biocatalyst for atrazine biodegradation. Appl. Microbiol. Biotechnol. 96, 231-240 (2012).
39. Aukema, K. G., Kasinkas, L., Aksan, A. & Wackett, L. P. Use of Silica-Encapsulated Pseudomonas sp. Strain NCIB 9816-4 in Biodegradation of Novel Hydrocarbon Ring Structures Found in Hydraulic Fracturing Waters. Appl. Environ. Microbiol. 80, 4968-4976 (2014).
40. Mutlu, B. R., Yeom, S., Wackett, L. P. & Aksan, A. Modelling and optimization of a bioremediation system utilizing silica gel encapsulated whole-cell biocatalyst. Chem. Eng. J. 259, 574-580 (2015).
41. Mutlu, B. R., Yeom, S., Tong, H.-W., Wackett, L. P. & Aksan, A. Silicon alkoxide cross-linked silica nanoparticle gels for encapsulation of bacterial biocatalysts. J. Mater. Chem. A 1, 11051-11060 (2013).
42. Sakkos, J. K., Mutlu, B. R., Wackett, L. P. & Aksan, A. Adsorption and biodegradation of aromatic chemicals by bacteria encapsulated in a hydrophobic silica gel. ACS Appl. Mater. Interfaces 9, 26848-26858 (2017).
43. Sakkos, J. K., Kieffer, D. P., Mutlu, B. R., Wackett, L. P. & Aksan, A. Engineering of a silica encapsulation platform for hydrocarbon degradation using Pseudomonas sp. NCIB 9816-4. Biotechnol. Bioeng. 113, 513-521 (2016).
44. Gotthard, G., Hiblot, J., Gonzalez, D., Elias, M. & Chabriere, E. Structural and enzymatic characterization of the phosphotriesterase OPHC2 from Pseudomonas pseudoalcaligenes. PLoS One 8, e77995 (2013).
45. Al-Masaudi, S. et al. A metagenomics investigation of carbohydrate-active enzymes along the gastrointestinal tract of Saudi sheep. Front. Microbiol. 8, 666 (2017).
46. Nassif, N. et al. A sol-gel matrix to preserve the viability of encapsulated bacteria. J. Mater. Chem. 13, 203-208 (2003).
47. Idrees, M., Eqani, S. A. M. A. S. & Bokhari, H. Sol-gel immobilisation of methyl parathion degrading bacteria isolated from agricultural areas of Pakistan. Chem. Ecol. 29, 733-744 (2013).
48. Livage, J., Coradin, T. & Roux, C. Encapsulation of biomolecules in silica gels. J. Phys. Condens. Matter 13, R673 (2001).
49. Benson, J. J., Sakkos, J. K., Radian, A., Wackett, L. P. & Aksan, A. Enhanced biodegradation of atrazine by bacteria encapsulated in organically modified silica gels. J. Colloid Interface Sci. 510, 57-68 (2018).
50. Tomlin, K. L. et al. Quorum-sensing mutations affect attachment and stability of Burkholderia cenocepacia biofilms. Appl. Environ. Microbiol. 71, 5208-5218 (2005).
51. Parsek, M. R. & Greenberg, E. P. Sociomicrobiology: the connections between quorum sensing and biofilms. Trends Microbiol 13, 27-33 (2005).
52. Zhou, S., Zhang, A., Yin, H. & Chu, W. Bacillus sp. QSI-1 modulate quorum sensing signals reduce Aeromonas hydrophila level and alter gut microbial community structure in fish. Front. Cell. Infect. Microbiol. 6, (2016).
53. Case, R. J., Labbate, M. & Kjelleberg, S. AHL-driven quorum-sensing circuits: their frequency and function among the Proteobacteria. ISME J. 2, 345 (2008).
54. Khajanchi, B. K., Kirtley, M. L., Brackman, S. M. & Chopra, A. K. Immunomodulatory and protective roles of quorum-sensing signaling molecules N-acyl homoserine lactones during infection of mice with Aeromonas hydrophila. Infect. Immun. 79, 2646-2657 (2011).
55. Ortori, C. A. et al. Comprehensive profiling of N-acylhomoserine lactones produced by Yersinia pseudotuberculosis using liquid chromatography coupled to hybrid quadrupole-linear ion trap mass spectrometry. Anal. Bioanal. Chem. 387, 497-511 (2007).
56. Medina-Martinez, M., Uyttendaele, M., Meireman, S. & Debevere, J. Relevance of N-acyl-L-homoserine lactone production by Yersinia enterocolitica in fresh foods. J. Appl. Microbiol. 102, 1150-1158 (2007).
57. Venturi, V. Regulation of quorum sensing in Pseudomonas. FEMS Microbiol. Rev. 30, 274-291 (2005).
58. Ochiai, S. et al. Production and degradation of N-acylhomoserine lactone quorum sensing signal molecules in bacteria isolated from activated sludge. Biosci. Biotechnol. Biochem. 77, 2436-2440 (2013).
59. Yin, W.-F. et al. N-acyl homoserine lactone production by Klebsiella pneumoniae isolated from human tongue surface. Sensors 12, 3472-3483 (2012).
60. Martinez, P. et al. Stenotrophomonas maltophilia responds to exogenous AHL signals through the LuxR solo SmoR (Smlt1839). Front. Cell. Infect. Microbiol. 5, 41 (2015).
61. Lu, Y. et al. Quorum sensing N-acyl homoserine lactones-SdiA suppresses Escherichia coli-Pseudomonas aeruginosa conjugation through inhibiting traI expression. Front. Cell. Infect. Microbiol. 7, 7 (2017).
62. Taghadosi, R., Shakibaie, M. R. & Masoumi, S. Biochemical detection of N-Acyl homoserine lactone from biofilm-forming uropathogenic Escherichia coli isolated from urinary tract infection samples. Rep. Biochem. Mol. Biol. 3, 56 (2015).
63. Soares, J. A. & Ahmer, B. M. Detection of acyl-homoserine lactones by Escherichia and Salmonella. Curr. Opin. Microbiol. 14, 188-193 (2011).
64. Heurlier, K., Denervaud, V. & Haas, D. Impact of quorum sensing on fitness of Pseudomonas aeruginosa. Int. J. Med. Microbiol. 296, 93-102 (2006).
65. Darch, S. E., West, S. A., Winzer, K. & Diggle, S. P. Density-dependent fitness benefits in quorum-sensing bacterial populations. Proc. Natl. Acad. Sci. 109, 8259-8263 (2012).
66. Nadell, C. D., Xavier, J. B., Levin, S. A. & Foster, K. R. The evolution of quorum sensing in bacterial biofilms. PLoS Biol. 6, e14 (2008).

### Example 8

### Different Lactonases can Change Differentially the Composition of Microbiomes

The same experiments as described above were performed on the same soil microbial community but with two different lactonases, GcL, and Ssopox W263I. These enzymes differ primarily by their substrate specificity. Indeed, while GcL is a generalist that proficiently degrades lactones ranging from C4 to C12 AHLs, Ssopox W263I prefers longer acyl chain lactones, and is not capable of degrading short chain AHLs. This is important because different bacteria use different AHLs to communicate. Indeed, The structure of AHLs vary a lot with respect to the length of N-acyl chains (from C4 to C18), the hydroxyl or oxo group of the acyl chain and the saturated or unsaturated state of the carbon chain¹. The hydrophobicity of AHLs relates to their passive diffusion through membranes. AHL-degrading enzymes shows substrate preferences^{2,3}, preferring long chain over short ones, or exhibiting broader specificity spectrum^{4,5}. Moreover, due to several bacteria having more than one QS circuit, the disruption of one QS system does not systematically result in an inhibition of the virulence factor expression, or in the biofilm formation⁶. In fact, regulatory circuits may be interconnected, like in P. aeruginosa⁶, and allow for compensatory responses: if the LasI/LasR system is inactivated, the RhlI/RhlR system can still control LasI/LasR-spectific functions⁷.

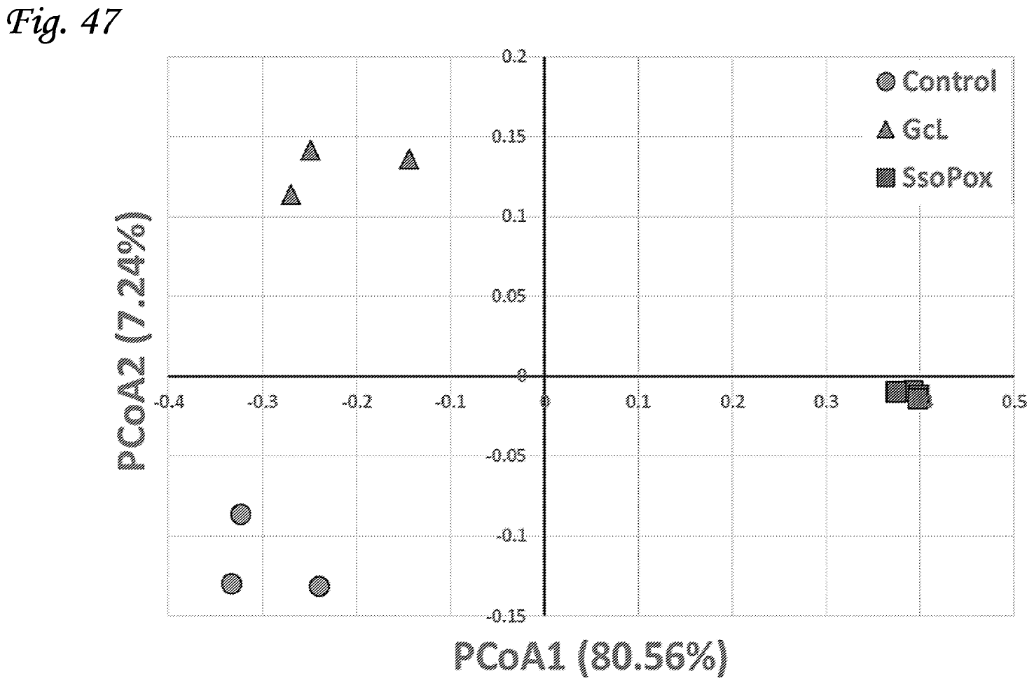

We show that different lactonases change differentially the composition of a complex microbial communities, and that these changes relate not only to the biofilm community, but also to the planktonic bacteria (FIG. 47).

### CITATIONS FOR EXAMPLE 8

1. Lade, H., Paul, D. & Kweon, J. H. N-Acyl Homoserine Lactone-Mediated Quorum Sensing with Special Reference to Use of Quorum Quenching Bacteria in Membrane Biofouling Control. Bio Med Res. Int. 2014, 25 (2014).
2. Afriat, L., Roodveldt, C., Manco, G. & Tawfik, D. S. The latent promiscuity of newly identified microbial lactonases is linked to a recently diverged phosphotriesterase. Biochemistry (Mosc.) 45, 13677-86 (2006).
3. Chow, J. Y. et al. Directed evolution of a thermostable quorum-quenching lactonase from the amidohydrolase superfamily. J Biol Chem 285, 40911-20 (2010).
4. Bergonzi, C., Schwab, M., Chabriere, E. & Elias, M. The quorum-quenching lactonase from Alicyclobacter acidoterrestris: purification, kinetic characterization, crystallization and crystallographic analysis. Acta Crystallogr. Sect. F Struct. Biol. Commun. 73, (2017).
5. Bergonzi, C., Schwab, M. & Elias, M. The quorum-quenching lactonase from Geobacillus caldoxylosilyticus: purification, characterization, crystallization and crystallographic analysis. Acta Crystallogr F Struct Biol Commun 72, 681-6 (2016).
6. Tay, S. B. & Yew, W. S. Development of quorum-based anti-virulence therapeutics targeting Gram-negative bacterial pathogens. Int. J Mol. Sci. 14, 16570-16599 (2013).
7. Dekimpe, V. & Déziel, E. Revisiting the quorum-sensing hierarchy in Pseudomonas aeruginosa: the transcriptional regulator RhlR regulates LasR-specific factors. Microbiology 155, 712-723 (2009).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A metallo-β-lactamase-like lactonase (MLL) comprising at least one amino acid substitution mutation at one or more positions functionally equivalent to M21, W25, Q41, F47, S66, S81, T82, M85, F86, T91, R111, L120, F141, A144, C147, E154, A156, G155, V175, H178, 1182, L183, Y222, I237, M244, or N245 in a reference amino acid sequence SEQ ID NO:1.

2. A protein comprising an amino acid sequence that is at least 80% identical to a reference amino acid sequence SEQ ID NO:1, wherein the protein comprises a lactonase activity, and wherein the protein comprises at least one amino acid substitution mutation at one or more positions functionally equivalent to M21, W25, Q41, F47, S66, S81, T82, M85, F86, T91, R111, L120, F141, A144, C147, E154, A156, G155, V175, H178, 1182, L183, Y222, I237, M244, or N245 in the reference amino acid sequence.

3. The protein of any one of claims 1-2 wherein the protein is a fusion protein.

4. The protein of claim 3 wherein the fusion protein comprises an affinity purification moiety.

5. A polynucleotide comprising: (a) a nucleotide sequence encoding the protein of any one of claims 1-2, or (b) the full complement of the nucleotide sequence of (a).

6. The polynucleotide of claim 5 wherein the polynucleotide is operably linked to at least one regulatory sequence.

7. The polynucleotide of claim 5 wherein the polynucleotide further comprises heterologous nucleotides.

8. A vector comprising the polynucleotide of any one of claims 5-7.

9. A genetically modified microbe comprising an exogenous polynucleotide, wherein the exogenous polynucleotide is a polynucleotide of claims 5-7.

10. The genetically modified microbe of claim 9 wherein the microbe is a E. coli.

11. A composition comprising one or more of the protein of any one of claims 1-2.

12. The composition of claim 11 further comprising a pharmaceutically acceptable carrier.

13. The composition of claim 12 wherein the composition is formulated for parenteral administration or topical administration to an animal.

14. The composition of claim 11 wherein the composition is formulated for foliar administration to a plant.

15. The composition of claim 11 formulated for use as a coating, a cleaning solution, a feed supplement, or a dietary supplement.

16. An article comprising the composition of claim 11.

17. The article of claim 16 wherein the article comprises the composition on a surface of the article.

18. The article of claim 16 wherein the article comprises the composition incorporated into a surface of the article.

19. A method for treating an infection comprising:
administering to an animal having or at risk of having an infection an effective amount of the composition of claim 11.

20. The method of claim 19 wherein the animal is a human.

21. The method of claim 19 wherein the infection is caused by a gram-negative bacterium or a gram-positive bacterium.

22. A method for treating a sign of a condition comprising:
administering to an animal having or at risk of having a condition an effective amount of the composition of claim 11.

23. The method of claim 22 wherein the animal is a human.

24. The method of claim 22 wherein the condition is caused by a gram-negative bacterium or a gram-positive bacterium.

25. A method for treating an infection comprising:
administering to a plant having or at risk of having a bacterial infection an effective amount of the composition of claim 11.

26. The method of claim 25 wherein the plant is a monocot.

27. The method of claim 25 wherein the plant is a dicot.

28. The method of claim 25 wherein the infection is caused by a gram-negative bacterium or a gram-positive bacterium.

29. The method of claim 25 wherein the administering comprises foliar administration.

30. A method for treating a biofilm, comprising:
treating a biofilm present on a surface with an effective amount of one or more proteins of any one of claims 1-2.

31. The method of claim 30 wherein the surface comprises plastic, metal, glass, or a combination thereof.

32. The method of claim 30 wherein the surface is impregnated with the protein.

33. The method of claim 30 wherein at least a portion of the surface is coated with the protein.

34. The method of claim 30 wherein the surface comprises a medical device surface.

35. The method of claim 34 wherein the medical device comprises an endoscope.

36. A method for treating a biofilm, comprising:
treating a surface that is at risk of biofilm formation with an effective amount of one or more proteins of any one of claims 1-2.

37. The method of claim 36 wherein the surface comprises plastic, metal, glass, or a combination thereof.

38. The method of claim 36 wherein the surface is impregnated with the protein.

39. The method of claim 36 wherein at least a portion of the surface is coated with the protein.

40. The method of claim 36 wherein the surface comprises the surface of a medical device.

41. The method of claim 40 wherein the medical device comprises an endoscope.

42. A method for changing the population of a biofilm, comprising:
treating a biofilm with an effective amount of one or more proteins of any one of claims 1-2, or a combination thereof.

43. The method of claim 42 wherein the population is present in a microbiome.

44. A method for reducing spoilage, comprising administering to a fruit, fresh produce, fish, meat, or a dairy product with an effective amount of one or more proteins of any one of claims 1-2.

45. A protein comprising an amino acid sequence that is at least 80% identical to a reference amino acid sequence SEQ ID NO:5, wherein the protein comprises a lactonase activity, and wherein the protein comprises at least one amino acid substitution mutation at one or more positions functionally equivalent to R2, S10, S13, K14, D15, 116, R55, Q58, F59, L90, V91, G93, 1100, L107, L130, 1138, N160, T186, or R241 in the reference amino acid sequence.


SEQUENCE LISTING (15 sequences)

SEQ ID NO: 1 - protein, 250 aa, Parageobacillus caldoxylosilyticus
MTVKKLYFIPAGRCMLDHSSVNSALTPGKLLNLPVWCYLLETEEGPILVDTGMPESAVNNEGLFNGTFVEGQILPKMTEEDRIVNILKRVGYEPDDLLYIISSHLHFDHAGGNGAFTNTPIIVQRTEYEAALHREEYMKECILPHLNYKIIEGDYEVVPGVQLLYTPGHSPGHQSLFIETEQSGSVLLTIDASYTKENFEDEVPFAGFDPELALSSIKRLKEVVKKEKPIIFFGHDIEQEKSCRVFPEYI

SEQ ID NO: 2 - protein, 276 aa, Alicyclobacter acidoterrestris
MGNKLFVLDLGEIRVDENFIIANSTFVTPQKPTVSSRLIDIPVSAYLIQCTDATVLYDTGCHPECMGTNGRWPAQSQLNAPYIGASECNLPERLRQLGLSPDDISTVVLSHLHNDHAGCVEYFGKSRLIAHEDEFATAVRYFATGDHSSPYIVKDIEAWLATPRNWDLVGRDERERELAPGVNLLNFGTGHASGMLGLAVRLEKQPGFLLVSDACYTATNYGPPARRAGVLHDTIGYDRTVSHIRQYAESRSLTVLFGHDREQFASLIKSTDGFYE

SEQ ID NO: 3 - protein, 288 aa, Bacillus thuringiensis
MANVIKARPKLYVMDNGRMRMDKNWMIAMHNPATIHNPNAQTEFVEFPIYTVLIDHPEGKILFDTSCNPNSMGPQGRWAESTQQMFPWTATEECYLHNRLEQLKVRPEDIRYVVASHLHLDHAGCLEMFTNATIIVHEDEFNGALQCYARNQKEGAYIWADIDAWIKNNLQWRTVKRHEDNILLAEGVKVLNFGSGHAWGMLGLHVELPETGGIILASDAIYTAESYGPPIKPPGIIYDSLGYMNTVERIRRIAQETKSQVWFGHDAEQFKKFRKSTEGYYETAGTAG

SEQ ID NO: 4 - protein, 282 aa, Agrobacterium tumefaciens
MTNIAKAQPKLYVMDNGRMRMDKNWMIAMHNPATIANPNAPTEFIEFPIYTVLIDHPEGKILFDTSCNPDSMGAQGRWGEATQSMFPWTASEECYLHNRLEQLKVRPEDIKFVIASHLHLDHAGCLEMFTNATIIVHEDEFSGALQTYARNQTEGAYIWGDIDAWIKNNLNWRTIKRDEDNIVLAEGIKILNFGSGHAWGMLGLHVQLPEKGGIILASDAVYSAESYGPPIKPPGIIYDSLGFVRSVEKIKRIAKETNSEVWFGHDSEQFKRFRKSTEGYYE

SEQ ID NO: 5 - protein, 316 aa, Sulfolobus solfataricus
GMVRISIAGGNEIDPGSMGLTLFHEHLRLITEVVRWNWPHLYNEDEELKRAIDAVNAAKKYGVKTIIDLTVAGIGCDVRFNEKVAKATGVNIIMGTGFYTYTEIPFYFKNRGIDSLVDAFVHDITIGIQGTNTRAAFVKAVIDSSGLTKDVEMAIRAAAKAHIKTDVPIITHSFVGNKSSLDLIRIFKEEGVDLARTVIGHVGDTDDISFIEQILREGAFIGLDRFGLDIYLPLDKRVKTAIELIKRGWIDQLLLSHDYCPTIDWYPPEVVRSTVPDWTMTLIFEKVIPRMRSEGITEEQINRVLIDNPRRLFTGR

SEQ ID NO: 6 - protein, 314 aa, Sulfolobus islandicus
MRIPLVGKDSIESKDIGFTLIHEHLRVFSEAVRQQWPHLYNEDEEFRNAVNEVKRAMQFGVKTIVDPTVMGLGRDIRFMEKVVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLAPRWSITLIFEDTIPFLKRNGVNEEVIATIFKENPKKFFS

SEQ ID NO: 7 - protein, 315 aa, Vulcanisaeta moutnovskia
MTKIPLVGKGEISPGEMGFTLIHEHLRVFSEPVRYQWPHLYNEDEELKNAVNEVKTIMSYGVKTIVDPTVMGLGRDIRFSEKVVKETGINVIAATGLYTYTDLPFFFNGRSLEEIAELLIHDIKKGIQGTNNRAGFIKVAADEPGITRDVERAIRAAAIAQKETNVPIITHSNAHNGTGLEQQRILMEEGVDPGRVLIGHLGDTDNVDYIKKIADKGSFVGLDRYGLDLFLPIDKRNEVLLKLIKDGYLDRIMVSQDYCCTIDWGIAKPEYKPKLAPKWSMSLIFTDVIPSIKRAGVTDEQLHVIFVKNPARLFS

SEQ ID NO: 8 - protein, 8 aa, Artificial
  strep tag
WSHPQFEK

SEQ ID NO: 9 - protein, 7 aa, Artificial
  TEV sequence
ENLYFQS

SEQ ID NO: 10 - protein, 282 aa, Artificial
  GcL ancestor
MTNIVKARPKLYVMDNGRMRMDKNWMIAMHNPATIHNPNAPTEFVEFPIYTVLIDHPEGKILFDTACNPNSMGPQGRWAEATQQMFPWTASEECYLHNRLEQLKVRPEDIKFVVASHLHLDHAGCLEMFTNATIIVHEDELNGTLQCYARNQKEGAYIWADIDAWIKNNLQWRTIKRHEDNLLLAEGIRVLNFGSGHAWGMLGLHVELPETGGIILASDAIYTAESYGPPIKPPGIIYDSLGYVNTVEKIRRIAKETNSQVWFGHDAEQFKQFRKSTEGYYE

SEQ ID NO: 11 - protein, 314 aa, Artificial; identical to SEQ ID NO: 6
  SsoPox ancestor

SEQ ID NO: 12 - protein, 314 aa, Artificial
  SsoPox6 variant
MKIPLVGKDEIEPRDIGFTLIHEHLRVFSEAVRQQWPHLYNEDEEFRNAVNEVKRAMQFGVKTIVDPTVMGLGRDIRFMEKVVKATGINLIAGTGIYIYIDLPFYFNNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLAPRWSITLIFEDTIPFLKRNGVNEEVIATIFKENPKKFFS

SEQ ID NO: 13 - protein, 314 aa, Artificial
  SsoPox19 variant
MKIPLVGKDEIEPREMGFTLIHEHLRVFSEAVRQQWPHLYNEDEEFRNAVNEVKTAMSYGVKTIVDPTVMGLGRDIRFMEKVVKATGINVIAATGIYIYTDLPFYFNNRSIDEIADLFIHDIKEGIQGTNNKAGFVKVAADEPGITKDVEKVIRAAAIAHKETKVPIITHSNAHNNTGLEQQRILMEEGVDPGKILIGHLGDTDNIDYIKKIADKGSFIGLDRYGLDLFLPVDKRNETTLKLIKDGYSDKIMISHDYCCTIDWGTAKPEYKPKLAPRWSITLIFEDTIPFLKRNGVNEEVIATIFKENPKKFFS

SEQ ID NO: 14 - protein, 19 aa, Artificial
  primer
GTGCCAGCMGCCGCGGTAA

SEQ ID NO: 15 - protein, 6 aa, Artificial
  dinuclear metal binding motif
  X (2)..(4): Any amino acid
HXHXDH